(19) United States
(12) Patent Application Publication
(10) Pub. No.: US 2025/0191691 A1
(43) Pub. Date: Jun. 12, 2025

(54) METHODS FOR THE TRANSLATED ALIGNMENT OF TRANSCRIPTOMIC DATA AT SINGLE-CELL RESOLUTION

(71) Applicant: California Institute of Technology, Pasadena, CA (US)

(72) Inventors: Laura Luebbert, Pasadena, CA (US); Lior S. Pachter, Pasadena, CA (US); Delaney Sullivan, Pasadena, CA (US)

(21) Appl. No.: 18/972,306

(22) Filed: Dec. 6, 2024

Related U.S. Application Data
(60) Provisional application No. 63/607,237, filed on Dec. 7, 2023.

(51) Int. Cl. G16B 30/10; C12Q 1/70; G16B 40/30

(52) U.S. Cl. CPC G16B 30/10; C12Q 1/70; G16B 40/30; C12Q 2600/112

(57) ABSTRACT

Disclosed herein include method and systems suitable for use in detection and/or prediction of microorganisms in a sample using sequencing data. In some embodiments, the method comprises converting reference sequences and sample sequences to comma-free sample codes; and detecting the presence of microbes in the sample based on alignment of the comma-free codes. In some embodiments, the method comprises detecting and/or predicting viral presence in host cell, by identifying and analyzing signature host genes. In some embodiments, the system suitable for performing the methods disclosed herein.

### RELATED APPLICATIONS

This application claims the benefit under 35 U.S.C. § 119(e) of U.S. Provisional Patent Application Ser. No. 63/607,237, filed Dec. 7, 2023, the content of this related application is incorporated herein by reference in its entirety for all purposes.

### STATEMENT REGARDING FEDERALLY SPONSORED R&D

This invention was made with government support under contract No. T32 GM008042 and F30AI167524 awarded by National Institutes of Health (NIH), and under grant No. 2139433 awarded by National Science Foundation (NSF). The government has certain rights in the invention.

### BACKGROUND

### Field

The present disclosure relates generally to the field of genomic and/or proteomic analysis including systems and methods for detecting microorganisms in a sample using sequencing data.

### Description of the Related Art

More than 300,000 mammalian virus species are estimated to cause disease in humans. They inhabit human tissues such as the lungs, blood, and brain and often remain undetected. Efficient and accurate detection of viral infection is vital to understanding its impact on human health and to make accurate predictions to limit adverse effects, such as future epidemics. The increasing use of high-throughput sequencing methods in research, agriculture, and healthcare provides an opportunity for the cost-effective surveillance of viral diversity and investigation of virus-disease correlation. However, existing methods for identifying viruses in sequencing data rely on and are limited to reference genomes or cannot retain single-cell resolution. Therefore, there is a need for improved methods for detecting novel microbes (e.g., viruses), while retaining single-cell resolution.

### SUMMARY

Disclosed herein include methods for detecting microbes in a sample. In some embodiments, the method comprises: converting a plurality of reference sequences to a plurality of comma-free reference codes; converting a plurality of sample sequences to a plurality of comma-free sample codes; and aligning the plurality of comma-free reference codes to the plurality of comma-free sample codes to generate a microbe profile of the sample, thereby detecting the presence of one or more microbes in the sample.

The method can further comprise removing sample sequences of the plurality of sample sequences originated from host. In some embodiments, removing sample sequences of the plurality of sample sequences originated from host comprises removing sample sequences of the plurality of sample sequences aligned to host sequences to obtain a plurality of pre-aligned sample sequences. In some embodiments, converting the plurality of sample sequences to the plurality of comma-free sample codes comprises converting the plurality of pre-aligned sample sequences to the plurality of comma-free sample codes. The method can further comprise: converting host sequences to a plurality of comma-free host codes; and aligning the plurality of comma-free sample codes to the comma-free host codes. In some embodiments, converting the host sequences to the plurality of comma-free host codes comprises converting each reading frame of the host sequences to comma-free codes. In some embodiments, the host sequences comprise genome sequence, transcriptome sequence or a combination thereof. In some embodiments, the plurality of comma-free host codes comprise a shared sequence with the plurality of comma-free reference codes and a host specific sequence. The method can further comprise removing comma-free sample codes of the plurality of comma-free sample codes that comprise a portion aligned to the host specific sequence. The method can further comprise removing comma-free sample codes of the plurality of comma-free sample codes that lack a reference specific sequence, wherein the reference specific sequence aligns to the plurality of comma-free reference codes but not the comma-free reference codes comma-free host codes.

In some embodiments, aligning the plurality of comma-free reference codes to the plurality of comma-free sample codes comprises determining similarity between the plurality of comma-free reference codes and the plurality of comma-free sample codes. In some embodiments, aligning the plurality of comma-free reference codes to the plurality of comma-free sample codes comprises selecting the comma-free sample codes of the plurality of comma-free sample codes having at least 50% similarity to the comma-free reference codes of the plurality of comma-free reference codes for subsequent analysis.

The method can further comprise comparing the alignment of the plurality of comma-free sample codes associated with a sample sequence of the plurality of sample sequences to the plurality of comma-free reference codes. The method can further comprise selecting the comma-free sample codes of the plurality of comma-free sample codes having the highest similarity to the comma-free reference codes compared to other comma-free sample codes associated with the same sample sequence for subsequence analysis.

In some embodiments, the sample comprises cells that are infected or suspected to be infected with microbes. In some embodiments, the plurality of reference sequences comprise amino acid sequences and/or nucleic acid sequences. In some embodiments, the plurality of reference sequences comprise amino acid sequences conservative in virus. In some embodiments, the plurality of reference sequences comprise RNA-dependent RNA polymerase (RdRp)-containing amino acid sequences and/or antimicrobial amino acid sequences. In some embodiments, the length of each of the plurality of comma-free reference codes is 10-3000 nucleotides. In some embodiments, the length of each of the plurality of comma-free reference codes is 31 nucleotides. In some embodiments, the plurality of reference sequences are clustered into species-like operational taxonomic units (sOTUs). In some embodiments, the sOTUs comprises taxonomy source of each of the plurality of references sequences. The method can further comprise removing duplicate comma-free reference codes of the plurality of comma-free reference codes. In some embodiments, the plurality of reference sequences comprise sequences from at least 9,000 species. In some embodiments, each of the plurality of comma-free reference sequences comprises taxonomy source information of its corresponding reference sequence.

In some embodiments, the plurality of sample sequences comprise amino acid sequences and/or nucleic acid sequences. In some embodiments, the plurality of sample sequences comprise mRNA sequences obtained from a single cell. In some embodiments, each of the plurality of sample sequences comprises a cell barcode and/or a unique molecular identifier (UMI). In some embodiments, the cell barcodes associated with the same cell are the same, and wherein the cell barcodes associated with different cells are different. In some embodiments, the UMIs associated with the same cell are different. In some embodiments, the plurality of sample sequences comprise at least one mutation. In some embodiments, the mutation is an insertion, a deletion and/or a substitution of at least one nucleotide or an amino acid. In some embodiments, the mutation is a point mutation and/or a silent mutation. In some embodiments, the mutation rate of the plurality of sample sequences is no greater than 20%. In some embodiments, the mutation rate of the plurality of sample sequences is no greater than 12%.

In some embodiments, converting the plurality of reference sequences to the plurality of comma-free reference codes comprises converting each reading frame to a comma-free code, and/or wherein converting the plurality of sample sequences to the plurality of comma-free sample codes comprises converting each reading frame to a comma-free code.

In some embodiments, the microbe profile comprises taxonomy of the microbes. In some embodiments, generating the microbe profile comprises assigning the microbe to a species-like operational taxonomic units (sOTUs). The microbe profile can comprise the number of microbes, the number of microbes in each sOTUs, and/or the tropism of the microbes.

The method can further comprise determining profile of the cells. In some embodiments, the profile of the cells comprises transcriptome profile. In some embodiments, the profile of the cells comprises expression level of genes known to be associated with microbe infection. In some embodiments, the genes known to be associated with microbe infection are MS4A1, CD19, CD79B, MZB1, IRF8, CD1C, IL7R, CD8A, CD3D, CD3G, CD3E, CD4, GZMB, KLRB1, NCR1, FCGR3, HLA-DRB5, HLA-DRA, CD68, ITGAX, CD14, ITGAM, CFD, CD163, SOD2, LCN2, CD4177, CD45, IL-10, CCL2, CCL3, CCL4 and/or Ki67. The method can comprise determining the percentage of cells infected with the microbe. The profile of the cells can comprise type of cells infected with the microbe and abundance of each type of cells infected with the microbe. The method can comprise determining the stage of microbe infection.

In some embodiments, the method detects more microbes compared to a method aligning the plurality of sample sequences to NCBI reference sequences. The method can, e.g., detect microbes without a sequence included in the NCBI database. In some embodiments, the method detects microbes without a sequence included in the plurality of reference sequences. In some embodiments, the method generates microbe profile with at least 90% accuracy.

Disclosed herein include methods for predicting or detecting microbes in a sample. The method can comprise: providing a model with a training dataset to determine a weight of each gene in the training data, wherein the model is a logistic regression modal, and wherein the training dataset comprises sequencing data of one or more cells; determining one or more signature genes, wherein the signature genes have weights no less than a threshold; providing a trained model with a testing dataset, wherein the trained model is parameterized with the weight of the signature genes and wherein the testing dataset comprises sequencing data of one or more cells in the sample; and determining a probability of presence of the microbes using the trained model, thereby determining the presence or absence of the microbes in the sample.

In some embodiments, the sample comprises one or more cells that is infected or suspected to be infected with microbes. In some embodiments, the microbe is a virus. In some embodiments, the virus is a virus from the realm of Riboviria. In some embodiments, the virus is selected from the group consisting of Duplornaviricota, Kitrinoviricota, Lenarviricota, Negamaviricota, Peploviricota and Fusariviridae. In some embodiments, the virus is selected from coronaviruses, dengue viruses, ebolaviruses, hepatitis B viruses, influenza viruses, measles viruses, mumps viruses, polioviruses, West Nile viruses and Zika viruses.

In some embodiments, the sequencing data comprises sequencing data of transcriptome of the one or more cells. In some embodiments, the training dataset comprises cell type of each cell of the one or more cells. In some embodiments, the training dataset comprises infection status of each cell of the one or more cells. In some embodiments, infection status comprises the presence or absence of microbes, taxonomy of the microbes, and stage of infection. In some embodiments, the training dataset comprises all genes in the one or more cells. In some embodiments, the training dataset comprises highly variable genes in the one or more cells.

In some embodiments, the testing dataset comprises sequencing data of transcriptome of the one or more cells in the sample. In some embodiments, the testing dataset comprises cell type of each cell of the one or more cells in the sample.

In some embodiments, the threshold is 0.01. In some embodiments, the threshold is 0.05. In some embodiments, the threshold is 0.2. In some embodiments, the signature genes are genes encoding: proteins regulating cytokine production, proteins regulating viral entry into host cell, proteins regulating viral life cycle, and/or receptors mediating endocytosis. In some embodiments, the signature genes are genes encoding proteins selected from FCN1, GSN, EML1, ARFGEF2, CD14, SLAMFI, FCRL3, UBASH3A, RGCC, LMNA, NCAPG, FCRL3, DAND5, CTSL, MAPK11, VCL, TOGARAM1 and KIF18A.

In some embodiments, accuracy of determining the presence or absence of microbes in the sample is at least 60%. In some embodiments, determining the presence or absence of microbes in the sample comprises determining whether the presence or absence of microbes in each of the one or more cells in the sample. In some embodiments, determining the presence or absence of microbes in the sample comprises determining taxonomy of the microbes. In some embodiments, determining the presence or absence of microbes in the sample comprises determining the number of microbes. In some embodiments, determining the presence or absence of microbes in the sample comprises determining the number of each microbe species in each cell of the one or more cells in the sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts non-limiting exemplary embodiments and data related to a schematic overview of the kallisto translated search front- and back-end. The front-end was similar to kallisto-bustools workflows. The user provided sequencing data (e.g., FASTQ files), as well as a reference data (e.g., FASTA file) containing amino acid sequences to align the sequencing data (e.g., nucleic acid sequencing data) against. In the kallisto translated search back-end, the reference amino acid sequences (e.g., PalmDB) and the nucleic acid sequencing reads (e.g., user-generated data) were translated into non-redundant comma-free codes. For the nucleic acid sequences, the translation occurred in all six possible reading frames, including three forward and three reverse frames. The pseudoalignment was performed in the comma-free code space and was compatible with the kallisto cell barcode tracking, which enabled analysis at single-cell resolution. The workflow generated a cell barcode by virus ID count matrix.

FIG. 2A-FIG. 2B depict non-limiting exemplary embodiments and data related to a comparison of the method and system disclosed herein and other available database and/or tools. FIG. 2A depicts a phylogenetic tree of the taxonomies of viral sequences/genomes included in the PalmDB species-like operational taxonomic units (sOTUs) and NCBI RefSeq databases from phylum to genus. Barplots indicate the number of sequences/species available for each taxonomy in each database. The tree was generated with iTOL. This plot can also be viewed interactively at tinyurl.com/. The taxonomies and numbers of viral sequences/genomes in FIG. 2A is included in Table 5. The bar graph illustrates the numbers of viral sequences/genomes, with the inner part of each bars showing number of sequences in NCBI RefSeq and the outer part of each bars showing number of sequences in PalmDB. FIG. 2B depicts a comparison of the performance of kallisto (e.g., translated search and standard workflow) and Kraken2 at different mutation rate. Mutation-Simulator was used to introduce random single nucleotide base substitutions to 676 ZEBOV RdRP sequences obtained by Seq-Well sequencing at increasing mutation rates. 10 simulations per mutation rate were performed. The sequences were subsequently aligned using kallisto translated search against the complete PalmDB, Kraken2 translated search against the RdRP amino acid sequence of ZEBOV with a manually adjusted NCBI Taxonomy ID to allow compatibility with Kraken2, and kallisto standard workflow against the complete ZEBOV nucleotide genome (GCA_000848505.1). The plot shows the recall percentage of the 676 sequences for each of the 10 simulations at each mutation rate. Each was fitted with an inverse sigmoid for mutation rates >0.

FIG. 3A-FIG. 3B depict non-limiting exemplary embodiments and data related to the compatibility of the method and system disclosed herein with various sequencing technologies. FIG. 3A depicts a comparison of performance of kallisto on data obtained with different sequencing technologies. Sequencing data from samples with a known viral infection obtained using different bulk and single-cell RNA sequencing technologies was aligned to PalmDB using kallisto translated search. Viral loads obtained through alternative methods, such as RNA-ISH and qPCR, were compared to the target virus counts returned by kallisto. The top left panel shows RNA-ISH (%) over total raw kallisto counts for SARS-CoV for 23 lung autopsy samples from COVID-19 patients obtained by bulk RNA sequencing. Error bars show min-max values for each read in a pair; the dot shows the mean. The top right panel shows SARS-CoV-2 viral load by RT-qPCR (copies/mL) over total raw kallisto counts for SARS-CoV species obtained by bulk RNA sequencing of 10 saliva (circle), 3 nasal swab (triangle) and 3 throat swab (star) specimens from patients with acute SARS-CoV-2 infection. Each specimen underwent duplicate library preparation and paired-end sequencing; points (e.g., circle, triangle and star shapes) indicate the mean among the paired reads and duplicates, and error bars show min-max values. The bottom left panel shows total raw kallisto counts for SARS-CoV species of 3 human iPSC-derived cardiomyocytes infected with SARS-CoV-2 and 3 control samples obtained by SMART-Seq. The bottom right panel shows RT-qPCR (copies/mL) over total raw kallisto counts for ZEBOV in 19 rhesus macaque blood samples obtained at different stages of infection with ZEBOV and sequenced with Seq-Well. FIG. 3B depicts the robustness of taxonomic assignment. To validate the mapping of nucleotide sequences to an amino acid reference with kallisto translated search and assess the robustness/accuracy of the taxonomic assignment, all amino acid sequences in the PalmDB were reverse translated using the “standard” genetic code. The reverse translated PalmDB RdRP sequences were subsequently aligned to the optimized PalmDB amino acid reference with kallisto translated search. For each sequence, the mapping result was differentiated at each taxonomic rank into four categories: “correct” or “incorrect” taxonomic assignment based on the sOTU to virus ID mapping; “multimapped,” which refers to the alignment of a sequence to multiple targets in the reference and incapability to unambiguously assigned the sequence to one target; or “not aligned,” which refers to that the sequence was not aligned. The plot shows the fraction of sequences falling into each mapping result category assessed at each taxonomic rank. The numbers of sequences above the bars indicate the total number of sequences per rank, which is also summarized in Table 6. Family names and numbers were omitted, and genera and species ranks were combined for readability.

FIG. 4A-FIG. 4C depict non-limiting exemplary embodiments and data related to host masking options. FIG. 4A depicts a schematic overview of the different host masking options disclosed herein. Reads that align to PalmDB and are considered viral are marked with a ** and reads that align to the host genome or transcriptome are marked in black or grey bars without **, respectively. * in FIG. 4A indicates that the hosting masking method also captured instances where the viral and host fractions of a read were not flanking. The barplot shows the number of distinct sOTUs, defined by distinct virus IDs observed in ≥0.05% of cells for each workflow. FIG. 4B depicts a schematic overview of masking the host genome with the D-list argument when used in combination with translated search. The D-list genome consisted of nucleotide sequences and hence was translated to comma-free code in all six possible reading frames, similar to the translation of the nucleic acid sequencing reads. FIG. 4C depicts the generation of two distinct virus count matrices by masking host sequences with the kallisto read capture workflow. The first virus count contained viral reads that only aligned to the PalmDB, and the second contained viral reads that aligned to the host transcriptome in addition to the PalmDB. The majority of viruses detected above the quality control (QC) threshold (observed in ≥0.05% of cells), had reads that aligned to the host transcriptome as well as the PalmDB. The barplot shows the fraction of reads for each virus that aligned to the PalmDB only (“virus only,” the bottom part of each bar) and those that aligned to the host transcriptome in addition to the PalmDB (“also in host,” the top part of each bar). In the right panel of FIG. 4C, area A includes 8,916 viral reads not also aligned to host; area B includes 3,006 viral reads that also wholly aligned to host; and area C includes 2,260 viral reads partially aligned to host. 96.5% of viruses expressed above the QC threshold fell into area C, as shown by the arrow pointing to area C. From left to right, the virus ID in the left panel of FIG. 4C include: u10 (ZEBOV), u288819, u10240, u135858, u11150, u101227, u202260, u102540, u181379, u290519, u100599, u110641, u1001, u39566, u100145, u100076, u100007, u100173, u100074, u100093, u100251, u100291, u27694, u100116, u100026, u100302, u134800, u102324, u100001, u100289, u100245, u100024, u100733, u100177, u100644, u100154, u100031, u100048, u100296, u100011, u100012, u10015, u100019, u100188, u100153, u100002, u100028, u100111, u103829, u100017, u100000, u100004, u100049, u100139, u100196, u34159 and u183255.

FIG. 5A-FIG. 5B depict non-limiting exemplary embodiments and data related to the performance of host masking workflows. FIG. 5A depicts the number of positive cells obtained for 12 different virus IDs by each masking workflow. The cell counts for all viruses detected above the QC threshold for all masking workflows are shown in FIG. 13. FIG. 5B depicts pyCirclize plots showing the BLAST+ results of randomly selected sequencing reads for each of the novel viruses shown in FIG. 5A, except the known virus ZEBOV (virus ID “u10”). Each circular plot corresponds to the results for one virus ID. Each light grey sector corresponds to one sequencing read that linked to the super-kingdoms including eukaryotes (the color of Monkey in the first circular plot), bacteria (the color of bacterial cell in the first circular plot), viruses (the color of virus in the first circular plot) and archaea sectors, based on its BLAST+ alignment results. The width of the connecting link indicates the BLAST+ alignment coverage percentage, and its color indicates the identity percentage. For u202260, approximately two third of the extracted reads yielded no BLAST results.

FIG. 6A-FIG. 6D depict non-limiting exemplary embodiments and data related to the identification of viral infected host cells. FIG. 6A depicts a schematic overview of the single-cell RNA sequencing data collected by Kotiar et al. Kotliar et al. performed single-cell RNA sequencing of peripheral blood mononuclear cell (PBMC) samples from 19 rhesus macaques at different time points during Ebola virus disease (EVD) after infection with Zaire Ebolavirus (ZEBOV) using Seq-Well with the S3 protocol. A subset of the PBMC samples were spiked with Madin-Darby canine kidney (MDCK) cells. This schematic was adapted from the original design by Kodiar et al. FIG. 6B depicts a plot of the percentage of positive MDCK cells for each virus against the percentage of positive macaque cells. The viruses were categorized into “shared,” “macaque only,” “MDCK only,” and “undefined” viruses. The right panel shows the same plot without log scale axes such that zero counts are included. FIG. 6C depicts a bar plot showing the fraction of positive cells obtained for each virus order, as defined by the PalmDB sOTUs, for each virus category. The virus order in FIG. 6C are: 1. Articulavirales; 2. Cryppavirales; 3. Durnavirales; 4. Ghabrivirales; 5. Herpesvirales; 6. Levivirales; 7. Martellivirales; 8. Ourlivirales; 9. Reovirales; 10. Sobelivirales; 11. Tolivirales; 12. Picomavirales; 13. Wolframvirales; 14. Amarillovirales; 15. Bunyavirales; 16. Undefined; 17. Nidovirales; and 18. Mononegavirales. FIG. 6D depicts fraction of positive cells for all “macaque only” and “shared” viruses. Each row corresponds to one animal at a specific EVD time point. The fractions were scaled to range from zero to one for each virus. The raw total fraction of positive cells for each virus across all samples is shown as the bottom row.

FIG. 7A-FIG. 7C depict non-limiting exemplary embodiments and data related to a profile of viral infected cells. FIG. 7A depicts the fraction of cells occupied by each EVD time point per Leiden cluster. Each Leiden cluster was assigned a cell type based on previously defined marker genes as shown in FIG. 11D. On the right, the number of distinct viruses, defined by different sOTUs/virus IDs, detected in each cell is shown. Each grey dot in the right panel of FIG. 7A corresponds to one cell, and the black dot corresponds to the mean across all cells. FIG. 7B depicts the number of ZEBOV (u10) positive cells per 10,000 cells per EVD time point (top panel) and per cell type (third panel from top). The number of cells are indicated next to each bar in the top and third from top panels. For each time point and cell type, the number of distinct viruses, defined by different sOTUs/virus IDs, found per cell is plotted as shown in the second panel from top and the bottom panel. Each grey dot corresponds to one cell, and the black dot corresponds to the mean across all cells. FIG. 7C depicts the number of positive cells per 10,000 cells per cell type for the 6 novel viruses. Virus IDs that show relatively high cell type specificity are shown on the left, and virus IDs with relatively even detection across all cell types are shown on the right.

FIG. 8A-FIG. 8C depict non-limiting exemplary embodiments and data related to the prediction of the presence of virus in host cell. FIG. 8A depicts the abundance of each virus-like sequence from the same animal taken at two time points. Several animals included in the macaque PBMC dataset were sampled twice, at two different time points. Here, for each virus, the percentage of positive cells occupied by the later time point is shown. The number of positive cells for each virus was first normalized to the total number of cells in the sample. Only viruses for which at least one time point had positive counts were included for each animal. A percentage of 50% (dashed line) indicates that the number of positive cells for that virus remained stable between the two time points. FIG. 8B depicts the prediction of viral presence based on host gene expression. Logistic regression models were trained to predict the presence of specific viruses based on host gene expression at single-cell resolution. The accuracy of the logistic regression model trained on highly variable (11V) macaque genes with donor animal and EVD time point as covariates is shown for the known virus ZEBOV (u10) and 6 novel viruses. The presence of viruses that displayed high cell type specificity could be predicted with >70% accuracy, while viruses with low cell type specificity could not be predicted above random chance (50%, marked by the dashed line). As a negative control, viral presence and absence labels were scrambled at random in the training data, causing the prediction accuracy to drop to random chance (50%), as expected. The bottom bar plots show the number of testing and training cells for each virus (also see FIG. 16C). FIG. 8C depicts a heatmap of the prediction accuracy across all possible modeling combinations (e.g., training on all macaque genes versus only highly variable (HV) genes, and with or without covariates donor animal and EVD time point). The prediction accuracy remains stable across all modeling choices.

FIG. 9 depicts non-limiting exemplary embodiments and data related to 676 ZEBOV RdRP sequences identified by aligning a subset of 100,000,000 single-cell RNA sequencing reads of macaque PBMC samples obtained 8 days after infection with ZEBOV to the optimized PalmDB using kallisto translated search. The sequences were subsequently aligned to PalmDB reference indices, from which (i) all Ebolavirus species were removed (darkest color), (ii) all Ebolavirus genera were removed (medium dark color), or (iii) all Filoviridae were removed (lightest color). In each scenario, a subset of sequences aligned to the nearest remaining relative based on the main taxonomic rank, suggesting that kallisto translated search can detect the highly conserved RdRP of a large number of viral species, beyond the number of sequences in the PalmDB database, while still providing reliable sOTU-based taxonomic assignment of lower-rank taxonomies.

FIG. 10 depicts non-limiting exemplary embodiments and data related to a visualization of the identification of RdRP sequences with kallisto translated search. A subset of 100,000,000 reads obtained using Seq-Well sequencing of macaque peripheral blood mononuclear cell (PBMC) samples obtained at 8 days post-infection with ZEBOV was selected. The reads were aligned to the PalmDB amino acid sequences with kallisto translated search. The reads were also aligned to the complete ZEBOV nucleotide genome using a standard nucleotide alignment (e.g., Kraken2). Aligned reads from both alignments were extracted and realigned to the ZEBOV genome using bowtie2, a BAM file was created using SAMtools, and the alignment was subsequently visualized NCBI Genome Workbench.

FIG. 11A-FIG. 11E depict non-limiting exemplary embodiments and data related to host count matrix and marker genes. FIG. 11A depicts knee plot of sorted total UMI counts per cell and library saturation plot of host (e.g., rhesus macaque and MDCK) cells sequenced by Kotliar et al. FIG. 11B depicts Canis lupus (dog/MDCK) over Macaca mulatta (macaque) UMI count for each cell. Cells were categorized as macaque if a maximum of 10% of their UMIs originated from dog genes and vice versa. FIG. 11C depicts the obtained numbers of macaque, dog (MDCK) and uncategorized cells after species separation. FIG. 11D depicts mean expression of marker genes used for cell type assignment per macaque Leiden cluster. The barplot shows the number of cells in each cluster. Cluster “Undefined 1” was omitted because it only contained 12 cells. FIG. 11E depicts frequency of host and viral gene counts in individual cells.

FIG. 12A-FIG. 12C depict non-limiting exemplary embodiments and data related to the effects of different host masking options. FIG. 12A depicts precision of species-level (top) and genus-level (bottom) taxonomic assignment at increasing simulated mutation rates. Mutation-Simulator was used to add random single nucleotide base substitutions to 676 ZEBOV RdRP sequences obtained by Seq-Well sequencing at increasing mutation rates. 10 simulations per mutation rate were performed. The sequences were subsequently aligned using kallisto translated search against the complete PalmDB. FIG. 12B depicts the recall percentages at each mutation rate. Fraction of counts were obtained for the known viral infection (e.g., SARS-CoV-2) and per viral strandedness per primer type. Lung samples from mice infected with SARS-CoV2 were sequenced with SPLiT-Seq and aligned to PalmDB using kallisto translated search using the D-list to mask the host (e.g., mouse) genome. The plot shows the fraction of counts obtained for SARS-CoV as well as all sOTUs of different strandedness per primer type. FIG. 12C depicts the de Bruijn graph generated from the reverse translated PalmDB sequences in the kallisto translated search workflow, visualized and colored using Bandage v0.8.1.

FIG. 13 depicts non-limiting exemplary embodiments and data related to the number of positive cells for each individual virus ID (Table 1) obtained by different host masking options. Each virus ID shown in FIG. 13 was observed in ≥0.05% of cells. The host masking options are visualized in FIG. 4A.

FIG. 14 depicts non-limiting exemplary embodiments and data related to the number of positive cells per 10k cells for viral species of genera known to infect rhesus macaques in the data from Kotliar et al. analyzed using kallisto translated search with PalmDB. Host sequences were masked using the D-list option with the host genomes and transcriptomes, followed by host read capture using kallisto. No quality control thresholding of virus-like sequences was performed prior to generating this plot and the majority of these virus-like sequences were filtered out during quality control, and identified as contaminating sequences. It is notable that Flaviviruses was detected. Since the genomes of Flaviviruses are often not polyadenylated, they should not be captured by polyA capture-dependent single-cell RNA sequencing technologies, such as Seq-Well used herein. It is possible that these RNA molecules were spuriously captured even though they were not polyadenylated. Alternatively, Flaviviruses was captured, since the presence of a polyA-tail has been reported for some Flavivirus strains. The figure legend in FIG. 14 include the following:

In the panel of Orthoreovirus: 1—Piscine orthoreovirus, 2—Piscine orthoreovirus 3, 3—Mammalian orthoreovirus, 4—Avian orthoreovirus, 5—undefined, and 6—Pteropine orthoreovirus.

In the panel of Deltacoronavirus: 1—Sparrow deltacoronavirus, 2—Undefined, 3—Coronavirus HKU15, and 4—Quail coronavirus UAE-HKU30.

In the panel of Arterivirus: 1—Betaarterivirus suid 2, 2—Deltaarterivirus pejah, 3—Etaarterivirus ugarco 1, 4—Epsilonarterivirus safriver, 5—Deltaarterivirus hemfev, 6—Kappaarterivirus wobum, 7—Undefined, 8—Thetaarterivirus mikelba 1, 9—Alphaarterivirus equid, and 10—Gammaarterivirus lacdeh.

In the panel of Rotavirus: 1—Rotavirus I, 2—Rotavirus C, 3—Rotavirus H, 4—Rotavirus F, 5—Murine rotavirus, 6—Rotavirus D, 7—Tasmanian devil-associated rotavirus 1, 8—Rotavirus A, 9—Rotavirus B, 10—Undefined, and 11—Rotavirus G.

In the panel of Gammacoronavirus: 1—Avian coronavirus, 2—Undefined, and 3—Beluga whale coronavirus SW1.

In the panel of Morbillivirus: 1—Measles morbillivirus, 2—Feline morbillivirus, 3—Canine morbillivirus, 4—Rinderpest morbillivirus, 5—Small ruminant morbillivirus, 6—Cetacean morbillivirus, 7—Phocine morbillivirus, 8—Feline morbillivirus type 2, and 9—Undefined.

In the panel of Cardiovirus: 1—Cardiovirus A, 2—Cardiovirus B, 3—Undefined, and 4—Cardiovirus C.

In the panel of Orthohepevirus: 1—Orthohepevirus A, 2—Undefined, 3—Orthohepevirus B, and 4—Orthohepevirus C.

In the panel of Enterovirus: 1—Avian metapneumovirus, 2—Undefined, and 3—Human metapneumovirus.

In the panel of Metapneumovirus: 1—Enterovirus A, 2—Enterovirus C, 3—Enterovirus D, 4—Enterovirus B, 5—Rhinovirus C, 6—Enterovirus J, 7—Enterovirus G, 8—Human rhinovirus sp., 9—Goat enterovirus, 10—Enterovirus E, 11—Enterovirus F, 12—Enterovirus sp., 13—Enterovirus H, 14—Rhinovirus A, 15—Rhinovirus B, and 16—Undefined.

In the panel of Piscihepevirus: 1—Piscihepevirus A.

In the panel of Respirovirus: 1—Human respirovirus 1, 2—Murine respirovirus, 3—Bovine respirovirus 3, 4—Human respirovirus 3, 5—Undefined, and 6—Porcine respirovirus 1.

In the panel of Hepatovirus: 1—Undefined, 2—Hedgehog hepatovirus, and 3—Hepatovirus A.

In the panel of Arenavirus: 1—Mopeia Lassa virus reassortant 29, and 2—Undefined.

In the panel of Alphainfluenzavirus: 1—Influenza A virus, and 2—Undefined.

In the panel of Sapelovirus: 1—Sapelovirus A, 2—Sapelovirus B, and 3—Undefined.

In the panel of Mammarenavirus: 1—Guanarito mammarenavirus, 2—Lujo mammarenavirus, 3—Cali mammarenavirus, 4—Tacaribe mammarenavirus, 5—Pirital mammarenavirus, 6—Lassa mammarenavirus, 7—Undefined, 8—Luna mammarenavirus, 9—Argentinian mammarenavirus, 10—Machupo mammarenavirus, 11—Wenzhou mammarenavirus, 12—Rat mammarenavirus, 13—Brazilian mammarenavirus, 14—Bear Canyon mammarenavirus, 15—Tamiami mammarenavirus, 16—Ippy mammarenavirus, and 17—Lymphocytic choriomeningitis mammarenavirus.

In the panel of Betainfluenzavirus: 1—Influenza B virus, and 2—Undefined.

In the panel of Norovirus: 1—Norwalk virus, and 2—Undefined.

In the panel of Hepacivirus: 1—Hepacivirus C, 2—Guangxi houndshark hepacivirus, 3—Hepatitis GB virus B, 4—Undefined, 5—Rodent hepacvirus, 6—Equine hepacivirus, 7—Bovine hepacivirus, 8—Hepacivirus sp., 9—Hepacivirus F, 10—Sifaka hepacivirus, 11—Hepacivirus D, 12—Hepacivirus A, 13—Hepacivirus N, 14—Hepacivirus P, and 15—Duck hepacivirus.

In the panel of Deltainfluenzavirus: 1—Recovirus Bangladesh/289/2007, and 2—Undefined.

In the panel of Flavivirus: 1—West Nile virus, 2—Dengue viruses, 3—Spondweni virus, 4—Powassan virus, 5—Calbertado virus, 6—Wesselsbron virus, 7—Long Pine Key virus, 8—Marisma mosquito virus, 9—Phnom Penh bat virus, 10—Israel turkey meningoencephalomyelitis virus, 11—Sokoluk virus, 12—Kedougou virus, 13—Cacipacore virus, 14—Banzi virus, 15—Zika virus, 16—Culex flavivirus, 17—Aedes flavivirus, 18—Nounane virus, 19—Binjari virus, 20—Cell fusing agent virus, 21—Kadam virus, 22—Yellow fever viruses, 23—Koutango virus, 24—Saint Louis encephalitis virus, 25—Japanese encephalitis virus, 26—Omsk hemorrhagic fever virus, 27—Sepik virus, 28—Royal Farm virus, 29—Meaban virus, 30—Aroavirus, 31—Murray Valley encephalitis virus, 32—Kyasanur Forest disease virus, 33—Tick-bome encephalitis virus, 34—Mediterranean Ochlerotatus Flavivirus, 35—Ilheus virus, 36—Mediterranean Culex Flavivirus, 37—Modoc virus group, 38—Tyuleniy virus, 39—Rio Bravo viruse, 40—Uganda S virus, 41—Louping ill virus, 42—Ntaya virus, 43—Saboya virus, 44—Usutu virus, 45—Chaoyang virus, 46—Jugra virus, 47—Langat virus, 48—Yaounde virus, 49—Kokobera virus, 50—Entebbe bat virus, 51—Quang Binh virus, 52—Gadgets Gully virus, 53—Ochlerotatus caspius flavivirus, 54—Tembusu virus, and 55—Undefined.

In the panel of Gammainfluenzavirus: 1—Influenza C virus.

In the panel of Vesivirus: 1—Vesicular exanthema of swine virus, 2—Canine vesivirus, 3—Undefined, and 4—Feline Calicivirus.

In the panel of Pestivirus: 1—Phocoena pestivirus, 2—Pestivirus C, 3—Atypical porcine pestivirus, 4—Pestivirus B, 5—Undefined, 6—Rodent pestivirus, 7—Pestivirus sp., 8—Pestivirus I, 9—Pestivirus F, 10—Pestivirus A, 11—Pestivirus D, and 12—Pestivirus H.

In the panel of Ebolavirus: 1—Zaire ebolavirus, 2—Bundibugyo ebolavirus, 3—Bombali ebolavirus, 4—Undefined, 5—Sudan ebolavirus, 6—Tai Forest ebolavirus, and 7—Reston ebolavirus.

In the panel of Alphacoronavirus: 1—Human coronavirus 229E, 2—Mystacina coronavirus New Zealand/2013, 3—NL63—related bat coronavirus strain BtKYNL63—9b, 4—Miniopterus bat coronavirus HKU8, 5—Porcine epidemic diarrhea virus, 6—Alphacoronavirus 1, 7—Miniopterus bat coronavirus 1, 8—Ferret coronavirus, 9—Human coronavirus NL63, 10—Bat coronavirus HKU10, 11—Lucheng Rn rat coronavirus, 12—Lushi Ml bat coronavirus, 13—Wencheng Sm shrew coronavirus, 14—Swine acute diarrhea syndrome coronavirus, 15—Undefined, 16—Alphacoronavirus sp., and 17—Bat alphacoronavirus.

In the panel of Alphavirus: 1—Middleburg virus, 2—Highlands J virus, 3—Salmon pancreas disease virus, 4—Undefined, 5—Ross River virus, 6—Chikungunya virus, 7—Sindbis virus, 8—Eastern equine encephalitis virus, 9—Western equine encephalitis virus, 10—Barmah Forest virus, 11—Getah virus, 12—Madariaga virus, 13—Aura virus, 14—Ndumu virus, 15—Venezuelan equine encephalitis virus, 16—Semliki Forest virus, 17—Mayaro virus, and 18—Onyong-nyong virus.

In the panel of Marburgvirus: 1—Undefined, and 2—Marburgv marburgirus.

In the panel of Betacoronavirus: 1—Severe acute respiratory syndrome-related coronavirus, 2—Human coronavirus HKU1, 3—Betacoronavirus sp., 4—Pangolin coronavirus, 5—Rousettus bat coronavirus GCCDC1, 6—Pipistrellus bat coronavirus HKU5, 7—Betacoronavirus 1, 8—Middle East respiratory syndrome-related coronavirus, 9—Rabbit coronavirus HKU14, 10—Longquan Rl rat coronavirus, 11—Coronavirus BtRt-BetaCoV/GX2018, 12—Hedgehog coronavirus 1, 13—Rousettus bat coronavirus HKU9, 14—Tylonycteris bat coronavirus HKU4, 15—Longquan Aa mouse coronavirus, 16—Undefined, and 17—Murine coronavirus.

In the panel of Rubivirus: 1—Rubella virus, 2—Rustrela virus, and 3—Undefined.

In the panel of Lyssavirus: 1—European bat 1 lyssavirus, 2—Bokeloh bat lyssavirus, 3—Gannoruwa bat lyssavirus, 4—Duvenhage lyssavirus, 5—Rabies lyssavirus, 6—European bat 2 lyssavirus, 7—Mokola lyssavirus, 8—Australian bat lyssavirus, 9—Lagos bat lyssavirus, 10—Undefined, 11—Irkut lyssavirus, and 12—Frog lyssa-like virus 1.

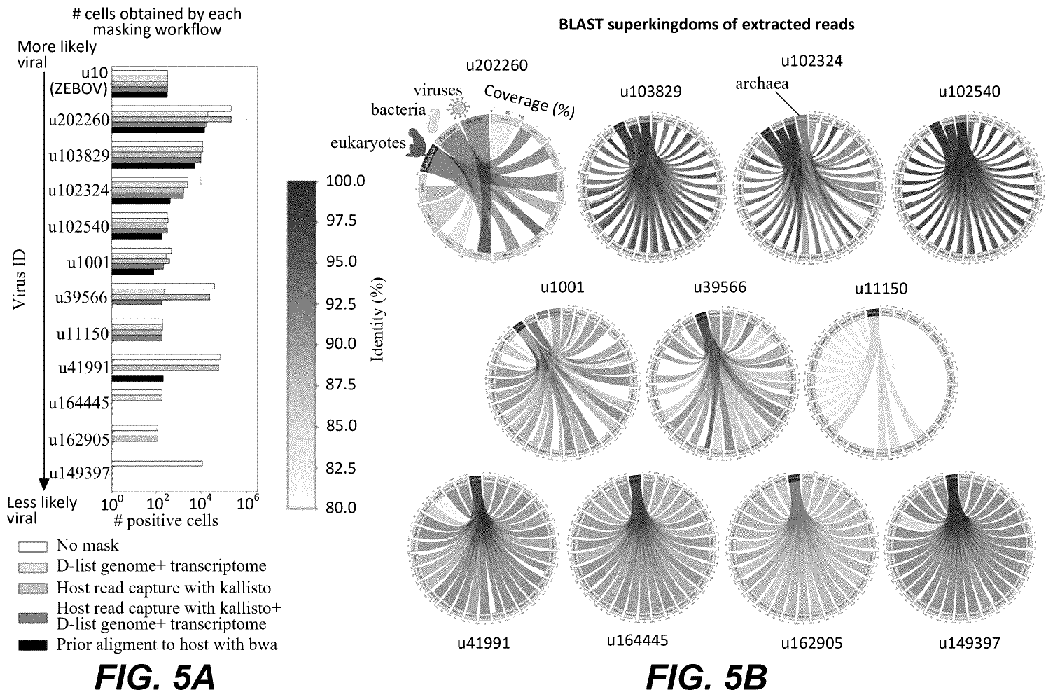

FIG. 15A-FIG. 15B depict non-limiting exemplary embodiments and data related to the presence of viruses in host animals. FIG. 15A depicts the fraction of positive animal (top) and time point (bottom) samples for each virus ID. A sample was considered positive if at least 0.05% of cells were positive. From left to right, the virus ID in FIG. 15A are: u39566, u102540, u11150, u10, u288819, u290519, u10240, u183255, u1001, u100291, u103829, u110641, u181379, u202260, u135858, u101227, u100188, u27694, u34159, u100245, u10015, u100733, u100173, u100196, u100599, u100644, u100296, u100017, u100002, u100012, u100024, u100048, u100302, u100074, u100289, u100026, u100111, u100139, u100154, u100251, u100177, u100215, u100049, u100000, u100001, u100007, u100004, u100011, u100093, u100116, u100019, u100076, u100028, u100153, u100031, u100145, u102324 and u134800. FIG. 15B depicts the number of positive cells for each virus ID or any combination of virus IDs for the count matrices generated from host-masked reads (e.g., D-list host genome and transcriptome+host transcriptome read capture) (left) and reads without any host masking (right). A large amount of reads for u202260 were masked when conservatively removing host reads (FIG. 5A). The plots were generated using PyVenn (github.com/tctianchi/pyvenn).

FIG. 16A-FIG. 16D depict non-limiting exemplary embodiments and data related to the prediction of viral presence by host gene expression. FIG. 16A depicts the average accuracy, specificity, and sensitivity of the logistic regression models trained on highly variable (HV) or all macaque genes with or without donor animal and EVD time point as covariates for the known virus ZEBOV (u10) and 6 novel viruses (top three panels)/5 virus-like sequences (bottom three panels). The logistic regression models were trained to predict the presence of specific viruses based on host gene expression at single-cell resolution. As a negative control, viral presence and absence labels were scrambled at random in the training data. The figure legend for the top three panels is in the left bottom corner of the top panel. The figure legend for the bottom three panels is in the left bottom corner of the fourth panel from top. FIG. 16B depicts weight correlations of the predictive genes (correlations of the average weights of predictive genes) for models trained on HV genes with and without covariates on the real and scrambled labels. The weight correlations are lost when the model is trained using the scrambled labels. Virus ID with high cell type specificity have slightly higher correlations than viruses with low cell type specificity. The color bar indicates the standard deviation (SD) of gene weights generated using different random seeds in the model trained on HV genes with covariates. The weights were max normalized between random seeds before computing the average and SD. FIG. 16C depicts total number of training cells per cell type. The total number consisted of an equal number of virus-positive and -negative cells. FIG. 16D depicts average prediction accuracy of models trained on HV genes with donor animal and EVD time point as covariates for all “macaque only” and “shared” viruses. Error bars indicate the standard deviation between models initialized with different random seeds.

FIG. 17A-FIG. 17F depict non-limiting exemplary embodiments and data related to the predictive genes. FIG. 17A depicts average weight distributions of predictive genes from the models trained on highly variable genes with donor and time point as covariates for the four virus-like sequences with high predictive accuracy. The weights were averaged across models initialized using different random seeds and the standard deviations (SD) of the weights between seeds are shown as the dots. Gene weights were max normalized between random seeds before computing the average and SD. The dashed lines in the top panels indicate the minimum average gene weight and maximum SD for genes included in the enrichment analysis. The dashed lines in the bottom panels show the top 200 gene cut-off for the enrichment analysis. FIG. 17B depicts enrichment analysis of the top 200 predictive genes from the model trained on highly variable genes with donor and time point as covariates. Approximately half of the macaque Ensembl IDs did not have annotated gene names, which is a common problem for genomes from non-model organisms. Gget was used to translate annotated Ensembl IDs to gene symbols and Enrichr to perform enrichment analysis against the GEO microbe perturbations database (e.g., “Microbe_Perturbations_from_GEO up”). Reported P values were corrected with the Benjamini-Hochberg method. In the left panel of FIG. 17B (ZEBOV), 1: Staphylococcus aureus mouse lung . . . ; 2: lymphocytic choriomeningitis . . . ; 3: Lassa virus human peripheral blood . . . ; 4: Mycobacterium tuberculosis . . . ; 5: influenza A mouse spleen, 9 days . . . ; 6: Staphylococcus aureus mouse lung . . . ; 7: Staphylococcus aureus human . . . ; 8: Salmonella enterica serovar . . . ; 9: lymphocytic choriomeningitis . . . ; 10: rhinovirus human bronchial . . . ; 11: Mycobacterium tuberculosis . . . ; 12: H5N1 influenza virus human macrophage . . . ; 13: influenza A mouse spleen, 6 days . . . ; 14: West Nile virus (WNV) human . . . ; 15: Mycobactenum tuberculosis . . . . In the second panel from left of FIG. 17B (u102540), 1: H1N1 influenza virus (pandemic strain) . . . ; 2: H1N1 influenza virus (pandemic strain) . . . ; 3: Mycobacterium tuberculosis . . . ; 4: influenza A mouse blood, 5 days . . . ; 5: Leishmania braziliensis . . . ; 6: rhinovirus human bronchial . . . ; 7: influenza A mouse spleen. 8 days . . . ; 8. HCV human CD4+ T cells GSE49954 . . . ; 9: Staphylococcus aureus human . . . ; 10: influenza virus human whole blood . . . ; 11: Staphylococcus aureus mouse lung . . . ; 12: Leishmania braziliensis . . . ; 13: influenza A mouse blood. 9 days . . . ; 14: influenza A mouse lung, 5 days . . . ; 15: Leishmania braziliensis . . . . In the second panel from right of FIG. 17B (u11150), 1: H5N1 influenza virus human macrophage . . . ; 2: Pseudomonas aeruginosa mouse . . . 3: Coxiella bumetii human monocyte . . . ; 4: Pseudomonas aeruginosa mouse . . . ; 5: Staphylococcus aureus human . . . ; 6: Pseudomonas aeruginosa mouse . . . ; 7: Pseudomonas aeruginosa mouse . . . ; 8: Yersinia enterocolitica . . . ; 9: pandemic influenza H1N1 (pdm H1N1) A . . . ; 10: Staphylococcus aureus mouse lung . . . ; 11: Leishmania major mouse macrophage . . . ; 12: Pseudomonas aeruginosa mouse . . . ; 13: Trypanosoma cruzi human fibroblast . . . ; 14: Shiga toxin type 1 human macrophage . . . ; 15: H1N1 influenza virus (pandemic strain) . . . . In the right panel of FIG. 17B (u202260), 1: H1N1 influenza virus (pandemic strain) . . . ; 2: H1N1 influenza virus (pandemic strain) . . . ; 3: Leishmania amazonensis mouse . . . ; 4: Burkholderia pseudomallei . . . ; 5: lymphocytic choriomeningitis . . . ; 6: Mycobacterium tuberculosis . . . ; 7: Leishmania amazonensis mouse . . . ; 8: lymphocytic choriomenmgitis . . . ; 9: lymphocytic choriomeningitis . . . ; 10: Burkholderia pseudomallei . . . ; 11: influenza virus human whole blood . . . ; 12. HCV human Huh7 GSE20948 microbe: 79; 13: Hepatitis C virus human CD8+ . . . ; 14: respiratory syncytial virus . . . ; 15: respiratory syncytial virus . . . . In FIG. 17B, bars show the number of overlapping genes and dots show the −log₁₀(adjusted P values). The dashed line in FIG. 17B indicates p=0.05. FIG. 17C depicts the prediction of viral presence based on host gene expressing using cell type controlled models. A second round of modeling was performed, whereby virus-negative training cells were selected to be of the same cell types as virus-positive cells. The accuracy, specificity, and sensitivity of these models trained on highly variable (HV) or all macaque genes with donor animal and EVD time point as covariates are shown for the known virus ZEBOV (u10) and 6 novel viruses. As a negative control, viral presence and absence labels were scrambled at random in the training data. FIG. 17D depicts enrichment analysis of predictive genes from the regression model trained on highly variable genes with donor and time point as covariates Approximately one third of the macaque Ensembl IDs did not have annotated gene names, which is a common problem for genomes from non-model organisms. Gget was used to translate annotated Ensembl IDs to gene symbols and Enrichr to perform enrichment analysis against the 2023 Gene Ontology (GO) Biological Processes database (e.g., “GO_Biological_Process_2023”). Gene names are listed on the nght of bar plots. Reported P values were corrected with the Benjamini-Hochberg method. In the left panel of FIG. 17D (ZEBOV), 1: Negative Regulation of Viral Entry into Host Cell (GO: 0046597): 2: Negative Regulation of Viral Life Cycle (GO: 1903901): 3 Regulation of Viral Entry into Host Cell (GO: 0046596); 4: Neuroblast Proliferation (GO: 0007405); 5: Regulation of Establishment of T cell Polarity (GO: 1903903): 6: Positive Regulation of Cysteine-Type Endopeptidase Activity . . . ; 7: Complement Activation. Lectin Pathway (GO: 0001867), 8: Cell Surface Pattern Recognition Receptor Signaling Pathway . . . ; 9: Positive Regulation of Opsonization (GO: 1903028); 10: Regulation of Pososome Assembly (GO: 0071801); 11: Actin-Mediated Cell Contraction (GO: 0070151): 12: Epithelial Cell Apoptotic Process (GO: 1904019): 13: Action Filament Depolymerization (GO: 0030042); 14: Positive Regulation of Actin Nucleation (GO: 0051127); 15: Regulation of Cysteine-Type Endopeptidase Activity involved in . . . ; 16. Regulation of Opsonization (GO: 1903027). In the middle panel of FIG. 17D (u102540), 1: Regulation of Tumor Necrosis Factor Production (GO: 0032680); 2: Negative Regulation of Antigen Receptor-Mediated Signaling Pathway . . . ; 3: Positive Regulation of Type II Interferon Production (GO: (0(32729); 4. Cellular Response to Hypoxia (GO: 0071456): 5: Cellular Response to Decreased Oxygen Levels (GO. 0036294); 6: Positive Regulation of Tumor Necrosis Factor Production (GO: 0032760): 7: Positive Regulation of Cytokine Production (GO 0001819): 8: Positive Regulation of Tumor Necrosis Factor Superfamily Cytokine . . . ; 9: Regulation of Type II Interferon Production (GO: 0032649); 10: Regulation of Cell Cycle Process (GO: 0090068); 11: Negative Regulation of B Cell Receptor Signaling Pathway (GO. 0050829): 12: Maintenance of Protein Location in Extracellular Region (GO: 0071694): 13: Regulation of Extracellular Matrix Assembly (GO: 1901201): 14: Regulation of Cardiac Muscle Hypertrophy in Response to Stress . . . ; 15: Cardiac Atrium Development (GO: 0003230); 16: Regulation of Chromosome Condensation (GO: 0060623). In the right panel of FIG. 17D (u11150), 1: Leukocyte Apoptotic Process (GO: 0071887): 2: p38MAPK Cascade (GO: 0038066): 3: Regulation of Endothelial Cell Development (GO: 1901550); 4: Adherens Junction Assembly (GO: 0034333); 5: Receptor-Mediated Endocytosis of Virus by Host Cell (GO: 0019065): 6: Epithelial Cell-Cell Adhesion (GO: 0090136); 7: Cellular Response to UV-B (GO: 0071493): 8: Cellular Response to Thyroid Hormone Stimulus (GO: 0097067); 9. Response to Thyroid Hormone (GO. 0097066); 10: Positive Regulation of Muscle Cell Differentiation . . . ; 11: Response to UV-B (GO: 0010224); 12. Regulation of Establishment of Endothelial Barrier . . . ; 13: Glycoprotein Catabolic Process (GO: 0006516); 14: Plasma Membrane Bounded Cell Projection Assembly (GO: 0120031); 15: Microtubule Depolymerization (GO: 0007019). 16: Protein Autoprocessing (GO: 0016540). In FIG. 17D, bars show the number of overlapping genes and dots show the −log₁₀(adjusted P values). The dashed line in FIG. 17D indicates p=0.05. FIG. 17E depicts RdRP-like sequences detected in blank sequencing libraries. Sequencing reads were obtained by sequencing multiple ‘blank’ sequencing libranes containing only sterile water and reagent mix. The plot shows the fraction of reads that map to different virus IDs for each sequencing technology. The total number of reads obtaining using different sequencing technology were 11,872,733 (Illumina Novaseq 6000), 741,323 (Illumina NextSeq 500) and 85,348 (Illumina Miseq 150). The fractions were normalized to the total number of reads obtained for each technology. The data was generated by Porter et al. and analyzed using kallisto translated search with PalmDB. FIG. 17F depicts total number of training cells per cell type in the model shown in FIG. 17C. The total number consisted of an equal number of virus-positive and virus-negative cells.

FIG. 18A-FIG. 18E depict non-limiting exemplary embodiments and data related to biochemical and taxonomic features of the comma-free codes in the translated search disclosed herein. FIG. 18A depicts hamming distances between amino acids in the comma-free code (left) and a second code that maximizes Hamming distances between amino acids that occur most often (right). FIG. 18B depicts expected and observed counts per sOTU in two experiments. All amino acid sequences in the PalmDB were reverse translated using the “standard” genetic code. The reverse translated PalmDB RdRP sequences were subsequently aligned to the optimized PalmDB amino acid reference with kallisto translated search. The left plots show the expected and observed counts for each sOTU when kallisto performs the pseudoalignment in the comma-free code space. The plots on the right show the expected and observed counts for each sOTU when kallisto performs the pseudoalignment using a second code that maximized the Hamming distances between reverse translated amino acids. FIG. 18C depicts occurrence of each amino acid in the PalmDB. FIG. 18D depicts percentage of differing amino acids or nucleotides between 10,000 sequences randomly selected from the PalmDB before and after reverse translation using the standard genetic code (optimized for human) and comma-free code. FIG. 18E depicts the virus orders of RdRP sequences sorted based on their clustering by MMseqs.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the terms “silent nucleotide mutation” and “silent mutation” are interchangeable and refer to a change in nucleic acid sequence that doesn't alter the amino acid sequence of a protein encoded by the nucleic acid sequence.

As used herein, the term “comma-free code” refers to a nucleic acid sequence that doesn't require spaces or commas to indicate codon boundaries. Triplet codons are “sense” if they correspond to an amino acid and are “non-sense” if they do not correspond to an amino acid. A nucleic acid sequence can have multiple reading frames, which is known as frameshifting. For example, a single-strand nucleic acid can have three reading frames, while a double-strand DNA can have 6 reading frames. If only one reading frame of a nucleic acid sequence contains sense codon and all the triplet codons in other reading frame of the nucleic acid sequence are nonsense, then the nucleic acid sequence is comma-free, because the message contained in the nucleic acid sequence has only one reading. A code with this property is said to be comma-free, since messages remain unambiguous even when words are run together without commas or spaces. In some embodiments, the nucleic acid is double-strand DNA and both strands of the double-strand DNA are comma-free. The strong property of such codes is the immediate detection of the wrong reading frame.

As used herein, the terms “conserved sequence” and “conservative sequence” are interchangeable and can refer to a nucleic acid sequence (e.g., DNA or RNA) or an amino acid sequence with high similarity/identity across different species. In some embodiments, the conserved sequence maintains at least 50% (e.g., 50%, 55%, 60%, 65%, 70%, 75%, 80%, 85%, 90%, 95% or 100%) similarity/identity across different species. In some embodiment, the conserved sequence is a nucleic acid encoding and/or is the amino acid sequence of RNA-dependent RNA polymerase (RdRp).

As used herein, the terms “nucleic acid” and “polynucleotide” are interchangeable and can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

The terms “nucleic acid” and “polynucleotide” also specifically include nucleic acids composed of bases other than the five biologically occurring bases (adenine, guanine, thymine, cytosine and uracil).

As used herein, the terms “comma-free code space” and “comma-free space” are interchangeable and refer to a collection of nucleic acid sequences that are all comma-free.

As used herein, the term “amino acid space” refers to a collection of amino acid sequences.

As used herein, the term “nucleotide space” to a collection of nucleic acid sequences. The nucleic acid sequences can comprise sequences that are comma-free, not comma-free, or both.

As used herein, the terms “multimapped” and “multimapping” are interchangeable and refer to the situation that a sequence (e.g., amino acid sequence or nucleic acid sequence) aligned to multiple targets in the reference (e.g., reference amino acid sequence or reference nucleic acid sequence) and could not unambiguously be assigned to one.

As used herein, the term “host sequence” refers to nucleic acid sequences in a host cell that is not infected by microbes. The nucleic acid sequences in a host cell can be host genome or host transcriptome.

A method that accurately and rapidly detected viral sequences in bulk and single-cell transcriptomic data based on highly conserved amino acid domains is disclosed herein, which enabled the detection of RNA viruses covering at least 100,000 (e.g., 146,973) virus species. The analysis of viral presence and host gene expression in parallel at single-cell resolution allowed for the characterization of host viromes and the identification of viral tropism and host responses. By applying the method disclosed herein, novel viruses were identified in rhesus macaque PBMC data that displayed cell type specificity and whose presence correlated with altered host gene expression.

Disclosed herein include methods for detecting microbes in a sample. In some embodiments, the method comprises: converting a plurality of reference sequences to a plurality of comma-free reference codes; converting a plurality of sample sequences to a plurality of comma-free sample codes; and aligning the plurality of comma-free reference codes to the plurality of comma-free sample codes to generate a microbe profile of the sample, thereby detecting the presence of one or more microbes in the sample.

Disclosed herein include methods for predicting or detecting microbes in a sample. The method can comprise: providing a model with a training dataset to determine a weight of each gene in the training data, wherein the model is a logistic regression modal, and wherein the training dataset comprises sequencing data of one or more cells; determining one or more signature genes, wherein the signature genes have weights no less than a threshold; providing a trained model with a testing dataset, wherein the trained model is parameterized with the weight of the signature genes and wherein the testing dataset comprises sequencing data of one or more cells in the sample; and determining a probability of presence of the microbes using the trained model, thereby determining the presence or absence of the microbes in the sample.

### Virus and Viral “Hallmark” Sequences

There are an estimated 10³¹ virions on Earth, among which more than 300,000 virus species are estimated to cause human disease. However, only 261 species have been detected in humans. Of the 261 known disease-causing viruses, 206 fall into the realm of Riboviri. In some embodiments, the virus detected using the methods disclosed herein is a virus from the realm of Riboviria. Examples of diseases-causing viruses in the realm of Riboviri include Coronaviruses, Dengue viruses, Ebolaviruses, Hepatitis B viruses, influenza viruses, Measles viruses, Mumps viruses, Polio viruses, West Nile viruses, and Zika viruses. Coronaviruses are enveloped positive sense RNA viruses ranging from 60 nm to 140 nm in diameter with spike like projections on its surface giving it a crown like appearance under the electron microscope; hence the name coronavirus. In some embodiments the coronaviruses are alphacoronavirus (e.g., human coronavirus 229E, mystacina coronavirus New Zealand/2013, NL63-related bat coronavirus strain BtKYNL63-9b, miniopterus bat coronavirus HKU8, porcine epidemic diarrhea virus, alphacoronavirus 1, miniopterus bat coronavirus 1, ferret coronavirus, human coronavirus NL63, bat coronavirus HKU10, Lucheng Rn rat coronavirus, Lushi Ml bat coronavirus, Wencheng Sm shrew coronavirus, swine acute diarrhea syndrome coronavirus, alphacoronavirus sp., and bat alphacoronavirus), betacoronavirus (e.g., severe acute respiratory syndrome-related coronavirus, human coronavirus HKU1, betacoronavirus sp., pangolin coronavirus, rousettus bat coronavirus GCCDC1, Pipistrellus bat coronavirus HKU5, betacoronavirus 1, Middle East respiratory syndrome-related coronavirus, rabbit coronavirus HKU14, Longquan Rl rat coronavirus, coronavirus BtRt-BetaCoV/GX2018, hedgehog coronavirus 1, rousettus bat coronavirus HKU9, Tylonycteris bat coronavirus HKU4, Longquan Aa mouse coronavirus, and murine coronaviru), deltacoronavirus (e.g., sparrow deltacoronavirus, coronavirus HKU15 and quail coronavirus UAE-HKU30), and gammacoronavirus (e.g., avian coronavirus and beluga whale coronavirus SW1). Ebola virus (EBOV) belongs to the family Filoviridae, the genus Ebolavirus, and frequently causes fatal infection in humans. The EBOV genome is a single negative-sensed RNA, with genome size of 19 Kb. Examples of EBOV include Zaire ebolavirus, Bundibugyo ebolavirus, Bombali ebolavirus, Sudan ebolavirus, Tai Forest ebolavirus and Reston ebolavirus. In some embodiments, the viruses that can be detected using the method disclosed herein are viruses listed in Table 3.

In some embodiments, the virus is Duplomaviricota, Kitrinoviricota, Lenarviricota, Negamaviricota, Peploviricota and Fusariviridae. In some embodiments, the virus is selected from the group consisting of coronaviruses, dengue viruses, ebolaviruses, hepatitis B viruses, influenza viruses, measles viruses, mumps viruses, polioviruses, West Nile viruses and Zika viruses. In some embodiments, the virus detected using the methods disclosed herein include orthoreovirus (e.g., piscine orthoreovirus, piscine orthoreovirus 3, mammalian orthoreovirus, avian orthoreovirus, and pteropine orthoreovirus), deltacoronavirus (e.g., sparrow deltacoronavirus, coronavirus HKU15 and quail coronavirus UAE-HKU30), arterivirus (e.g., betaarterivirus suid 2, deltaarterivirus pejah, etaarterivirus ugarco 1, epsilonarterivirus safriver, deltaarterivirus hemfev, kappaarterivirus wobum, thetaarterivirus mikelba 1, alphaarterivirus equid and gammaarterivirus lacdeh), rotavirus (e.g., rotavirus I, rotavirus C, rotavirus H, rotavirus F, murine rotavirus, rotavirus D, tasmanian devil-associated rotavirus 1, rotavirus A, rotavirus B and rotavirus G), gammacoronavirus (e.g., avian coronavirus and beluga whale coronavirus SW1), morbillivirus (e.g., measles morbillivirus, feline morbillivirus, canine morbillivirus, rinderpest morbillivirus, small ruminant morbillivirus, cetacean morbillivirus, phocine morbillivirus and feline morbillivirus type 2), cardiovirus (e.g., cardiovirus A, cardiovirus B and cardiovirus C), orthohepevirus (e.g., orthohepevirus A, orthohepevirus B and orthohepevirus C), enterovirus (e.g., avian metapneumovirus and human metapneumovirus), metapneumovirus (e.g., enterovirus A, enterovirus C, enterovirus D, enterovirus B, rhinovirus C, enterovirus J, enterovirus G, human rhinovirus sp., goat enterovirus, enterovirus E, enterovirus F, enterovirus sp., enterovirus H, rhinovirus A and rhinovirus B), piscihepevirus (e.g., piscihepevirus A), respirovirus (e.g., human respirovirus 1, murine respirovirus, bovine respirovirus 3, human respirovirus 3 and porcine respirovirus 1), hepatovirus (e.g., hedgehog hepatovirus and hepatovirus A), arenavirus (e.g., mopeia lassa virus reassortant 29), alphainfluenzavirus (e.g., influenza A virus), sapelovirus (e.g., sapelovirus A and Sapelovirus B), mammarenavirus (e.g., guanarito mammarenavirus, lujo mammarenavirus, cali mammarenavirus, tacaribe mammarenavirus, pirital mammarenavirus, lassa mammarenavirus, luna mammarenavirus, argentinian mammarenavirus, machupo mammarenavirus, wenzhou mammarenavirus, rat mammarenavirus, brazilian mammarenavirus, bear canyon mammarenavirus, tamiami mammarenavirus, ippy mammarenavirus and lymphocytic choriomeningitis mammarenavirus), betainfluenzavirus (e.g., influenza B virus), norovirus (e.g., norwalk virus), hepacivirus (e.g., hepacivirus C, Guangxi houndshark hepacivirus, hepatitis GB virus B, rodent hepacvirus, equine hepacivirus, bovine hepacivirus, hepacivirus sp., hepacivirus F, sifaka hepacivirus, hepacivirus D, hepacivirus A, hepacivirus N, hepacivirus P and duck hepacivirus), deltainfluenzavirus (e.g., recovirus bangladesh/289/2007), flavivirus (e.g., West Nile virus, dengue viruses, spondweni virus, powassan virus, calbertado virus, wesselsbron virus, long pine key virus, marisma mosquito virus, phnom penh bat virus, Israel turkey meningoencephalomyelitis virus, sokoluk virus, kedougou virus, cacipacore virus, banzi virus, Zika virus, Culex flavivirus, Aedes flavivirus, nounane virus, binjari virus, cell fusing agent virus, kadam virus, yellow fever viruses, koutango virus, Saint Louis encephalitis virus, Japanese encephalitis virus, omsk hemorrhagic fever virus, sepik virus, royal farm virus, meaban virus, aroa virus, Murray Valley encephalitis virus, kyasanur forest disease virus, tick-borne encephalitis virus, mediterranean ochlerotatus flavivirus, ilheus virus, mediterranean Culex flavivirus, modoc virus group, tyuleniy virus, rio bravo viruse, Uganda S virus, louping ill virus, ntaya virus, saboya virus, usutu virus, chaoyang virus, jugra virus, langat virus, yaounde virus, kokobera virus, entebbe bat virus, quang binh virus, gadgets gully virus, ochlerotatus caspius flavivirus and tembusu virus), gammainfluenzavirus (e.g., influenza C virus), vesivirus (e.g., vesicular exanthema of swine virus, canine vesivirus and feline calicivirus), pestivirus (e.g., phocoena pestivirus, pestivirus C, atypical porcine pestivirus, pestivirus B, rodent pestivirus, pestivirus sp., pestivirus I, pestivirus F, pestivirus A and pestivirus H), ebolavirus (e.g., Zaire ebolavirus, bundibugyo ebolavirus, bombali ebolavirus, Sudan ebolavirus, Tai Forest ebolavirus and reston ebolavirus), alphacoronavirus (e.g., human coronavirus 229E, mystacina coronavirus New Zealand/2013, NL63-related bat coronavirus strain BtKYNL63-9b, miniopterus bat coronavirus HKU8, porcine epidemic diarrhea virus, alphacoronavirus 1, miniopterus bat coronavirus 1, ferret coronavirus, human coronavirus NL63, bat coronavirus HKU10, lucheng Rn rat coronavirus, lushi Ml bat coronavirus, wencheng Sm shrew coronavirus, swine acute diarrhea syndrome coronavirus, alphacoronavirus sp. and bat alphacoronavirus), alphavirus (e.g., Middleburg virus, Highlands J virus, salmon pancreas disease virus, Ross River virus, chikungunya virus, sindbis virus, eastern equine encephalitis virus, western equine encephalitis virus, Barmah Forest virus, getah virus, madariaga virus, aura virus, ndumu virus, venezuelan equine encephalitis virus, semliki forest virus, mayaro virus and onyong-nyong virus), marburgvirus (e.g., marburgv marburgirus), betacoronavirus (e.g., severe acute respiratory syndrome-related coronavirus, human coronavirus HKU1, betacoronavirus sp., pangolin coronavirus, rousettus bat coronavirus GCCDC1, Pipistrellus bat coronavirus HKU5, betacoronavirus 1, Middle East respiratory syndrome-related coronavirus, rabbit coronavirus HKU14, longquan R1 rat coronavirus, coronavirus BtRt-BetaCoV/GX2018, hedgehog coronavirus 1, rousettus bat coronavirus HKU9, Tylonycteris bat coronavirus HKU4, longquan Aa mouse coronavirus and murine coronavirus), rubivirus (e.g., rubella virus and rustrela virus), lyssavirus (e.g., European bat 1 lyssavirus, bokeloh bat lyssavirus, gannoruwa bat lyssavirus, duvenhage lyssavirus, rabies lyssavirus, European bat 2 lyssavirus, mokola lyssavirus, Australian bat lyssavirus, lagos bat lyssavirus, irkut lyssavirus, or frog lyssa-like virus 1.

Riboviria is the first realm created to group all viruses with RNA genomes. These RNA viruses encode either an RdRp or a reverse transcriptase (e.g., RNA-dependent DNA polymerase (RdDp)).

### RNA-Dependent RNA Polymerase (RdRp)

The viral polymerase (e.g., RdRp and RdDp) fold belongs to the template-dependent nucleic acid polymerase superfamily, which resembles a grasping right hand with thumb contacting finger. Although amino acid identity of the polymerase is low (e.g., as low as 10%) between diverged species, surface regions of the viral polymerase directly involved in nucleotide selection or catalysis are strongly conserved, in particular short motifs conventionally designated by letters A through G found in the active site. For example, motifs A, B and C found in the palm sub-domain are well conserved in most known RdRPs. The core RdRp domain consists of the thumb, palm and the fingers sub-domains that are primarily involved in template binding, polymerization, nucleoside triphosphate (NTP) entry and associated functions. The palm sub-domain is at the junction of the fingers and the thumb subdomains and houses most of the structurally conserved elements involved in catalysis. The catalytic aspartates and the RNA Recognizing Motif (RRM) comprising three β-strands are present in the palm subdomain. The sub-domain selects NTPs over deoxy NTPs and catalyzes the phosphoryl transfer reaction by coordinating two metal ions (e.g., Mg+/Mn+ cation). Motifs A and C contain essential aspartic acid residues, which coordinate the Mg+/Mn+ cation for catalysing phosphodiester bond formation, while motif B contains an almost perfectly conserved glycine required for nucleotide selection. The motifs appear in ABC (canonical) order in the primary sequence of most known polymerases, but the active site sequence is permuted into CAB order in several independent lineages.

### RNA-Dependent DNA Polymerase (RdDp)

RNA-dependent DNA polymerases (RdDp) are reverse transcriptase (RT) also exhibit conserved structural domains. Some of these domains are shared with other families of nucleic acid polymerases. During the polymerization process, the protein binds to the single-stranded RNA template molecule and synthesizes a complementary DNA molecule. After synthesis, an RNA-DNA heteroduplex is formed. Besides the catalytic domain, RdDps have an exonuclease domain, which is used to degrade the RNA molecule from the heteroduplex. From the single-stranded DNA molecule, the complementary DNA strand is then synthesized, resulting in a double-stranded DNA molecule at the end of the process. Observing this process, RdDp is expected to also exhibit DNA-dependent DNA polymerase activity. The structural of RdDp in some viruses has been studied. For example, in HIV type 1 (HIV-1), RT is a multifunctional heterodimeric enzyme composed of subunits of 66 and 51 kDa (p66/p51), with DNA polymerase and ribonuclease H (RNase H) activities. For DNA polymerization, RTs can use as templates either RNA (RNA-dependent DNA polymerase (RdDp)) or DNA (DNA-dependent DNA polymerase (DDDP)). DNA polymerase and RNase H activities are both essential for viral replication, and are located in two separated domains of the p66 RT subunit. The DNA polymerase domain is located at the N-terminus and exhibits the classical “right hand” conformation, while the RNase H domain is located at the C-terminus, 60 Å away from the polymerase active site. The distance between the active sites of the polymerase and the RNase H is estimated at around 17-18 base pairs, and both domains are linked by a so-called connection subdomain. Long-range effects and functional interdependence between active domains are been suggested, based on mutational studies showing that residues such as Pro226, Phe227, Gly231, Tyr232, Glu233, and His235 at the polymerase domain of the HIV-1 RT could affect RNase H activity, whereas deletions at the C-terminus can decrease the efficiency of DNA polymerization.

Due to the wide-spreading of these genes encoding key components and conservativeness of at least some domain/motifs in these genes, these genes can be “hallmark genes” used for the identification of viruses. In some embodiment, the reference sequences comprise the hallmark genes. In some embodiment, the reference sequences comprise the amino acid sequences of RdRp and RdDp. In some embodiment, the reference sequences comprise the nucleic acid sequences encoding RdRp and RdDp. However, RNA viruses have highly divergent sequences, even within the conserved RdRP. Some researches show that amino acid sequence alignment can recover the majority of RdRP short reads above 60% identity. Thus, in some embodiments, the references sequences comprise the hallmark sequence. The hallmark sequence can be a conserved region within a gene or a non-gene sequence. In some embodiments, the reference sequences comprise the amino acid sequence of a catalytic domain (e.g., palm sub-domain of RdRp). In some embodiments, the reference sequences comprise the amino acid sequences of several catalytic domains in a conserved protein. In some embodiments, the reference sequences comprise the nucleic acid sequence encoding a catalytic domain (e.g., palm sub-domain of RdRp). In some embodiments, the reference sequences comprise the nucleic acid sequence encoding several catalytic domains in a conserved protein. In some embodiments, the reference sequences or hallmark sequences are about or at least about 60% (e.g., 60%, 65%, 70%, 75%, 80%, 85%, 90%, 95% or 100%) identical across viral species (e.g., viruses in the realm of Riboviria).

In some embodiments, the methods disclosed herein is used to identify therapeutic sequences. In some embodiments, the therapeutic sequences are amino acid sequences of and/or nucleic acid sequences encoding antimicrobial peptides. To identify therapeutic sequences, the reference sequences can be known therapeutic sequences (e.g., amino acid sequences of antimicrobial peptides). The amino acid sequences of antimicrobial peptides can be from databases, such as Database of Antimicrobial Activity and Structure of Peptides (DBAASP), LAMP2, dbAMP, PlantPepDB, starPepDB and ADAPTABLE. In some embodiments, the method disclosed herein is used to identify microbes (e.g., bacteria). The microbe can be bacteria in microbiome of a host (e.g., human gut microbiome). To identify bacteria in microbiome of a host, the reference sequences can be derived from a 16S rRNA database.

In some embodiments, the number of microbe species (e.g., viral species) that can be identified with the method disclosed herein is about or at least about 8,000 species (e.g., 8,000 species, 9,000 species, 10,000 species, 11,000 species, 12,000 species, 13,000 species, 14,000 species, 15,000 species, 20,000 species, 25,000 species, 30,000 species, 35,000 species, 40,000 species, 45,000 species, 50,000 species, 60,000 species, 70,000 species, 80,000 species, 90,000 species, 100,000 species, 110,000 species, 120,000 species, 130,000 species, 140,000 species, 150,000 species, 160,000 species, 170,000 species, 180,000 species, 190,000 species, 200,000 species, 300,000 species, 400,000 species, 500,000 species, 600,000 species, 700,000 species, 800,000 species, 900,000 species or 1,000,000 species). In some embodiments, the number of microbe species that can be identified is about or at least about 100,000 (e.g., 146,973).

### Comma-Free Code

To perform the alignment disclosed herein, the sample sequences and the reference sequences needs to be in a “shared” language. For example, the reference sequences can comprise amino acid sequences, while the sample sequences comprise nucleic acid sequences, which cannot be aligned with the reference sequences directly. Thus, in this example, one of the following conversions need to be conducted: 1) translate the sample sequences into amino acid sequences; 2) reverse translate the reference sequences to nucleic acid sequences; or 3) translate both the sample sequences and the reference sequences to another genetic code. Such genetic code can be comma-free code, circular code or a code maximizing Hamming distance between frequently occurring amino acids. Hamming distance is a metric for comparing two binary data strings. While comparing two binary strings of equal length, Hamming distance is the number of bit positions in which the two bits are different. In the context of nucleic acid sequences and amino acid sequences, the Hamming distance compares how different two nucleic acid sequences and amino acid sequences. Thus, methods of calculating the Hamming distance between two nucleic acid sequences/amino acid sequences are known in the field and can comprise converting the nucleic acid sequences/amino acid sequences to binary strings.

In some embodiments, the methods disclosed herein convert reference sequences and sample sequences to codes that have only one reading. In some embodiment, the codes are comma-free codes. In some embodiment, the codes are circular and/or strong comma-free codes. A comma free code has only one correct reading frame. A comma-free code consists of only one permutation of a nucleotide combination. For example, given the nucleotide combination ATCC and its permutations CATC, CCAT and TCCA, only one of these permutations would be included in a comma-free code.

Comma-free codes constitute a class of circular codes, which has also been widely studied. The circular code theory initiated in 1996 proposes that genes are based on a circular code of 20 trinucleotides for retrieving, maintaining and synchronizing the reading frame as well as for coding amino acids. A trinucleotide circular code has the fundamental property to always retrieve the reading frame in any position of any sequence generated with the circular code. In particular, initiation and stop trinucleotides as well as any frame signals are not necessary to define the reading frame. Indeed, a window of a few nucleotides, whose nucleotide length depends on the class of circular codes, positioned anywhere in a sequence generated with the circular code always retrieves the reading frame. The combinatorial properties of comma-free codes and circular codes are important to understand some properties of the genetic code and its encoded amino acids as well as its evolution. Based on a recent approach using graph theory to study circular codes, a new class of circular codes, called strong comma-free codes, is identified. The class of strong comma-free codes is a proper subclass of the class of comma-free codes. The advantage of strong comma-free codes is that two consecutive nucleotides suffice for retrieving the correct reading frame in any sequence generated by the code.

Methods of generating comma-free code is known in the field. For example, comma-free code can be generated using binary templates as described in M. Arita, S. Kobayashi, DNA sequence design using templates, New Gener. Comput. 20 (3) (2002) 263-278; S. Kobayashi, T. Kondo, M. Arita, On template method for DNA sequence design, DNA8, Lecture Notes in Computer Science, vol. 2568, Springer, Berlin, 2002, pp. 205-214; and King, Oliver D., and Philippe Gaborit. “Binary templates for comma-free DNA codes.” Discrete Applied Mathematics 155.6-7 (2007): 831-839, which are incorporated by reference by their entirety.

### Anti-Viral Immune Responses

A tightly coordinated immune response is usually observed during viral infection and is critical to protect the host during viral infections. Studies have shown that natural killer (NK) cells contribute to early anti-viral defenses by exerting antiviral effects through the secretion of interferon (IFN)-γ and by elimination of virus-infected cells. Antigen-specific immune responses mounted by T cells, particularly effector CD8 T cells, and B cells are required to mediate sustained anti-viral resistance and clearance of virus-infected cells. All of these responses are initiated and regulated through the action of the innate immune response (the body's first line of defense). The innate immune system, also known as non-specific (or unspecific) immune system, typically comprises the cells and mechanisms that defend the host from infection by other organisms in a non-specific manner. Cells of the innate immune system express a variety of germ-line encoded pattern recognition receptors which function to sense viral products, induce anti-viral effectors, and initiate adaptive immunity. Of these, toll-like receptors 3, 7, and 9 recognize internalized DNA and RNA viruses in endosomes, TLR4 recognizes certain viral proteins, while the RNA helicase receptors, RIG-I and MDA5 discriminate between distinct classes of RNA viruses in the cytoplasm. In some embodiments, the effectors involved in the innate immune response include: TNF-alpha, CD40, cytokines, monokines, lymphokines, interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33), chemokines, interferons (e.g., IFN-alpha, IFN-beta and IFN-gamma), GM-CSF, G-CSF, M-CSF, LT-beta, growth factors, hGH, Toll-like receptors (e.g., TLR1, TLR2, TLR3, TLR4, TLR5, TLR6, TLR7, TLRB, TLR9, TLR10, TLR11, TLR12 and TLR13), NOD-like receptors, RIG-1 like receptors, immunostimulatory nucleic acids, an immunostimulatory RNA (isRNA) and CpG-DNAs. Growing evidence also indicates the importance of cytosolic DNA sensing mechanisms in anti-viral defenses. The sensing of viruses by innate receptors triggers type I IFNs, the earliest of the anti-viral defense strategies, which act at multiple levels to regulate anti-viral resistance and modulate the activity of other immune cells. Type I IFNs are not the only key innate effector response turned on by these pathways however, stimulation of virus sensing pathways also lead to the expression of pro-inflammatory cytokines including Interleukin IL-1 and IL-18 that also contribute to the clearance of viruses at multiple levels.

In some embodiments, proteins involved in host response to viral infection comprise: ARFGAP1, ARFGAP2, ARFGAP3, ARFGEF1, ARFGEF2, ARFGEF3, CCR1, CCR10, CCR2, CCR3, CCR4, CCR5, CCR6, CCR7, CCR8, CCR9, CCRL2, CCS, CCSAP, CCSER1, CCSER2, CCT2, CCT3, CCT4, CCT5, CCT6A, CCT6B, CCT7, CCT8, CCT8L2, CCZ1, CCZ1B, CD101, CD109, CD14, CD151, CD160, CD163, CD163L1, CD164, CD164L2, CD177, CD180, CD19, CD1A, CD1B, CD1C, CD1D, CD1E, CD2, CD200, CD200R1, CD200R1L, CD207, CD209, CD22, CD226, CD24, CD244, CD247, CD248, CD27, CD274, CD276, CD28, CD2AP, CD2BP2, CD300A, CD300C, CD300E, CD300LB, CD300LD, CD300LF, CD300LG, CD302, CD320, CD33, CD34, CD36, CD37, CD38, CD3D, CD3E, CD3EAP, CD3G, CD4, CD40, CD40LG, CD44, CD46, CD47, CD48, CD5, CD52, CD53, CD55, CD58, CD59, CD5L, CD6, CD63, CD68, CD69, CD7, CD70, CD72, CD74, CD79A, CD79B, CD80, CD81, CD82, CD83, CD84, CD86, CD8A, CD8B, CD9, CD93, CD96, CD99, CD99L2, CDA, CDADC1, CDAN1, CDC123, CDC14A, CDC14B, CDC16, CDC20, CDC20B, CDC23, CDC25A, CDC25B, CDC25C, CDC26, CDC27, CDC34, CDC37, CDC37L1, CDC40, CDC42, CDC42BPA, CDC42BPB, CDC42BPG, CDC42EP1, CDC42EP2, CDC42EP3, CDC42EP4, CDC42EP5, CDC42SE1, CDC42SE2, CDC45, CDC5L, CDC6, CDC7, CDC73, CDCA2, CDCA3, CDCA4, CDCA5, CDCA7, CDCA7L, CDCA8, CDCP1, CDCP2, CDH1, CDH10, CDH11, CDH12, CDH13, CDH15, CDH16, CDH17, CDH18, CDH19, CDH2, CDH2O, CDH22, CDH23, CDH24, CDH26, CDH3, CDH4, CDH5, CDH6, CDH7, CDH8, CDH9, CDHR1, CDHR2, CDHR3, CDHR4, CDHR5, CDIP1, CDIPT, CDK1, CDK10, CDK11A, CDK11B, CDK12, CDK13, CDK14, CDK15, CDK16, CDK17, CDK18, CDK19, CDK2, CDK20, CDK2AP1, CDK2AP2, CDK3, CDK4, CDK5, CDK5R1, CDK5R2, CDK5RAP1, CDK5RAP2, CDK5RAP3, CDK6, CDK7, CDK8, CDK9, CDKAL1, CDKL1, CDKL2, CDKL3, CDKL4, CDKL5, CDKN1A, CDKN1B, CDKN1C, CDKN2A, CDKN2AIP, CDKN2AIPNL, CDKN2B, CDKN2C, CDKN2D, CDKN3, CDNF, CDO1, CDON, CDPF1, CDR1, CDR2, CDR2L, CDRT1, CDRT15, CDRT15L2, CDRT4, CDS1, CDS2, CDSN, CDT1, CDV3, CDX1, CDX2, CDX4, CDY1, CDY1B, CDY2A, CDY2B, CDYL, CDYL2, CTSA, CTSB, CTSC, CTSD, CTSE, CTSF, CTSG, CTSH, CTSK, CTSL, CTSO, CTSS, CTSV, CTSW, CTSZ, DAND5, EMILIN1, EMILIN2, EMILIN3, EML1, EML2, EML3, EML4, EML5, EML6, FCAR, FCER1A, FCER1G, FCER2, FCF1, FCGBP, FCGR1A, FCGR1B, FCGR2A, FCGR2B, FCGR2C, FCGR3A, FCGR3B, FCGRT, FCHO1, FCHO2, FCHSD1, FCHSD2, FCMR, FCN1, FCN2, FCN3, FCRL1, FCRL2, FCRL3, FCRL4, FCRL5, FCRL6, FCRLA, FCRLB, GSDMA, GSDMB, GSDMC, GSDMD, GSE1, GSG1, GSG1L, GSG1L2, GSK3A, GSK3B, GSKIP, GSN, GSPT1, GSPT2, GSR, GSS, GSTA1, GSTA2, GSTA3, GSTA4, GSTA5, GSTCD, GSTK1, GSTM1, GSTM2, GSTM3, GSTM4, GSTM5, GSTO1, GSTO2, GSTP1, GSTT1, GSTT2, GSTT2B, GSTTP1, GSTZ1, IL10, IL10RA, IL10RB, IL11, IL11RA, IL12A, IL12B, IL12RB1, IL12RB2, IL13, IL13RA1, IL13RA2, IL15, IL15RA, IL16, IL17A, IL17B, IL17C, IL17D, IL17F, IL17RA, IL17RB, IL17RC, IL17RD, IL17RE, IL17REL, IL18, IL18BP, IL18R1, IL18RAP, IL19, IL1A, IL1B, IL1F10, IL1R1, IL1R2, IL1RAP, IL1RAPL1, IL1RAPL2, IL1RL1, IL1RL2, IL1RN, IL2, IL20, IL20RA, IL20RB, IL21, IL21R, IL22, IL22RA1, IL22RA2, IL23A, IL23R, IL24, IL25, IL26, IL27, IL27RA, IL2RA, IL2RB, IL2RG, IL3, IL31, IL31RA, IL32, IL33, IL34, IL36A, IL36B, IL36G, IL36RN, IL37, IL3RA, IL4, IL4I1, IL4R, IL5, IL5RA, IL6, IL6R, IL6ST, IL7, IL7R, IL9, IL9R, ILDR1, ILDR2, ILF2, ILF3, ILK, ILKAP, ILVBL, IREB2, IRF1, IRF2, IRF2BP1, IRF2BP2, IRF2BPL, IRF3, IRF4, IRF5, IRF6, IRF7, IRF8, IRF9, IRGC, IRGM, IRGQ, IRS1, IRS2, IRS4, IRX1, IRX2, IRX3, IRX4, IRX5, IRX6, KIF11, KIF12, KIF13A, KIF13B, KIF14, KIF15, KIF16B, KIF17, KIF18A, KIF18B, KIF19, KIF1A, KIF1B, KIF1BP, KIF1C, KIF20A, KIF20B, KIF21A, KIF21B, KIF22, KIF23, KIF24, KIF25, KIF26A, KIF26B, KIF27, KIF2A, KIF2B, KIF2C, KIF3A, KIF3B, KIF3C, KIF4A, KIF4B, KIF5A, KIF5B, KIF5C, KIF6, KIF7, KIF9, KIFAP3, KIFC1, KIFC2, KIFC3, LMNA, LMNB1, LMNB2, LMNTD1, LMNTD2, MAPK1, MAPK10, MAPKTT, MAPK12, MAPK13, MAPK14, MAPK15, MAPK1IPTL, MAPK3, MAPK4, MAPK6, MAPK7, MAPK8, MAPK8IP1, MAPK8IP2, MAPK8IP3, MAPK9, MAPKAP1, MAPKAPK2, MAPKAPK3, MAPKAPK5, MAPKBP1, MS4A1, MS4A10, MS4A12, MS4A13, MS4A14, MS4A15, MS4A2, MS4A3, MS4A4A, MS4A4E, MS4A5, MS4A6A, MS4A6E, MS4A7, MS4A8, MZB1, MZF1, MZT1, MZT2A, MZT2B, NCAPD2, NCAPD3, NCAPG, NCAPG2, NCAPH, NCAPH2, RGCC, SLAMFI, SLAMF6, SLAMF7, SLAMF8, SLAMF9, TOGARAM1, TOGARAM2, UBA1, UBA2, UBA3, UBA5, UBA52, UBA6, UBA7, UBAC1, UBAC2, UBALD1, UBALD2, UBAP1, UBAPIL, UBAP2, UBAP2L, UBASH3A, UBASH3B and VCL.

In some embodiments, proteins involved in host response to viral infection comprise: FCN1, GSN, EML1, ARFGEF2, CD14, SLAMFI, FCRL3, UBASH3A, RGCC, LMNA, NCAPG, FCRL3, DAND5, CTSL, MAPK11, VCL, TOGARAM1, KIF18A, MS4A1, CD19, CD79B, MZB1, IRF8, CD1C, IL7R, CD8A, CD3D, CD3G, CD3E, CD4, GZMB, KLRB1, NCR1, FCGR3, HLA-DRB5, HLA-DRA, CD68, ITGAX, CD14, ITGAM, CFD, CD163, SOD2, LCN2, CD4177, CD45, IL-10, CCL2, CCL3, CCL4 and Ki67. The expression level of proteins involved in host response to viral infection can change during viral infection.

### Methods of Detecting and Predicting Viral Presence

Disclosed herein include methods for detecting microbes in a sample. The method can comprise: converting a plurality of reference sequences to a plurality of comma-free reference codes; converting a plurality of sample sequences to a plurality of comma-free sample codes; and aligning the plurality of comma-free reference codes to the plurality of comma-free sample codes to generate a microbe profile of the sample, thereby detecting the presence of one or more microbes in the sample.

Disclosed herein include methods for predicting or detecting microbes in a sample. In some embodiments, the method comprises: providing a model with a training dataset to determine a weight of each gene in the training data, wherein the model is a logistic regression modal, and wherein the training dataset comprises sequencing data of one or more cells; determining one or more signature genes, wherein the signature genes have weights no less than a threshold; providing a trained model with a testing dataset, wherein the trained model is parameterized with the weight of the signature genes and wherein the testing dataset comprises sequencing data of one or more cells in the sample; and determining a probability of presence of the microbes using the trained model, thereby determining the presence or absence of the microbes in the sample.

### Sample and Sample Sequences

In some embodiments, the sample comprises cells that are infected or suspected to be infected with microbes (e.g., viruses or bacteria). The cells can be plant cells, animal cells, bacterial cells, paleobacterial cells, fungal cells, mammalian cells, insect cells, avian cells, fish cells, amphibian cells, spore animal cells, human cells or non-human primate cells.

In some embodiments, the plurality of sample sequences comprise amino acid sequences and/or nucleic acid sequences. For example, the sample sequences can be DNA sequences and/or RNA sequences. In some embodiments, the sample sequences comprise sequences of the whole genome and/or transcriptome of the cells. In some embodiments, the plurality of sample sequences comprise mRNA sequences. In some embodiments, the mRNA sequences are obtained from a single cell. The nucleic acid sample sequences can be obtained using any sequencing methods, including both mass sequencing and single-cell sequencing. The mass sequencing technologies compatible with the method disclosed herein can be next generation sequencing (NGS) technologies. Multiple NGS platforms which are commercially available or which are mentioned in the literature can be used in combination of the method disclosed herein. Non-limiting examples of such NGS technologies/platforms are: 1) The sequencing-by-synthesis technology known as pyrosequencing (e.g. implemented in the GS-FLX 454 Genome Sequencer™ of Roche-associated company 454 Life Sciences (Branford, Conn.)); 2) The sequencing-by-synthesis approaches developed by Solexa (now part of Illumina Inc., San Diego, Calif) which is based on reversible dye-terminators (e.g., in the Illumina/Solexa Genome Analyzer™ and in the Illumina HiSeq 2000 Genome Analyzer™; 3) Sequencing-by-ligation approaches (e.g., implemented in the SOLid™ platform of Applied Biosystems (now Life Technologies Corporation, Carlsbad. Calif.) and the Polonator™ G.007 platform of Dover Systems (Salem, N.H.)); 4) Single-molecule sequencing technologies (e.g., implemented in the PacBio RS system of Pacific Biosciences (Menlo Park, Calif.) or in the HeliScope™ platform of Helicos Biosciences (Cambridge, Mass.)), 5) Nano-technologies for single-molecule sequencing in which various nanostructures are used (e.g., the GridON™ platform of Oxford Nanopore Technologies (Oxford, UK), the hybridization-assisted nano-pore sequencing (HANS™) platforms developed by Nabsys (Providence, R.I.), and the proprietary ligase-based DNA sequencing platform with DNA nanoball (DNB) technology called combinatorial probe-anchor ligation (cPAL™)); 6) Electron microscopy based technologies for single-molecule sequencing (e.g., those developed by LightSpeed Genomics (Sunnyvale, Calif.) and Halcyon Molecular (Redwood City, Calif.)); and 7) Ion semiconductor sequencing which is based on the detection of hydrogen ions that are released during the polymerisation of DNA (e.g., Ion Torrent Systems (San Francisco, Calif.).

Single-cell nucleic acid sequencing technologies and methods using NGS and Next Next Generation Sequencing (NNGS) (e.g., nanopores) are also commercially available. These single-cell technologies typically incorporate markers or barcodes for each cell and molecule, reverse transcription for RNA sequencing, amplification and pooling of sample for NGS and NNGS library preparation and analysis. The single-cell sequencing technologies used in combination with the method disclosed herein allows tracking of cell from which the nucleic acids derived from and counting of the number of nucleic acids sequences. The racking of cell from which the nucleic acids derived from can be achieved by the incorporation of cell barcodes. In some embodiments, the cell barcodes associated with the same cell are the same, and wherein the cell barcodes associated with different cells are different. The counting of the number of nucleic acids sequences can be achieved by the use of unique molecular identifiers (UMIs). In some embodiments, the UMIs associated with the same cell are different. In some embodiments, each of the plurality of sample sequences comprises a cell barcode and/or a UMI.

Mutation can occur frequently in viral sequences. Since alignment-based viral identification methods rely on the similarity/identity between sample sequences and reference sequences, mutations can impact the accuracy of alignment-based viral identification methods. Thus, the ability of identifying viral sequences with relatively high mutation rate (e.g., up to 12%) can be an advantage. In some embodiments, the plurality of sample sequences tested using the methods disclosed herein comprise at least one mutation. In some embodiments, the mutation is an insertion, a deletion and/or a substitution of at least one nucleotide or an amino acid. In some embodiments, the mutation is a point mutation and/or a silent mutation. In some embodiments, the mutation rate of the plurality of sample sequences is no greater than 20% (e.g., 5%, 6%, 7%, 8%, 9%, 10%, 11%, 12%, 13%, 14%, 15%, 16%, 17%, 18%, 19% or 20%). In some embodiments, the mutation rate of the plurality of sample sequences is no greater than 12%.

### Reference Sequences

In some embodiments, the plurality of reference sequences comprise amino acid sequences and/or nucleic acid sequences. For example, the reference sequences can be DNA sequences and/or RNA sequences. In some embodiments, the reference sequences comprise sequences of the whole genome and/or transcriptome of the reference species (e.g., viruses with known genome). In some embodiments, the reference sequences comprise “hallmark” sequences described herein. In some embodiments, the plurality of reference sequences comprise amino acid and/or nucleic acid sequences conservative in virus (e.g., RdRp or nucleic acid sequences encoding RdRp). In some embodiments, the reference sequences comprise amino acid sequences of and/or nucleic acid sequences encoding RdRp and/or RdDp. In some embodiments, the reference sequences can comprise sequences of 16S rRNA. In some embodiments, the reference sequences can comprise non-microbial sequences (e.g., antimicrobial amino acid sequences or nucleic acid sequences encoding antimicrobial peptides).

In some embodiments, the reference sequences allows the determination of taxonomy source of each reference sequence. In some embodiments, the reference sequences are clustered into species-like operational taxonomic units (sOTUs). In some embodiments, the sOTUs comprises taxonomy source of each of the plurality of references sequences. In some embodiments, the reference sequences comprise sequences from at least 6,000 species (e.g., 6,000 species, 7,000 species, 8,000 species, 9,000 species, 10,000 species, 11,000 species, 12,000 species, 13,000 species, 14,000 species, 15,000 species, 20,000 species, 25,000 species, 30,000 species, 35,000 species, 40,000 species, 45,000 species, 50,000 species, 60,000 species, 70,000 species, 80,000 species, 90,000 species, 100,000 species, 110,000 species, 120,000 species, 130,000 species, 140,000 species, 150,000 species, 160,000 species, 170,000 species, 180,000 species, 190,000 species, 200,000 species, 300,000 species, 400,000 species, 500,000 species, 600,000 species, 700,000 species, 800,000 species, 900,000 species or 1,000,000 species).

After converting the reference sequences to comma-free reference codes, multiple reference sequences can correspond to the same comma-free reference code. Without being bounded by any theory, this may be due to the occurrence of the ambiguous amino acid characters depending on the conversion methods used. Therefore, the methods disclosed herein can further comprise removing duplicate comma-free reference codes.

### Conversion to Comma-Free Code

In some embodiments, the sample sequences and/or the reference sequences are converted to a “shared” language. The “shared” language can be a code having only one way of correct reading, as described herein. For example, the “shared” language can be a genetic code, such as comma-free code or circular codes. In some embodiments, the length of the comma-free codes is 10-3000 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 60 nucleotides, 70 nucleotides, 80 nucleotides, 90 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1,000 nucleotides, 2,000 nucleotides or 3,000 nucleotides). In some embodiments, the length of the comma-free codes is 31 nucleotides.

In some embodiments, the length of the comma-free reference codes is 10-3000 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 60 nucleotides, 70 nucleotides, 80 nucleotides, 90 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1,000 nucleotides, 2,000 nucleotides or 3,000 nucleotides). In some embodiments, the length of the comma-free reference codes is 31 nucleotides.

In some embodiments, the length of the comma-free sample codes is 10-3000 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 60 nucleotides, 70 nucleotides, 80 nucleotides, 90 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1,000 nucleotides, 2,000 nucleotides or 3,000 nucleotides). In some embodiments, the length of the comma-free sample codes is 31 nucleotides.

In some embodiments, a sample sequence corresponds to one comma-free sample code. In some embodiments, a sample sequence corresponds to multiple comma-free sample codes. In some embodiments, all or some of the multiple comma-free sample codes are used for translated alignment disclosed herein. In some embodiments, one of the multiple comma-free sample codes is used for translated alignment disclosed herein. Therefore, the method disclosed herein can further comprise selecting the comma-free sample code having the highest similarity to the comma-free reference codes for subsequence analysis.

The conversion of sequences (e.g., sample sequences and/or reference sequences) to comma-free codes (e.g., comma-free sample codes and/or comma-free reference codes) keeps the taxonomy source information of the sequences in the comma-free codes. In some embodiments, each of the plurality of comma-free reference sequences comprises taxonomy source information of its corresponding reference sequence.

In some embodiments, converting the plurality of reference sequences to the plurality of comma-free reference codes comprises converting each reading frame to a comma-free code, and/or wherein converting the plurality of sample sequences to the plurality of comma-free sample codes comprises converting each reading frame to a comma-free code.

### Host Sequence Masking

The methods disclosed herein can identify or predict viral presence in a host cell using sequencing data obtained from the host cell. The majority of sequencing reads obtained from the cells are host cell reads, which belongs to the genome or transcriptome of the host instead of the viruses to be detected. Therefore, it is advantageous to remove the host reads for several reasons. The presence of host reading during alignment can result in misclassification of host reads as viral reads. Moreover, the large amount of host reads can slow down the operation of the systems or platforms disclosed herein. Therefore, the method disclosed herein can further comprise removing host sequences or host reads from the sample sequences.

Removal of host sequences can be achieved by different methods with different degrees of stringency/conservativeness. The reference sequences (e.g., viral sequences) may contain sequences shared with the host sequences. In some embodiments, the host sequences comprise genomic sequences and/or transcriptomic sequences. Thus, it is possible that some sequencing reading are or comprise such shared sequences. Different host masking methods classifies these shared sequence in different manner. For example, the sequencing reads can be aligned to host sequences before translated alignment. This masking method removes any sequencing reads that have some alignment with the host sequences. The alignment can be with the host genome and/or host transcriptome. The sequencing reads removed by this making method can comprise: 1) reads aligned to only shared sequence, 2) reads aligned to host-specific sequences, 3) reads aligned to sequences spanning the shared sequences and host-specific sequences, and 4) reads aligned to sequences spanning the shared sequences and reference-specific sequences (e.g., virus-specific sequences). The alignment to host sequences and removal of host reads can be conducted before the conversion of sample sequences to comma-free sample codes. In some embodiments, removing sample sequences of the plurality of sample sequences originated from host comprises removing sample sequences of the plurality of sample sequences aligned to host sequences to obtain a plurality of pre-aligned sample sequences. In some embodiments, converting the plurality of sample sequences to the plurality of comma-free sample codes comprises converting the plurality of pre-aligned sample sequences to the plurality of comma-free sample codes.

In some embodiments the removal of host reads is conducted after conversion of sample sequences to comma-free sample codes. To align with the comma-free sample codes, the host sequences can also be converted to comma-free codes. Therefore, the method disclosed herein can further comprise: converting host sequences to a plurality of comma-free host codes; and aligning the plurality of comma-free sample codes to the comma-free host codes. In some embodiments, converting the host sequences to the plurality of comma-free host codes comprises converting each reading frame of the host sequences to comma-free codes.

Removal of host reads can be conducted using a distinguishing list (D-list). The D-list can comprise amino acid sequences, nucleic acid sequences and/or comma-free codes. The D-list can comprises shared sequences and/or host-specific sequences. In some embodiments, the removal of host reads can comprise remove reads aligned to sequences on the D-list.

The method can further comprise removing comma-free sample codes of the plurality of comma-free sample codes that comprise a portion aligned to the host specific sequence. The method can further comprise removing comma-free sample codes of the plurality of comma-free sample codes that lack a reference specific sequence, wherein the reference specific sequence aligns to the plurality of comma-free reference codes but not the comma-free reference codes comma-free host codes.

### Translated Alignment and Microbe Profile Analysis

The translated alignment methods disclosed herein can comprise aligning the plurality of comma-free reference codes to the plurality of comma-free sample codes In some embodiments, aligning the plurality of comma-free reference codes to the plurality of comma-free sample codes comprises determining similarity between the plurality of comma-free reference codes and the plurality of comma-free sample codes. In some embodiments, aligning the plurality of comma-free reference codes to the plurality of comma-free sample codes comprises selecting the comma-free sample codes of the plurality of comma-free sample codes having at least 50% (e.g., 50%, 60%, 70%, 80%, 90% or 100%) similarity to the comma-free reference codes of the plurality of comma-free reference codes for subsequent analysis. In some embodiments, sample sequences corresponding to comma-free sample codes having at least 50% (e.g., 50%, 60%, 70%, 80%, 90% or 100%) similarity to the comma-free reference codes are classified as viral sequences or microbe sequences. In some embodiments, the sample sequences can be ranked according to the similarity between their corresponding comma-free sample codes to the comma-free reference codes. The sample sequences whose corresponding comma-free sample codes have higher similarity to the comma-free reference codes are ranked on the top. In some embodiments, the top ranked (e.g., top 200 ranked or top 50% ranked) sample sequences are classified as viral sequences or microbe sequences and/or are selected for subsequent analysis.

In some embodiments, the microbe profile comprises taxonomy of the microbes. Determining the taxonomy of the microbes can comprise determining the species of the microbes, determining the classification group (e.g., phylum, class, order, family or genus) of the microbes, or assigning the microbe to sOTUs. In some embodiments, the microbe profile comprises the number of total microbes. In some embodiments, the microbe profile comprises the number of microbes in each sOTUs, in each classification group (e.g., phylum, class, order, family or genus) or of each species. In some embodiments, the microbe profile comprises the number of microbes in each host cell. In some embodiments, the microbe profile comprises the number of microbes in each sOTUs, in each classification group (e.g., phylum, class, order, family or genus) or of each species in each host cell. In some embodiments, the microbe profile comprises the tropism of the microbes. The tropism of microbes comprises the tendency of the microbes to infect particular cell types.

The method can further comprise determining profile of the cells. In some embodiments, the profile of the cells comprises transcriptome profile. The cells can be host cells infected by viruses. The host cell can be plant cells, animal cells, bacterial cells, paleobacterial cells, fungal cells, mammalian cells, insect cells, avian cells, fish cells, amphibian cells, spore animal cells, human cells or non-human primate cells. In some embodiments, the profile of the cells comprises expression level of genes known to be associated with microbe infection. In some embodiments, the genes known to be associated with microbe infection are selected from MS4A1, CD19, CD79B, MZB1, IRF8, CD1C, IL7R, CD8A, CD3D, CD3G, CD3E, CD4, GZMB, KLRB1, NCR1, FCGR3, HLA-DRB5, HLA-DRA, CD68, ITGAX, CD14, ITGAM, CFD, CD163, SOD2, LCN2, CD4177, CD45, IL-10, CCL2, CCL3, CCL4 and Ki67. In some embodiments, the genes encodes effectors involved in viral infection and/or innate immune responses. In some embodiments, the genes encodes proteins involved in host response to viral infection as described herein. The method can further comprise determining the percentage of cells infected with the microbe. In some embodiments, the profile of the cells comprises type of cells infected with the microbe and abundance of each type of cells infected with the microbe.

The method can further comprise determining the stage of microbe infection. In some embodiments, the method disclosed herein detects more microbes compared to a method aligning the plurality of sample sequences to NCBI reference sequences. In some embodiments, the method disclosed herein detects at least 30% (30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 3 times, 4 times, 5 times, 6 times, 7 times, 8 times, 9 times, 10 times) more microbes compared to a method aligning the plurality of sample sequences to NCBI reference sequences. In some embodiments, the method disclosed herein detects at least 30% (30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 3 times, 4 times, 5 times, 6 times, 7 times, 8 times, 9 times, 10 times) more viral species compared to a method aligning the plurality of sample sequences to NCBI reference sequences. In some embodiments, the method detects microbes without a sequence included in the NCBI database. In some embodiments, the method detects microbes without a sequence included in the plurality of reference sequences.

In some embodiments, the method generates microbe profile with at least 60% (e.g., 60%, 70%, 80%, 90% or 100%) accuracy. In some embodiments, the method generates microbe profile with at least 90% accuracy. In some embodiments, the method disclosed herein detects and/or predicts the presence or absence of microbes (e.g., viruses) of at least 8,000 species (e.g., 8,000 species, 9,000 species, 10,000 species, 11,000 species, 12,000 species, 13,000 species, 14,000 species, 15,000 species, 20,000 species, 25,000 species, 30,000 species, 35,000 species, 40,000 species, 45,000 species, 50,000 species, 60,000 species, 70,000 species, 80,000 species, 90,000 species, 100,000 species, 110,000 species, 120,000 species, 130,000 species, 140,000 species, 150,000 species, 160,000 species, 170,000 species, 180,000 species, 190,000 species, 200,000 species, 300,000 species, 400,000 species, 500,000 species, 600,000 species, 700,000 species, 800,000 species, 900,000 species or 1,000,000 species).

### Host Gene Expression and Microbe Profile Analysis

The methods disclosed herein can comprise predicting or detecting microbe presence in a sample. In some embodiments, the method comprises training a model using a training dataset. In some embodiments, the model is a logistic regression modal.

In some embodiments, the training dataset comprises sequencing data. The sequencing data can comprise amino acid sequences and/or nucleic acid sequences. The sequencing data can comprise DNA sequences and/or RNA sequences (e.g., mRNA sequences). In some embodiments, the sequencing data comprises genome and/or transcriptome of one or more cells. In some embodiments, the training dataset comprises count of sequences (e.g., genes). For example, the training dataset can comprise count of mRNA of all genes or selected genes in the one or more cells. In some embodiments, the selected genes comprises highly variable genes in the one or more cells. The highly variable genes are genes with expression level change meeting certain criteria in response to stimulus. In the context of viral presence identification, the highly variable genes are those with expression level change during viral infection. The highly variable genes can be different during infection of different viruses and at different stages of viral infection. Methods of determining highly variable genes are described in Tim Stuart, Andrew Butler, Paul Hoffman, Christoph Hafemeister, Efthymia Papalexi, William M. Mauck, Yuhan Hao, Marlon Stoeckius, Peter Smibert, and Rahul Satija. Comprehensive integration of single-cell data. Cell, 177(7):1888-1902; Zheng, Grace X Y, et al. “Massively parallel digital transcriptional profiling of single cells.” Nature communications 8.1 (2017): 14049; Rahul Satija, Jeffrey A Farrell, David Gennert, Alexander F Schier, and Aviv Regev. Spatial reconstruction of single-cell gene expression data. Nature Biotechnology, 33(5):495-502. The highly variable genes can be ranked by their variance of expression. In some embodiments, the selected genes comprises the top (e.g., top 50, top 100, top 150, top 200, top 250, top 300, top 350, top 400, top 450, top 500, top 550, top 600, top 650, top 700, top 750, top 800, top 850, top 900, top 950, top 1000) high variable genes. In some embodiments, the training dataset comprises cell type of each cell of the one or more cells. In some embodiments, the training dataset comprises infection status of each cell of the one or more cells. In some embodiments, infection status comprises the presence or absence of microbes, taxonomy of the microbes, and stage of infection.

Using the training dataset, the model can determine a weight for each genes. Weights can be determined for all genes in a cell. The weights are used to parameterize the model. In some embodiments, the model is parameterized with weights of all genes. In some embodiments, the model is parameterized with weights of highly variable genes. In some embodiments, the model is parameterized with weights of signature genes. The signature genes can have weights no less than a threshold. In some embodiments, the threshold is 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.25, 0.3, 0.35, 0.4, 0.45 or 0.5. In some embodiments, the signature genes are genes encoding: proteins regulating cytokine production, proteins regulating viral entry into host cell, proteins regulating viral life cycle, and/or receptors mediating endocytosis. The signature genes include, e.g., genes encoding proteins FCN1, GSN, EML1, ARFGEF2, CD14, SLAMFI, FCRL3, UBASH3A, RGCC, LMNA, NCAPG, FCRL3, DAND5, CTSL, MAPK11, VCL, TOGARAM1 or KIF18A.

To determining viral presence in the sample, the model parameterized with weights of genes can be fed with testing data that comprises sequencing data of the sample. The sequencing data can comprise amino acid sequences and/or nucleic acid sequences. The sequencing data can comprise DNA sequences and/or RNA sequences (e.g., mRNA sequences). In some embodiments, the sequencing data comprises genome and/or transcriptome of one or more cells in the sample. In some embodiments, the testing dataset comprises count of sequences (e.g., genes). For example, the testing dataset can comprise count of mRNA of all genes or selected genes in the one or more cells. The selected genes can be the signature genes identified using the methods disclosed herein. In some embodiments, the testing dataset comprises cell type of each cell of the one or more cells in the sample.

In some embodiments, the model parameterized with the weights of genes calculates the probability of presence of the microbes based on the testing dataset, thereby determining the presence or absence of the microbes in the sample. In some embodiments, determining the presence or absence of microbes in the sample comprises determining whether the presence or absence of microbes in each of the one or more cells in the sample. In some embodiments, the microbe is determined as present in the sample, if the probability of presence of the microbes is at least 50% (e.g., 50%, 60%, 70%, 80%, 90% or 100%). In some embodiments, determining the presence or absence of microbes in the sample comprises determining taxonomy of the microbes. In some embodiments, determining the presence or absence of microbes in the sample comprises determining the number of microbes. In some embodiments, determining the presence or absence of microbes in the sample comprises determining the number of each microbe species in each cell of the one or more cells in the sample. In some embodiments, the method generates microbe profile with at least 60% (e.g., 60%, 70%, 80%, 90% or 100%) accuracy. In some embodiments, the method generates microbe profile with at least 90% accuracy.

### Systems and Platforms

Disclosed herein includes systems and platforms for performing the methods for predicting or detecting microbes in a sample disclosed herein through translated alignment. In some embodiments, the systems and platforms comprises means for converting a plurality of reference sequences to a plurality of comma-free reference codes. In some embodiments, the systems and platforms comprises means for converting a plurality of sample sequences to a plurality of comma-free sample codes. In some embodiments, the systems and platforms comprises means for aligning the plurality of comma-free reference codes to the plurality of comma-free sample codes to generate a microbe profile of the sample, thereby detecting the presence of one or more microbes in the sample.

Disclosed herein includes systems and platforms for performing the methods of predicting or detecting microbes in a sample disclosed herein through host gene expression. In some embodiments, the systems and platforms comprises means for training a model with a training dataset to determine a weight of each gene in the training data. In some embodiments, the model is a logistic regression modal. In some embodiments, the training dataset comprises sequencing data of one or more cells.

In some embodiments, the methods disclosed herein comprises determining one or more signature genes. In some embodiments, the signature genes have weights no less than a threshold. In some embodiments, the systems and platforms comprises means for parameterizing the model with the weight of the signature genes to obtain a trained model. In some embodiments, the testing dataset comprises sequencing data of one or more cells in the sample. In some embodiments, the systems and platforms comprises means for determining a probability of presence of the microbes using the trained model, thereby determining the presence or absence of the microbes in the sample.

### EXAMPLES

Some aspects of the embodiments discussed above are disclosed in further detail in the following examples, which are not in any way intended to limit the scope of the present disclosure.

### Materials and Methods

The following experimental materials and methods were used for Example 1 described below.

### 1. Developing Kallisto Translated Search and Optimization for the Identification of Viral RNA

### Building Kallisto Translated Search and Choosing a New “Genetic Code”

To perform translated alignment, the nucleotide and amino acid sequences were translated into a shared “language,” by translating nucleotide sequences to amino acid sequences or vice versa. Since kallisto encoded each nucleotide in 2 bits, allowing a total of 4 distinct nucleotides to be encoded, encoding the 20 different amino acids translated from nucleotide sequences was not feasible. Moreover, reverse translating the amino acid sequences to nucleotide sequences would be intractable due to the redundancy in the genetic code. Therefore, the nucleotide sequences were translated and the amino acid sequences were reverse translated using a fixed synthetic code designed to reduce spurious alignments. Two different sets of codes were explored for this translation: 1) a comma-free code and 2) a code that maximized the Hamming distance between frequently occurring amino acids. The Hamming distances obtained from the two sets of codes are shown in FIG. 18A. While maximizing Hamming distance was advantageous in terms of avoiding sequence multimapping, a comma-free code prevented off-frame alignment since any k-mers formed by adjacent words would not be included in the dictionary. As shown in FIG. 18B, the comma-free code recalled viral sequences notably better than maximizing the Hamming distance between amino acids, likely due to the reduction of ambiguity introduced by frame shifts.

### Optimization of PalmDB for the Identification of Viral Reads in RNA Sequencing Data

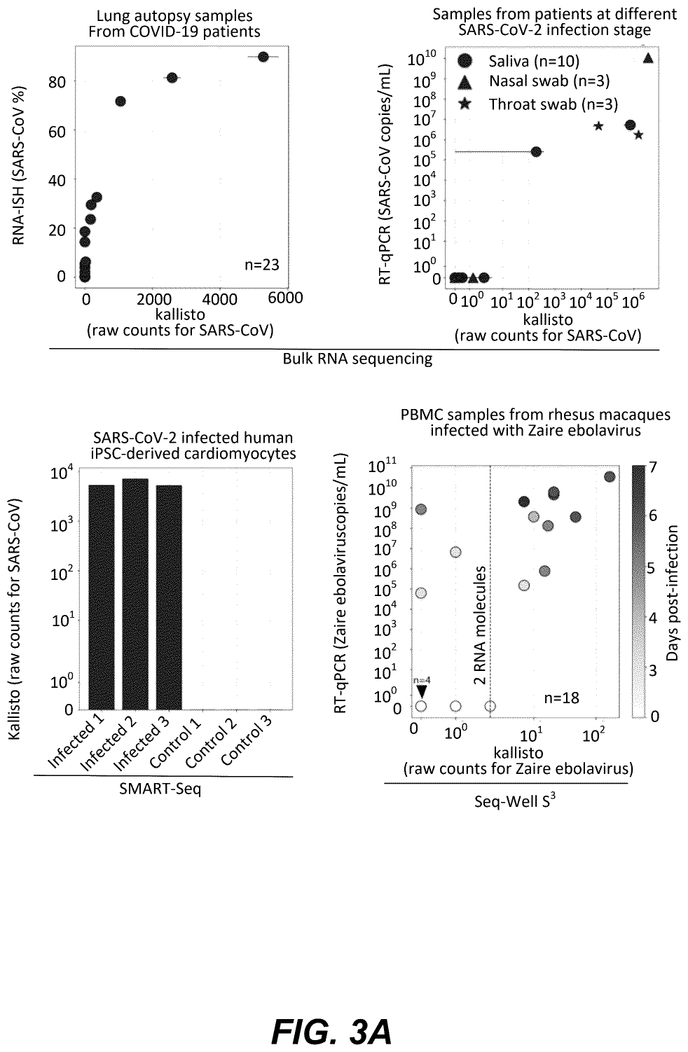

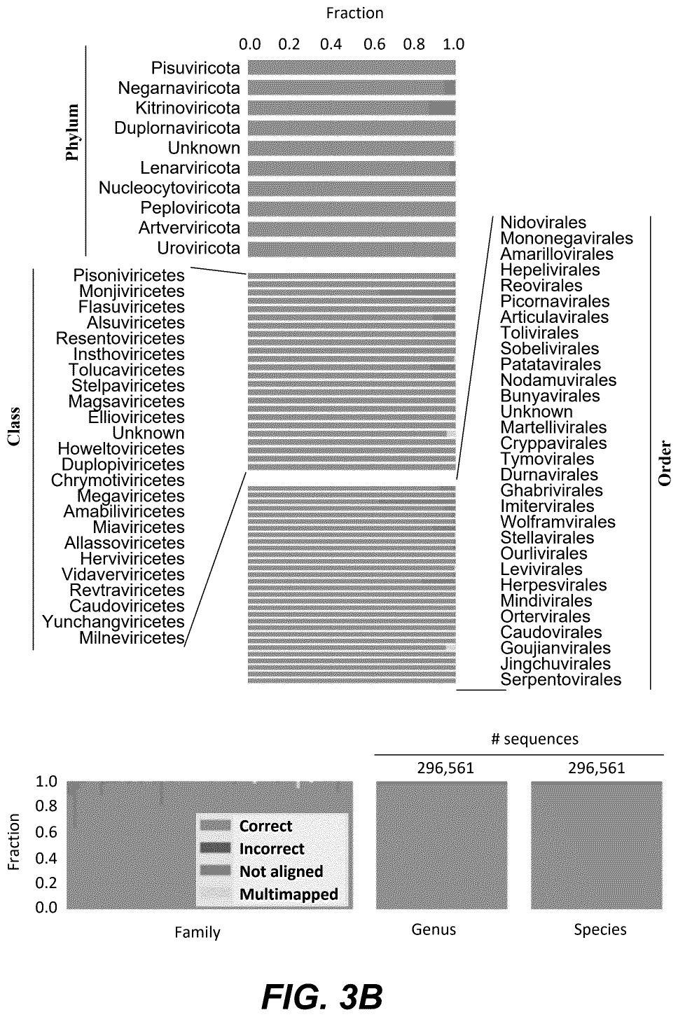

Due to the occurrence of the ambiguous amino acid characters (e.g., B, J and Z), 62 out of 296,623 viral sequences were transformed into identical sequences after reverse translation to comma-free code. The identical sequences were merged and assigned a representative virus ID. Due to the high similarity between viral RdRP sequences, the loss of aligned sequences due to multimapping to several reference sequences was a major concern. Moreover, the necessity of reverse translating the amino acid sequences further decreases the Hamming distance between reference sequences by approximately 30% (FIG. 18D). To overcome this problem, the amino acid sequences were clustered based on 99% similarity using the algorithms (e.g., MMseqs or MMseqs2). This resulted in 6,518 clusters with high concordance of taxonomy labels between sequences in the same cluster (FIG. 18E). However, although clusters were computed correctly based on their concordance with taxonomy, this resulted in 67.4% of sOTUs not being detected anymore, while only 3.3% undetected when using the complete index. To solve this issue, the sOTUs were grouped instead, treating virus IDs with the same taxonomy across all main taxonomic ranks like transcripts of the same gene. The database is available at tinyurl.com/4wd33rey. This retained the alignment percentage of the complete index while allowing highly accurate taxonomic assignment and minimal sequence loss to multimapping (FIG. 3B). It is noteworthy that the default kallisto k-mer length was set to be 31 nucleotides, which equals only 10 amino acids. Given the architecture of the kallisto version (e.g., 0.50.0 or 0.50.1), which was optimized for 64-bit k-mers with each nucleotide occupying two bits, k cannot be set >31. This might change in future versions.

### 2. Validation and Benchmarks

### Visualization of the Kraken2 and Kallisto Translated Search Alignments of ZEBOV Sequences

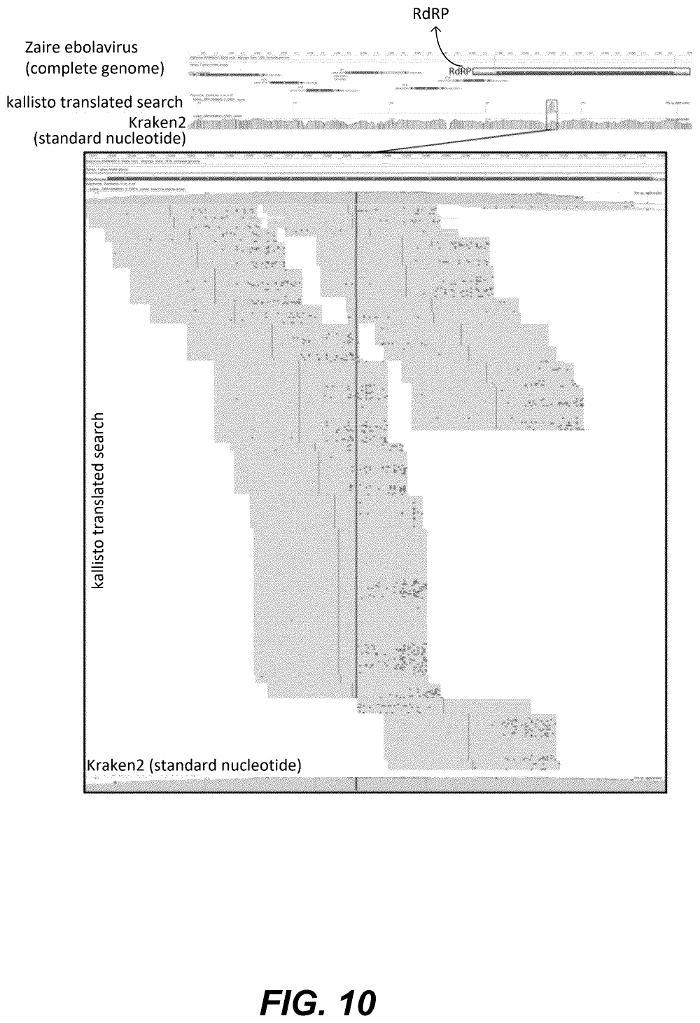

Kotliar et al. performed single-cell RNA sequencing of PBMC samples from rhesus macaques after infection with ZEBOV. A subset of the data obtained by Kotliar et al. at 8 days post-infection with ZEBOV was used to visualize the identification of RdRP sequences using kallisto (e.g., v0.50.0 or v0.50.1) translated search. The first 100,000,000 raw sequencing reads from the GSE158390 library SRR12698539 (www.ncbi.nlm.nih.gov/geo/query/acc.cgi?acc=GSE158390) were aligned to the ZEBOV reference genome (NC_002549.1) using Kraken2 v2.1.2 and to the optimized PalmDB using kallisto translated search. Aligned reads from both workflows were extracted and realigned to the ZEBOV genome using bowtie2 v2.2.5 and SAMtools v1.6. The visualization shown in FIG. 10 was generated from the resulting sorted bam files with the NCBI Genome Workbench.

### Testing Robustness to Mutation

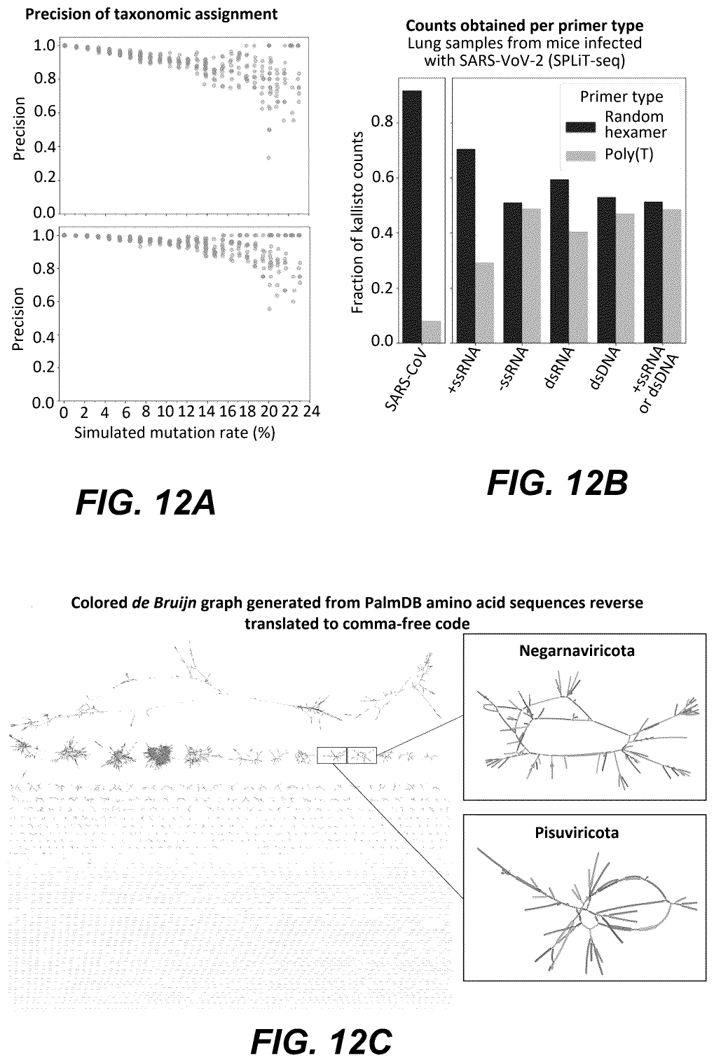

676 Zaire ebolavirus (ZEBOV) RdRP sequences were identified by aligning the first 100,000,000 raw sequencing reads from the GSE158390 library SRR12698539 to the optimized PalmDB using kallisto translated search. Mutation-Simulator (e.g., v3.0.1) was used to add random single nucleotide base substitutions to the RdRP sequences at increasing mutation rates. 10 rounds of simulated mutations per mutation rate were performed. The sequences were subsequently aligned using kallisto translated search against the complete PalmDB, Kraken2 translated search against the RdRP amino acid sequence of ZEBOV with a manually adjusted NCBI Taxonomy ID to allow compatibility with Kraken2, and kallisto standard workflow against the complete ZEBOV nucleotide genome (GCA_000848505.1). The recall percentage over all 676 sequences was subsequently calculated. For kallisto translated search, the recall percentage was calculated based on genus-level taxonomic assignment. Since the other two methods were only given the target virus sequence as a reference and did not have to distinguish between different viruses, their recall percentage was calculated based on all aligned sequences. The recall percentage over all 676 sequences for the 10 rounds at each mutation rate is shown in FIG. 2C. FIG. 12A shows the precision, with which kallisto translated search identified the correct virus versus other taxonomies at each mutation rate. The recall and precision at mutation rates >0 were fitted with an inverse sigmoid function using non-linear least squares using the scipy.optimize.curve_fit function (scipy v1.11.1).

### Alignment and Quantification of Viral Counts in Validation Datasets

The sequencing reads for each library used in the validation (FIG. 3A) were aligned with kallisto translated search against the PalmDB index D-listed with the corresponding host genome and transcriptome. The hosts were (i) human (GRCh38 Ensembl version 109) for GSE150316 (www.ncbi.nlm.nih.gov/geo/query/acc.cgi?acc=GSE150316), GSM4548303 (www.ncbi.nlm.nih.gov/geo/query/acc.cgi?acc=GSM4548303) and the SARS-CoV-2 saliva, nasal, and throat samples, (ii) mouse (GRCm39 Ensembl version 109) for GSM5974202 (www.ncbi.nlm.nih.gov/geo/query/acc.cgi?acc=GSM5974202), and (iii) rhesus macaque (Mmul_10 Ensembl version 109) and dog (ROS_Cfam_1.0 Ensembl version 109) for GSE158390 (www.ncbi.nlm.nih.gov/geo/query/acc.cgi?acc=GSE158390). Count matrices were generated with bustools (v0.43.1). FIG. 3A shows the total raw counts obtained for each target virus species. RT-qPCR and RNA-ISH counts were reproduced from the original publications.

### Validating the Alignment of Nucleotide Sequences to an Amino Acid Reference and Assessing the Accuracy of the Taxonomic Assignment

To validate the mapping of nucleotide sequences to an amino acid reference with kallisto translated search and assess the accuracy of the taxonomic assignment, all amino acid sequences in the PalmDB were reverse translated using the “standard” genetic code from the biopython (v1.79) Bio.Data.CodonTable module and DnaChisel (v3.2.10), with a slight modification to allow the ambiguous amino acids “X,” “B,” “J” and “Z” occurring in the PalmDB, which was later implemented in DnaChisel v3.2.11. A unique synthetic “cell barcode” was generated for each resulting nucleotide sequence. The sequences were aligned to the optimized amino acid PalmDB with kallisto translated search, keeping track of each sequence individually as if they were an individual cell. The synthetic barcodes allowed subsequent analysis of the alignment result for each individual sequence. The accuracy of the obtained taxonomy based on the virus ID to sOTU mapping provided by PalmDB is shown in FIG. 3B. For each sequence, “correct” or “incorrect” taxonomic assignments were distinguished. If the sequence did not return any results, whether it was “multimapped” or “not aligned” (the sequence was not aligned to any target), at each taxonomic rank.

### 3. Analysis of Macaque PBMC Data

Kotliar et al. performed single-cell RNA sequencing of PBMC samples from 19 rhesus macaques at different time points during Ebola virus disease (EVD) after infection with ZEBOV (EBOV/Kikwit; GenBank accession MG572235.1; Fiilovridae: Zaire ebolavirus) using Seq-Well with the S3 protocol. A subset of PBMC samples were spiked with Madin-Darby canine kidney (MDCK) cells, with genome data available in GSE158390 database. The raw sequencing data was obtained from the European Nucleotide Archive using FTP download links and ffq (v0.3.0). The data was split into 106 datasets containing 30,594,130,037 reads in total.

### Alignment to the Host Transcriptome

The rhesus macaque Mmul_10 and domestic dog ROS_Cfam_1.0 genomes were retrieved from Ensembl version 109. The reference index was built using both genomes and the kb-python (e.g., v0.28.0 with kallisto v0.50.0 or v5.50.1 and bustools v0.43.1) ref command to create a combined index containing the transcriptome of both species. The gene expression in each of the 106 datasets was quantified using the standard kallisto-bustools workflow with the “batch” and “batch-barcodes” arguments to process all files simultaneously while keeping track of each batch. The ‘x’-string “0,0,12:0,12,20:1,0,0” was used to match the Seq-Well technology. Since the Seq-Well technology does not provide a barcode on-list, a barcode on-list was generated using the “bustools allowlist” command, requiring each barcode to occur at least 1,000 times. The cell barcodes were subsequently corrected using the generated on-list and computed the count matrix using the “bustools count” function.

### Host Cell Quality Control, Filtering, and Separation of Macaque and MDCK Cells

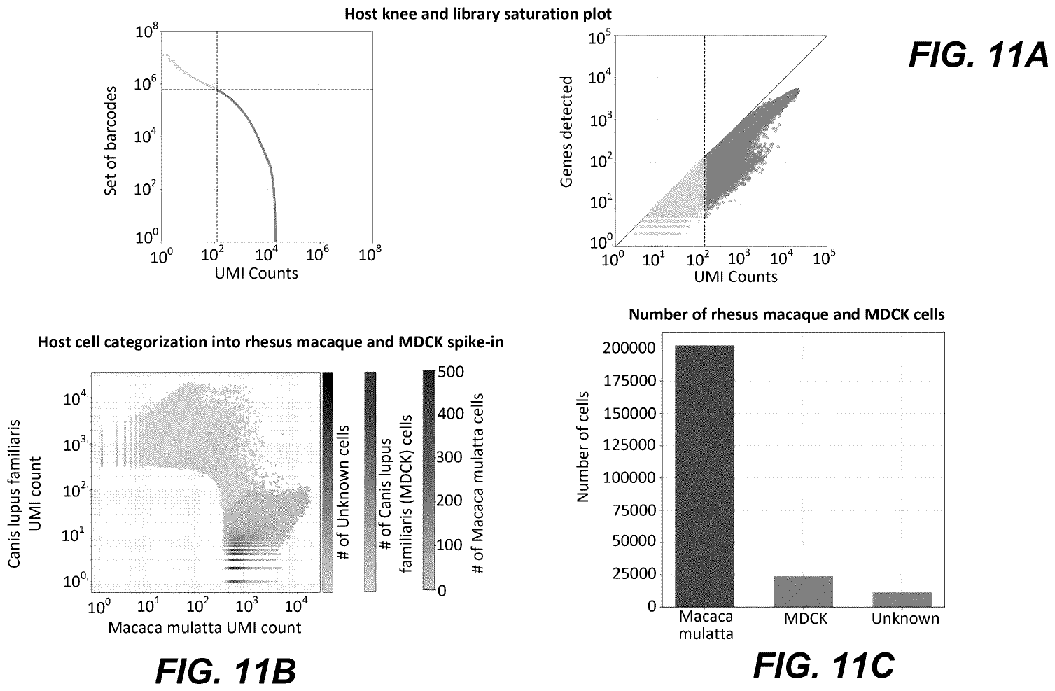

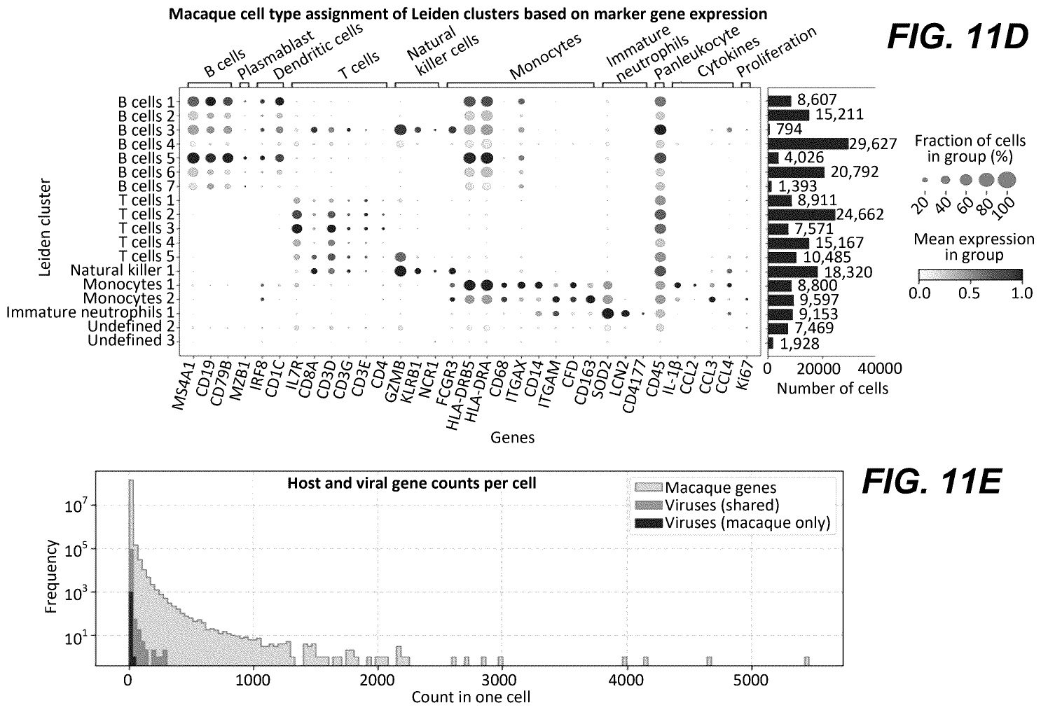

The count matrix generated by bustools was converted to h5ad using kb_python.utils.kb_utils and read into Python using anndata v0.8.0. Metadata (e.g., donor animal, the presence of an MDCK spike-in and time point) were added to the AnnData object from the SRR library metadata provided by Kotliar et al. The cell barcodes were filtered based on a minimum number of UMI counts of 125 obtained from the knee plot of sorted total UMI counts per cell (FIG. 11A), resulting in a mean UMI count of 1,401 after filtering. The cells were further filtered based on a maximum percentage of mitochondrial genes of 10%, based on a combination of macaque and dog mitochondrial genes facilitated by Scanpy (v1.9.3) and gget (v0.28.0). Cells were categorized as macaque if a maximum of 10% of their UMIs originated from dog genes and vice versa (FIG. 11B). Macaque and MDCK cells were normalized separately using log(CP10k+1) with Scanpy's normalize_total defaults of target sum 10,000 and log 1p.

### Macaque Cell Clustering and Cell Type Assignment

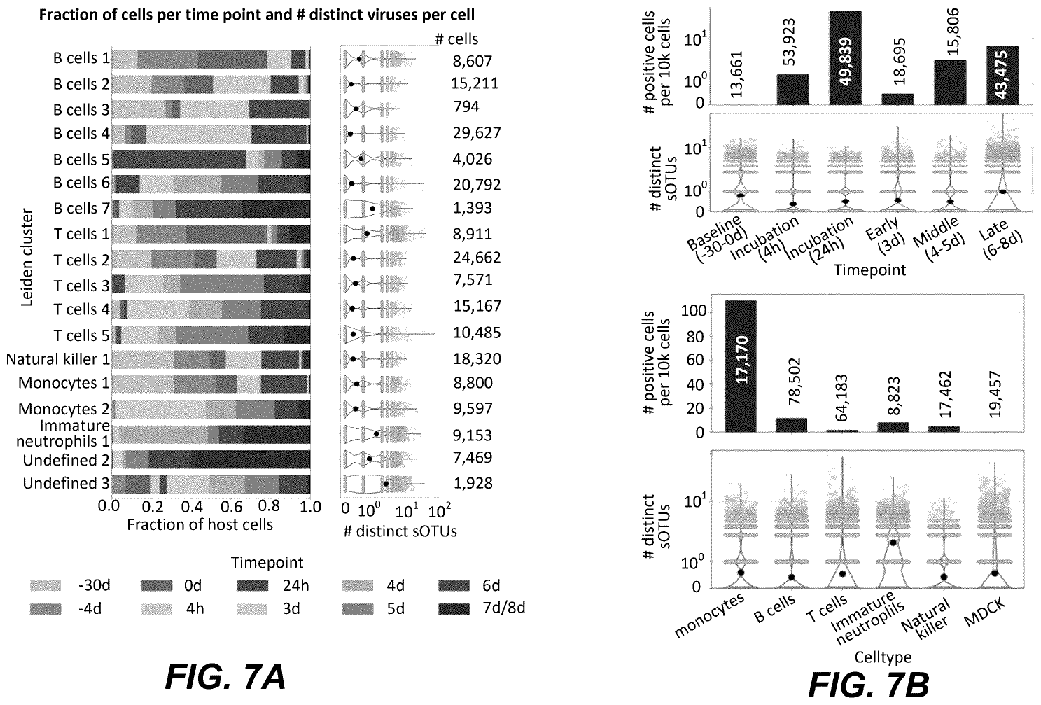

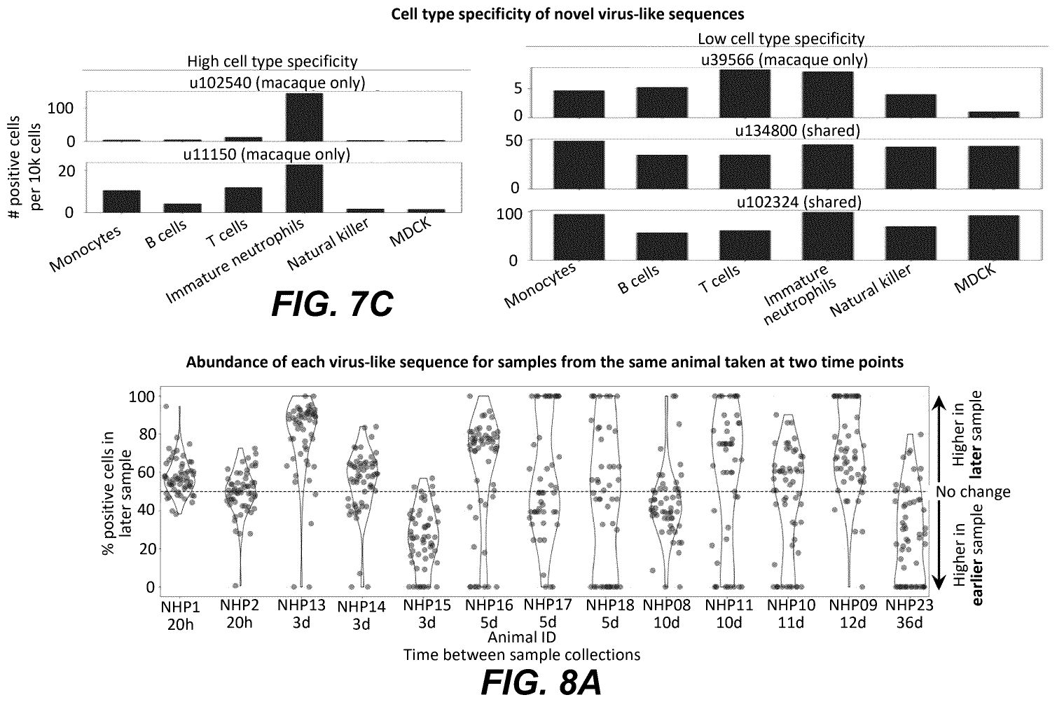

The macaque gene count matrix was transformed by PCA to 50 dimensions applied using the log-normalized counts filtered for highly variable genes using Scanpy's highly_variable_genes. Next, nearest neighbors was computed and Leiden clustering was conducted using Scanpy, resulting in 19 Leiden clusters. As shown in FIG. 7A, EVD time points were highly concordant across sequencing libraries, suggesting the lack of a batch effect. Each cluster was manually annotated with a cell type based on the expression of previously established marker genes (FIG. 11D). Cluster “Undefined 1” was omitted because it only contained 12 cells. Gene names and descriptions for Ensembl IDs without annotations were obtained using gget.

Virus Alignment with Different Masking Options

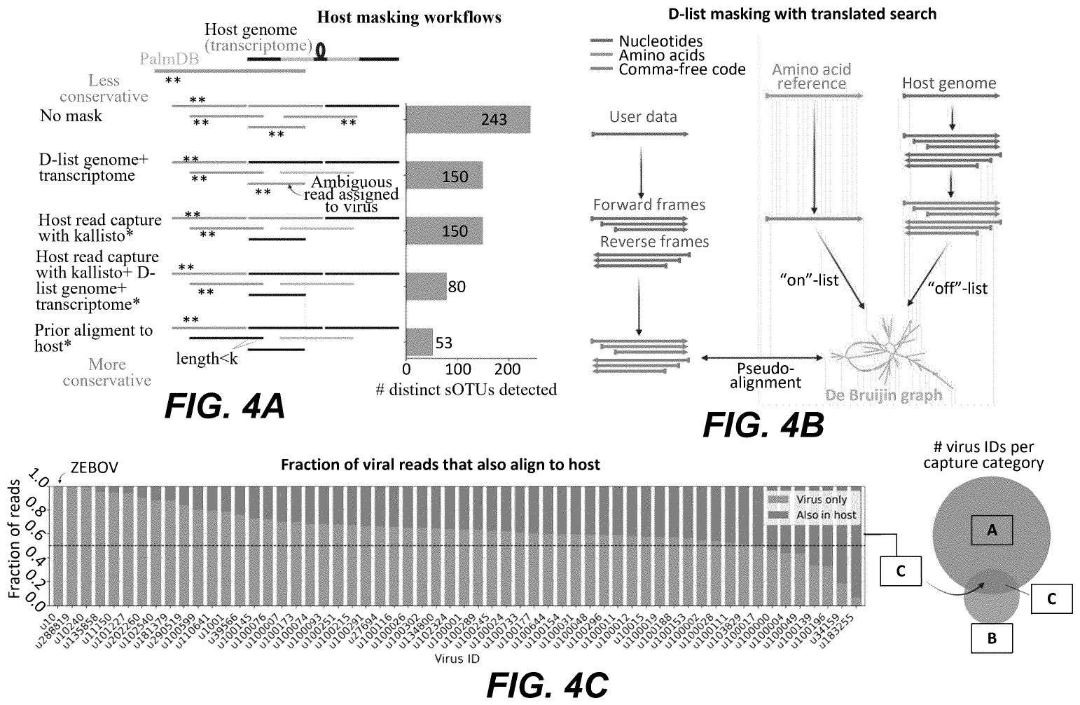

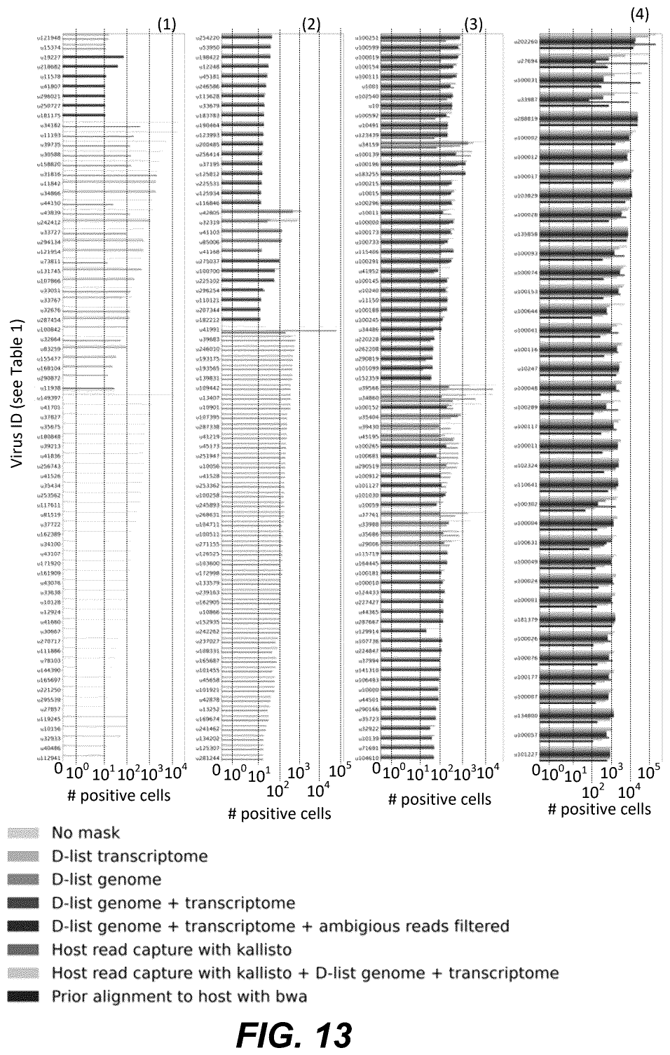

For each masking option, the gene expression was quantified in each of the 106 datasets from GSE158390 using kallisto with the “batch” and “batch-barcodes” arguments to process all files simultaneously while keeping track of each batch and with the ‘x’-string “0,0,12:0,12,20:1,0,0” to match the Seq-Well technology. kallisto translated search was initiated in the “kallisto index” and “kallisto bus” commands by adding the “-aa” flag. Following the alignment to PalmDB with any of the masking options, cell barcodes were corrected using the barcode on-list generated during the alignment to the host as described above.
(1) No mask: The raw sequencing reads were aligned to the optimized PalmDB reference files, using kallisto translated search.
(2) D-list genome+transcriptome: The raw sequencing reads were aligned to the optimized PalmDB reference using kallisto translated search with the added argument “d-list,” which was passed the concatenated macaque genome and transcriptome (Mmul_10 Ensembl version 109), and dog genome and transcriptome (ROS_Cfam_1.0 Ensembl version 109). For D-list masking options including only the genomes or transcriptomes as illustrated in FIG. 4A, only the genome or transcriptome files from both species were concatenated and passed to the ‘d-list’ argument, respectively.
(3) D-list genome+transcriptome+ambiguous reads filtered: This workflow was performed as described above for the “D-list genome+transcriptome,” except that ambiguous reads in the D-list would be thrown out as host instead of being assigned to virus (FIG. 4A). This option was explored to investigate the effect of ambiguous reads during D-list masking. However, the alignment results did not notably differ from the masking option “D-list genome+transcriptome” (FIG. 4A and FIG. 13).

TABLE 1
LIST OF VIRUS IDS IN FIG. 13

| Panel # | (1) | (2) | (3) | (4) |
|---|---|---|---|---|
| Virus IDs | u121948 | u254220 | u100251 | u202260 |
| listed from | u15374 | u53950 | u100599 | u27694 |
| top to bottom | u19227 | u198422 | u100019 | u100031 |
| | u218682 | u12248 | u100154 | u33987 |
| | u11578 | u45181 | u100111 | u288819 |
| | u41807 | u246586 | u100154 | u100002 |
| | u296021 | u113628 | u102540 | u100012 |
| | u250727 | u33679 | u10 | u100017 |
| | u181175 | u183783 | u100592 | u103829 |
| | u34182 | u190464 | u10491 | u100028 |
| | u11193 | u123993 | u123439 | u135858 |
| | u39735 | u200485 | u34159 | u100074 |
| | u30588 | u256414 | u100139 | u100153 |
| | u158820 | u37195 | u100196 | u100644 |
| | u31816 | u125812 | u183255 | u100041 |
| | u11842 | u225531 | u100215 | u100116 |
| | u34866 | u125934 | u10015 | u10247 |
| | u44150 | u116846 | u100296 | u100048 |
| | u43839 | u42805 | u10011 | u100289 |
| | u242412 | u32319 | u100000 | u100117 |
| | u33727 | u41103 | u100173 | u100011 |
| | u294134 | u85006 | u100733 | u102324 |
| | u121954 | u41168 | u115406 | u110641 |
| | u73811 | u275037 | u100291 | u100302 |
| | u131745 | u100700 | u41952 | u100004 |
| | u107866 | u225102 | u100145 | u100631 |
| | u33051 | u296254 | u10240 | u100049 |
| | u33767 | u110121 | u11150 | u100024 |
| | u32676 | u207344 | u100188 | u100001 |
| | u287454 | u182212 | u100245 | u181379 |
| | u100842 | u41991 | u34486 | u100026 |
| | u32664 | u39683 | u220228 | u100076 |
| | u83259 | u246010 | u262208 | u100177 |
| | u155477 | u193175 | u290819 | u100007 |
| | u168104 | u193565 | u101099 | u134800 |
| | u290872 | u139831 | u152359 | u100057 |
| | u11938 | u109442 | u39566 | u101227 |
| | u149397 | u13407 | u34860 | |
| | u41701 | u10901 | u100152 | |
| | u37827 | u107395 | u35404 | |
| | u35675 | u287338 | u39430 | |
| | u180848 | u41219 | u45195 | |
| | u39213 | u45173 | u100265 | |
| | u41836 | u251947 | u100681 | |
| | u256743 | u10056 | u290519 | |
| | u41526 | u41528 | u100912 | |
| | u35434 | u253362 | u101127 | |
| | u253562 | u100258 | u101030 | |
| | u117611 | u245893 | u10059 | |
| | u81519 | u268631 | u37761 | |
| | u37722 | u104711 | u33988 | |
| | u162389 | u100511 | u35686 | |
| | u34100 | u271155 | u29006 | |
| | u43107 | u126525 | u115719 | |
| | u171920 | u103600 | u164445 | |
| | u161909 | u172998 | u100181 | |
| | u43076 | u133579 | u100010 | |
| | u33638 | u239163 | u124433 | |
| | u10128 | u162905 | u227427 | |
| | u12924 | u10866 | u44365 | |
| | u41660 | u152935 | u287667 | |
| | u30667 | u242262 | u129914 | |
| | u270717 | u237027 | u107736 | |
| | u111886 | u108331 | u224847 | |
| | u78103 | u165687 | u37994 | |
| | u144390 | u101455 | u141310 | |
| | u165697 | u45658 | u106493 | |
| | u221250 | u101921 | u10000 | |
| | u295539 | u42878 | u44501 | |
| | u27857 | u13252 | u290166 | |
| | u119245 | u169674 | u35723 | |
| | u10156 | u241462 | u32922 | |
| | u40486 | u134202 | u10139 | |
| | u112941 | u125307 | u7169I | |
| | | u281244 | u104610 | |

(4) Host read capture with kallisto: The raw sequencing reads were aligned to the combined macaque and dog reference index generated during the alignment to host with “kallisto bus” with the added ‘-n’ flag. The ‘-n’ flag kept track of the read line number of each aligned read. The line numbers were added to the resulting bus file. The raw sequencing reads were also aligned to the modified PalmDB with kallisto translated search with the added ‘-n’ flag to obtain all reads that map to viral RdRPs. Subsequently, the bus file returned by kallisto translated search was split into reads that only aligned to viral RdRPs and reads that also aligned to host based on the read line numbers in the bus files. This step was performed using “bustools capture” to obtain all reads that belonged to a single batch file (of the 106 dataset files), and then, capture all reads that also aligned to host.
(5) Host read capture with kallisto+D-list genome+transcriptome: Host reads were captured with kallisto as described above under “(4) Host read capture with kallisto.” However, during the alignment of the raw sequencing reads to PalmDB with the ‘-n’ flag, the “d-list” flag was also used to mask the host genomes and transcriptomes as described above under “D-list genome+transcriptome.”
(6) Prior alignment to host with bwa: bwa v0.7.17 was installed from source. The “bwa index” command was used to generate a bwa index from the concatenated macaque and dog genomes (Mmul_10 and ROS_Cfam_1.0 from Ensembl v109). The raw sequencing reads were subsequently aligned to the bwa index using the “bwa mem” command, aligning each file separately. For each FASTQ file, the names of all unmapped reads were extracted using “samtools view” (SAMtools v1.6), and a new FASTQ file including only unmapped sequences was generated using the “seqtk subseq” command (v1.4). The resulting FASTQ files containing the sequencing reads that did not map to the host genomes were aligned to the optimized PalmDB reference files using kallisto translated search.

### Extraction and BLAST Alignment of Viral Reads

Randomly selected sequencing reads from three libraries including reads mapped to the viruses of interest were aligned to the optimized PalmDB with kallisto translated search including the ‘-n’ flag, without any host read masking. Reads that mapped to the viruses of interest were subsequently captured and extracted from the raw sequencing FASTQ files using “bustools capture” and “bustools extract.”

BLAST+v2.14.1 was installed from source and the BLAST nt database was downloaded using the update_blastdb.pl command. 10 reads were randomly chosen for each target virus for each library and were BLASTed/aligned against the nt database using the blastn algorithm. Sequences that aligned to the polyA tail were recognized by the occurrence of “AAAAAAAAAAAA” or “TTTTTTTTTTTT” in the aligned part of the subject or query sequences and removed from the results. BLAST results were subsequently plotted using pyCirclize.Circos (v1.0.0).

### Virus Quality Control

The viral count matrix generated using the “Host read capture with kallisto D-list genome transcriptome” masking workflow was converted to h5ad using kb_python.utils.kb_utils and read into Python using anndata v0.8.0. Metadata (e.g., donor animal, the presence of an MDCK spike-in and time point) were added to the AnnData object from the SRR library metadata provided by Kotliar et al. For each cell, the host species and cell type were added from the host matrices generated as described above. The virus count matrix was subsequently binarized, such that for each cell, each virus was either present or absent. The viruses were classified as “present” if the viruses were observed in ≥0.05% of cells in either species.

Virus Categorization into Shared, “Macaque Only,” and “MDCK Only” Viruses

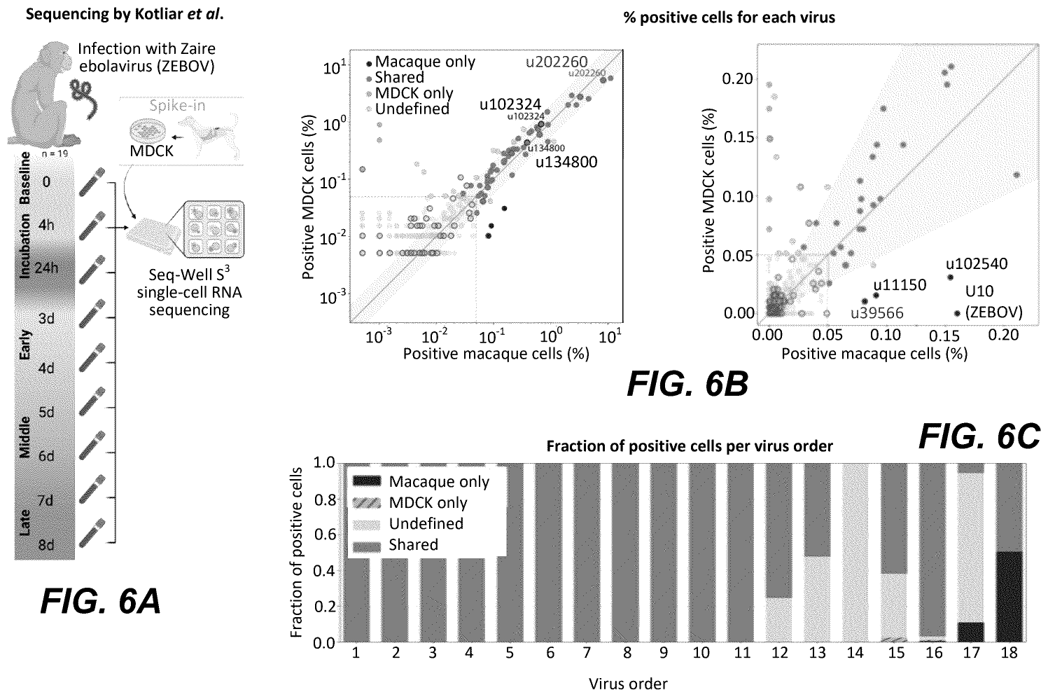

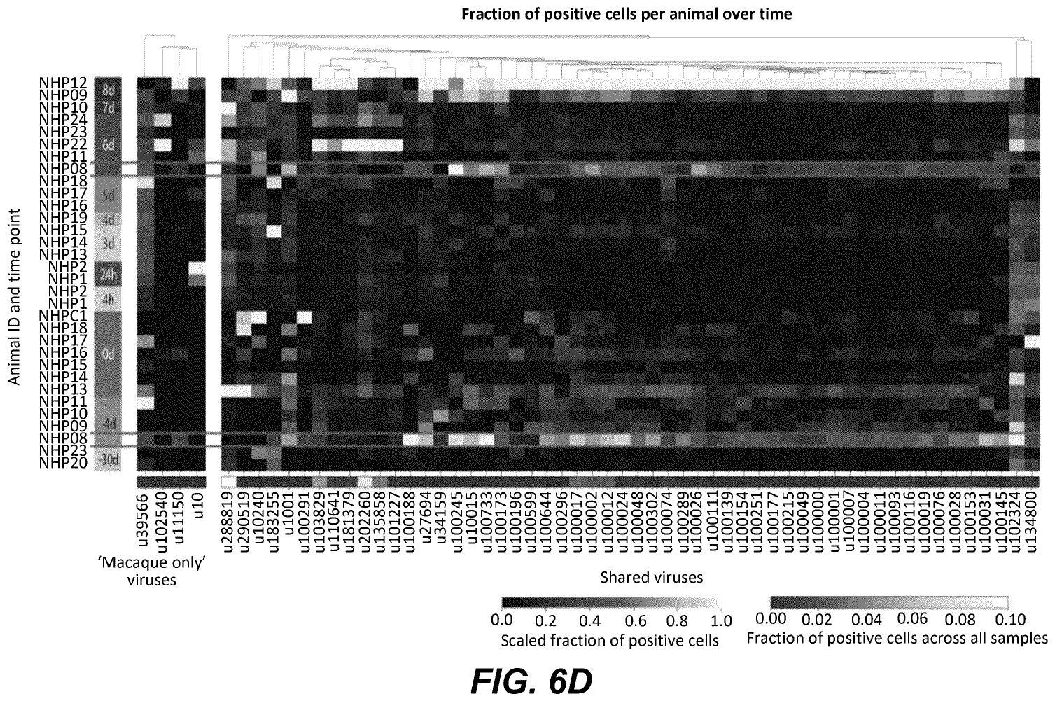

For each virus ID, the virus was defined as “shared” if the fold change between the fraction of positive macaque cells and the fraction of positive MDCK cells was less than or equal to 2. Viruses were assigned the category “macaque only” if the virus was seen in ≥0.05% of macaque cells and ≤7 MDCK cells, and vice versa for the category “MDCK only.” These thresholds were defined based on the percentages of positive cells observed for each virus in each species, as shown in FIG. 6B.

### Generation of the Krona Plot

KronaTools v2.8.1 was installed from source. A data frame containing the total numbers of positive cells for each sOTU seen in ≥0.05% of macaque cells for each animal and time point including only cells that passed host cell quality control were generated. The ktImportText tool was used to generate a Krona plot HTML file from a text file generated from this data frame.

### Logistic Regression Models to Predict Viral Presence Based on Host Gene Expression

Logistic regression modeled the log odds ratio of an event as a weighted linear combination of some predictor variables. Specifically, the natural log of the ratio of the probability p that an event occurs to the probability that it does not occur was modeled in equation (1) below.

$$ln ( \frac{p}{1 - p} ) = {\sum }_{i = 1}^{N} {β}_{i} {x}_{i} + {β}_{0} \qquad ( 1 )$$

In equation (1), each xᵢ is a predictor variable with corresponding weight βᵢ and β₀ is an intercept. Also in equation (1), p is the probability of viral presence or absence in a given cell, predicted based on a linear combination of normalized host gene count values. The normalized host gene count values are denoted as x with a total of G modeled genes. Viral presence or absence was modeled for a single virus at a time. To control for covariates, animal identifier denoted as y with a total of A animals and time point denoted as z with a total of T time points were also included, which were one-hot encoded for fits in equation (2) below.

$$ln ( \frac{p}{1 - p} ) = {β}_{0} + {\sum }_{g ∈ G} {β}_{g}^{G} {x}_{g} + {\sum }_{a ∈ A} {β}_{a}^{A} {y}_{a} + {\sum }_{t ∈ T} {β}_{t}^{T} {z}_{t} \qquad ( 2 )$$

The magnitude of the weight value for each predictor variable corresponded to that variable's influence on event probability, with large positive weights increasing the probability and large negative weights decreasing the probability of the event. Thus, an analysis of gene weights suggested which genes were likely to correlate with viral infection. For models parameterized by highly variable (HV) genes, the host (e.g., macaque) matrix was subset to highly variable genes as defined above. To reduce the occurrence of false negative viral counts, the logistic regression models were trained using the viral count matrix obtained without any masking of the host genes. However, the models were trained for viruses that were filtered based on the more conservative masking options (e.g., “macaque only” and “shared” viruses).

To further reduce the occurrence of false negative viral counts, the virus and host matrices were also filtered to include only the top 50% of cells according to the sum of raw host reads per cell before training the models. This was done to reduce the effects introduced by varying sequencing depths. For example, cells with a lower sequencing depth would have a higher likelihood of a false negative viral count. Models trained using only cells within the top 50% of sequencing depth yielded similar results, with model accuracies slightly increasing across all viruses (data not shown). Thus, it is possible the sequencing depth did not have a significant effect on model training and training using the full count matrix to maximize the number of testing and training cells would also provide models with satisfying accuracy.

For viruses with more virus-negative than virus-positive cells, half of the virus-positive cells and an equal number of virus-negative cells were randomly selected to train the logistic regression models. For viruses with more virus-positive than virus-negative cells, half of the virus-negative cells and an equal number of virus-positive cells were randomly selected for training. In both cases, the remaining cells were used for testing the performance of trained models. Given the cell-type specificity of the viruses whose presence could be predicted with high accuracy, to confirm that the models did not simply predicting cell type, virus-negative training cells were selected to be of the same cell types as virus-positive cells (FIG. 16C). The number of training and testing cells for each virus are listed in Table 2. The models were trained and tested for ZEBOV (u10) and either 5 (experiment 1) or 6 (experiment 2) novel viruses.

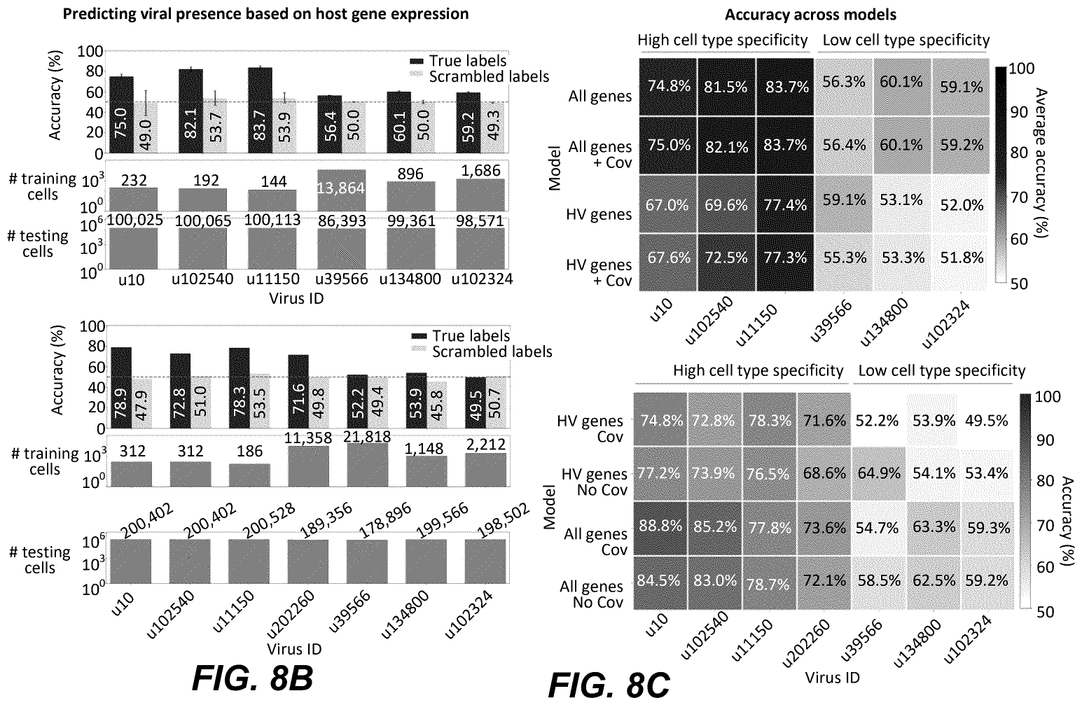

For models that included covariates, donor animal and EVD time point were one-hot encoded and appended to the gene expression training matrix. All models included an intercept. Models were trained with L2 weight regularization using the sklearn.linear_model.LogisticRegression (sklearn v1.0.1) classifier with a maximum of 100 iterations to predict the probability of viral presence at single-cell resolution. Virus-positive cells were assigned class label 1, and virus-negative cells were assigned class label 0. All four possible combinations of two modeling choices (e.g., highly variable versus all genes, and covariates versus no covariates) were tested. The results are shown in FIG. 8C. Accuracy, specificity and sensitivity were calculated for each model on the held-out testing cells (FIG. 16A). A negative control where labels of viral presence and absence for each virus were randomly scrambled in the training data was included in the modeling experiments. For the scrambled labels, the original ratio of virus-positive to virus-negative cells was maintained. All results were averaged across models generated using six different random seeds for parameter optimization and random selection of cells for training and testing.

TABLE 2
THE NUMBERS OF CELLS USED TO TRAIN AND
TEST THE LOGISTIC REGRESSION MODELS

| Virus | Experiment | # training | # virus-neg. | # virus-pos. | # testing | # virus-neg. | # virus-pos. |
|---|---|---|---|---|---|---|---|
| ID | # | cells | training cells | training cells | cells | testing cells | testing cells |
| u10 | 1 | 232 | 116 | 116 | 100,025 | 99,908 | 117 |
| | 2 | 312 | 156 | 156 | 200,402 | 200245 | 157 |
| u102540 | 1 | 192 | 96 | 96 | 100,065 | 99,968 | 97 |
| | 2 | 312 | 156 | 156 | 200,402 | 200245 | 157 |
| u11150 | 1 | 144 | 72 | 72 | 100,113 | 100,041 | 72 |
| | 2 | 186 | 93 | 93 | 200,528 | 200,435 | 93 |
| u202260 | 2 | 11,358 | 5,679 | 5,679 | 189,356 | 5,679 | 183,677 |
| u39566 | 1 | 13,864 | 6,932 | 6,932 | 86,393 | 79,460 | 6,933 |
| | 2 | 21,818 | 10,909 | 10,909 | 178,896 | 167,987 | 10,909 |
| u134800 | 1 | 896 | 448 | 448 | 99,361 | 98,912 | 449 |
| | 2 | 1,148 | 574 | 574 | 199,566 | 198,991 | 575 |
| u102324 | 1 | 1,686 | 843 | 843 | 98,571 | 97,727 | 844 |
| | 2 | 2,212 | 1,106 | 1,106 | 198,502 | 197,396 | 1,106 |

### Enrichment Analysis of Predictive Genes

Two experiments were conducted on top macaque genes. In the first experiment, the top 200 macaque genes were analyzed with the largest positive weights in the regression model trained on highly variable genes with covariates donor animal and time point. In the second experiment, of the top 50 highly variable macaque genes with the largest positive average weights in the regression model, those, for which the standard deviation of the weights was less than half of the lowest weight, were selected. In the second experiment, the model trained on highly variable genes with covariates (e.g., donor animal and time point) was used. The gene weight distributions are shown in FIG. 17A. Approximately half of the macaque Ensembl IDs did not have annotated gene names. Gget was used to translate annotated Ensembl IDs to gene symbols and to perform enrichment analysis on the returned gene symbols using Enrichr against the GEO microbe perturbations database (“Microbe_Perturbations_from_GEO_up”). The reported P values were corrected with the Benjamini-Hochberg method.

TABLE 3
VIRUS ID TO SPECIES-LIKE OPERATIONAL TAXONOMIC UNIT (SOTU) MAPPING

| Virus | | | | | |
|---|---|---|---|---|---|
| ID | Class | Order | Family | Genus | Species |
| u100245 | . | . | Fusariviridae | . | . |

Phylum: Duplornaviricota

| | | | | | |
|---|---|---|---|---|---|
| u110641 | Resentoviricetes | Reovirales | Reoviridae | . | . |
| u10015 | Chrymotiviricetes | Ghabrivirales | Totiviridae | . | . |
| u100111 | | | . | . | . |
| u100215 | | . | . | . | . |

Phylum: Kitrinoviricota

| | | | | | |
|---|---|---|---|---|---|
| u100188 | Alsuviricetes | Martellivirales | Closteroviridae | . | . |
| u100139 | | | . | . | . |
| u100177 | Tolucaviricetes | Tolivirales | . | . | . |
| u100000 | | | Tombusviridae | . | . |
| u100076 | | . | . | . | . |
| u100031 | Alsuviricetes | . | . | . | . |

Phylum: Lenarviricota

| | | | | | |
|---|---|---|---|---|---|
| u100173 | Miaviricetes | Ourlivirales | Botourmiaviridae | Ourmiavirus | . |
| u100644 | Amabiliviricetes | Wolframvirales | . | . | . |
| u100048 | | . | . | . | . |
| u100017 | Allassoviricetes | Levivirales | Leviviridae | . | . |
| u100002 | | | . | . | . |
| u100012 | | . | . | . | . |
| u100074 | Howeltoviricetes | Cryppavirales | . | . | . |
| u100001 | | | Mitoviridae | . | . |
| u100011 | | . | . | . | . |
| u100007 | . | . | . | . | . |
| u100004 | Miaviricetes | Ourlivirales | . | . | . |
| u100153 | | | Botourmiaviridae | . | . |
| u100049 | | . | . | . | . |

Phylum: Negarnaviricota

| | | | | | |
|---|---|---|---|---|---|
| u10 | Monjiviricetes | Mononegavirales | Filoviridae | Ebolavirus | Zaire ebolavirus. |
| u100291 | | | . | . | . |
| u100302 | | | Rhabdoviridae | . | . |
| u100196 | . | . | . | . | . |
| u1001 | Insthoviricetes | Articulavirales | Orthomyxoviridae | Alphainfluenzavirus. | Influenza A virus |
| u103829 | | | | . | . |
| u100733 | . | . | . | . | . |
| u100599 | Ellioviricetes | . | . | . | . |
| u100289 | | Bunyavirales | . | . | . |

Phylum: Peploviricota

| | | | | | |
|---|---|---|---|---|---|
| u27694 | Herviviricetes | Herpesvirales | Herpesviridae | Varicellovirus | Bubaline |
| | | | | | alphaherpesvirus |
| | | | | | 1 |

Phylum: Pisuviricota

| | | | | | |
|---|---|---|---|---|---|
| u102540 | Pisoniviricetes | Nidovirales | Coronaviridae | Alphacoronavirus | . |
| u10240 | | | Arteriviridae | . | . |
| u101227 | | Picornavirales | Picornaviridae | . | . |
| u100296 | | | Dicistroviridae | . | . |
| u102324 | | | Iflaviridae | . | . |
| u100116 | | . | . | . | . |
| u100145 | | Sobelivirales | . | . | . |
| u100024 | Duplopiviricetes | Durnavirales | Picobirnaviridae | . | . |
| u100019 | | | | Picobirnavirus | . |
| u100026 | | | . | . | . |
| u100154 | | | Amalgaviridae | . | . |
| u100093 | | | Partitiviridae | . | . |
| u100251 | | . | . | . | . |
| u100028 | . | . | . | . | . |

Table 3 includes virus ID to species-like operational taxonomic unit (sOTU) mapping for the most highly expressed viruses (also shown in FIG. 6D)). Virus IDs disclosed herein but not included in this list are of unknown taxonomy across all taxonomic ranks.

### Example 1

### Efficient and Accurate Detection of Viral Sequences at Single-Cell Resolution

There are an estimated 10³¹ virions on Earth, which amounts to 10 million virions for every star in the known universe. Viruses inhabit oceans, forests, deserts, and human tissues such as the lungs, blood, and brain. More than 300,000 virus species are estimated to cause human disease. However, only 261 species have been detected in humans. Many of these have been implicated in complex diseases such as heart disease and cancer. Recent studies suggest that viruses also play a major, unexpected role in common neurodegenerative disorders such as Alzheimer's, Parkinson's, and multiple sclerosis. Accurate detection of viral infections is crucial to understanding the impact of viruses on human health.

Of the 261 known disease-causing viruses, 206 fall into the realm of Riboviria, which includes all RNA-dependent RNA polymerase (RdRp)-encoding RNA viruses and RNA-dependent DNA polymerase (RdDp)-encoding retroviruses. Amongst many others, these include Coronaviruses, Dengue viruses, Ebolaviruses, Hepatitis B viruses, influenza viruses, Measles viruses, Mumps viruses, Polioviruses, West Nile viruses and Zika viruses. Existing workflows for detecting viruses using transcriptomics data rely on the availability of pre-assembled reference genomes. Currently, NCBI RefSeq hosts 8,694 Riboviria reference genomes, which is a diminutive fraction of Riboviria viruses. Pioneering work by Edgar et al. leveraged a well-conserved amino acid sub-sequence of the RdRP, called the “palmprint,” to identify RNA viruses from 5.7 million globally and ecologically diverse sequencing samples in the Sequence Read Archive (SRA). This method does not require pre-computed indices, thus allowing alignment to diverged sequences and the discovery of thousands of novel viruses. This effort resulted in a consensus of 296,623 unique RdRP-containing amino acid sequences, referred to as “PalmDB.” Clustering palmprints into sOTUs yielded 146,973 known as well as novel sOTUs. Compared to the 8,694 Riboviria reference genomes currently available on NCBI, this translates to a more than 16× increase in the number of viruses that can be detected. The actual number of virus species that can be detected using the PalmDB is likely even higher due to RdRP sequence conservation across Riboviria. sOTUs, obtained by clustering the palmprints, served to approximate taxonomic assignment and allowed species-level virus identification for 40,392 sequences in the PalmDB.

The increasing use of high-throughput next-generation sequencing (NGS) methods in molecular biology research, agriculture, and healthcare provides an opportunity for the cost-effective surveillance of viral diversity and the investigation of virus-disease correlations. Specifically, single-cell genomics technologies make possible the characterization of viruses at single-cell resolution. A translated alignment tool was provided, which improved the RNA sequencing data preprocessing tool kallisto to support the detection of viral RNA using the amino acid database PalmDB. This is the only method capable of translated alignment, while retaining single-cell resolution. The small size of PalmDB (e.g., 36 MB) enabled efficient detection of orders of magnitude more viruses than detection based on (NCBI) reference genomes. Moreover, operating in the amino acid space yields a method robust to nucleotide point and/or silent nucleotide mutations.

1. Translated Alignment of Nucleotide Sequences to an Amino Acid Reference with Kallisto Enables Efficient, Accurate Detection of at Least 146,973 RNA Viruses in Transcriptomic Data at Single-Cell Resolution

Existing methods to detect viral sequences either (i) rely on and are limited to NCBI reference genomes, (ii) are not able to perform translated alignment of nucleotide data to an amino acid reference, or (iii) are unable to retain single-cell resolution through cell barcode tracking. Table 4 is an overview of available tools for the detection of viral sequences in next-generation sequencing data, and their ability to align to NCBI RefSeq nucleotide genomes, perform translated alignment of nucleotide data against an amino acid reference, and retain single-cell resolution through cell barcode tracking.

TABLE 4
OVERVIEW OF AVAILABLE TOOLS

| | NCBI | PalmDB | Single-cell |
|---|---|---|---|
| Tool | subset | (translated search) | resolution |
| DeepVirFinder | ✓ | x | x |
| FastCiromeExplorer | ✓ | x | x |
| geNomad | ✓ | x | x |
| MARVEL | ✓ | x | x |
| Phigaro | ✓ | x | x |
| VIBRANT | ✓ | x | x |
| viralVerify | ✓ | x | x |
| VirFinder | ✓ | x | x |
| VirSorter2 | ✓ | x | x |
| VirTect | ✓ | x | x |
| DIAMOND | ✓ | ✓ | x |
| Kraken2 | ✓ | ✓ | x |
| Venus | ✓ | x | ✓ |
| Viral-Track | ✓ | x | ✓ |
| VIRTUS2 | ✓ | x | ✓ |

The bulk and single-cell RNA-seq preprocessing tool kallisto was modified to allow translated search. The use of kallisto in combination with PalmDB was also validated for the detection of viral sequences in single-cell and bulk RNA sequencing data. PalmDB is a database of 296,623 unique RdRP-containing amino acid sequences, representing an estimated 146,973 virus species. Compared to the 8,694 Riboviria reference genomes currently available on NCBI, this signified a more than 16,000× increase in the number of viruses that can be detected. FIG. 2A provides an overview of the number of entries per taxonomy in NCBI and PalmDB. The figure can also be viewed interactively at tinyurl.com/4dzwz5ny. The numbers and taxonomic information in FIG. 2A is in Table 5.

TABLE 5
TAXONOMIES AND NUMBERS OF VIRAL SEQUENCES/GENOMES IN FIG. 2A

| | | | | | |
|---|---|---|---|---|---|
| | | | | PalmDB | NCBI |
| Class | Order | Family | Genus | soTUs | RefSeq |

Phylum: Artverviricota

| | | | | | |
|---|---|---|---|---|---|
| Revtraviricetes | Ortervirales | Caulimoviridae | Badnavirus | 1 | 70 |

Phylum: Duplornaviricota

| | | | | | |
|---|---|---|---|---|---|
| Chrymotiviricetes | Ghabrivirales | Chrysoviridae | Alphachrysovirus | 64 | 16 |
| | | | Betachrysovirus | 13 | 10 |
| | | | Chrysovirus | 30 | 2 |
| | | Megabirnaviridae | Megabirnavirus | 3 | 3 |
| | | Quadriviridae | Quadrivirus | 5 | 1 |
| | | Totiviridae | Giardiavirus | 10 | 2 |
| | | | Leishmaniavirus | 32 | 4 |
| | | | Totivirus | 117 | 10 |
| | | | Trichomonasvirus | 59 | 4 |
| | | | Victorivirus | 91 | 29 |
| Resentoviricetes | Reovirales | Reoviridae | Aquareovirus | 18 | 7 |
| | | | Cardoreovirus | 2 | 1 |
| | | | Cypovirus | 20 | 8 |
| | | | Fijivirus | 23 | 7 |
| | | | Orbivirus | 354 | 26 |
| | | | Orthoreovirus | 88 | 11 |
| | | | Phytoreovirus | 12 | 4 |
| | | | Rotavirus | 862 | 10 |
| | | | Seadornavirus | 36 | 3 |
| Vidaverviricetes | Mindivirales | Cystoviridae | Cystovirus | 6 | 7 |

Phylum: Kitrinoviricota

| | | | | | |
|---|---|---|---|---|---|
| Alsuviricetes | Hepelivirales | Alphatetraviridae | Omegatetravirus | 12 | 3 |
| | | Benyviridae | Benyvirus | 10 | 1 |
| | | Hepeviridae | Orthohepevirus | 358 | 3 |
| | | | Piscihepevirus | 2 | 1 |
| | | Matonaviridae | Rubivirus | 20 | 1 |
| | Martellivirales | Bromoviridae | Alfamovirus | 19 | 1 |
| | | | Anulavirus | 18 | 3 |
| | | | Bromovirus | 28 | 7 |
| | | | Cucumovirus | 84 | 4 |
| | | | Ilarvirus | 164 | 25 |
| | | Closteroviridae | Ampelovirus | 112 | 15 |
| | | | Closterovirus | 122 | 16 |
| | | | Crinivirus | 71 | 15 |
| | | | Velarivirus | 29 | 8 |
| | | Endornaviridae | Alphaendornavirus | 92 | 23 |
| | | | Betaendornavirus | 7 | 8 |
| | | Kitaviridae | Cilevirus | 10 | 3 |
| | | Mayoviridae | Idaeovirus | 29 | 3 |
| | | Togaviridae | Alphavirus | 210 | 34 |
| | | Virgaviridae | Furovirus | 20 | 6 |
| | | | Hordeivirus | 15 | 3 |
| | | | Pecluvirus | 2 | 2 |
| | | | Pomovirus | 17 | 5 |
| | | | Tobamovirus | 535 | 37 |
| | | | Tobravirus | 9 | 3 |
| | Tymovirales | Alphaflexiviridae | Allexivirus | 179 | 12 |
| | | | Mandarivirus | 15 | 2 |
| | | | Platypuvirus | 5 | 1 |
| | | | Potexvirus | 394 | 47 |
| | | | Sclerodarnavirus | 1 | 1 |
| | | Betaflexiviridae | Capillovirus | 212 | 5 |
| | | | Carlavirus | 760 | 58 |
| | | | Chordovirus | 13 | 2 |
| | | | Citrivirus | 101 | 1 |
| | | | Divavirus | 7 | 4 |
| | | | Foveavirus | 575 | 9 |
| | | | Prunevirus | 9 | 3 |
| | | | Robigovirus | 48 | 5 |
| | | | Tepovirus | 58 | 2 |
| | | | Trichovirus | 287 | 7 |
| | | | Vitivirus | 366 | 17 |
| | | Deltaflexiviridae | Deltaflexivirus | 196 | 4 |
| | | Gammaflexiviridae | Mycoflexivirus | 13 | 1 |
| | | Tymoviridae | Maculavirus | 73 | 6 |
| | | | Marafivirus | 427 | 13 |
| | | | Tymovirus | 122 | 26 |
| Flasuviricetes | Amarillovirales | Flaviviridae | Flavivirus | 1,807 | 83 |
| | | | Hepacivirus | 9,187 | 27 |
| | | | Pegivirus | 199 | 14 |
| | | | Pestivirus | 401 | 14 |
| Magsaviricetes | Nodamuvirales | Nodaviridae | Alphanodavirus | 137 | 5 |
| | | | Betanodavirus | 38 | 5 |
| | | Sinhaliviridae | Sinaivirus | 73 | 7 |
| Tolucaviricetes | Tolivirales | Carmotetraviridae | Alphacarmotetravirus | 5 | 1 |
| | | Luteoviridae | Enamovirus | 52 | 5 |
| | | | Luteovirus | 171 | 13 |
| | | | Polerovirus | 579 | 36 |
| | | Tombusviridae | Alphacarmovirus | 56 | 8 |
| | | | Alphanecrovirus | 46 | 4 |
| | | | Aureusvirus | 88 | 6 |
| | | | Betacarmovirus | 21 | 4 |
| | | | Betanecrovirus | 143 | 3 |
| | | | Dianthovirus | 24 | 3 |
| | | | Gammacarmovirus | 95 | 4 |
| | | | Machlomovirus | 26 | 1 |
| | | | Panicovirus | 37 | 4 |
| | | | Pelarspovirus | 35 | 7 |
| | | | Tombusvirus | 292 | 16 |
| | | | Umbravirus | 118 | 11 |

Phylum: Lenarviricota

| | | | | | |
|---|---|---|---|---|---|
| Allassoviricetes | Levivirales | Leviviridae | Allolevivirus | 82 | 3 |
| | | | Levivirus | 52 | 2 |
| Amabiliviricetes | Wolframvirales | Narnaviridae | Narnavirus | 113 | 9 |
| Howeltoviricetes | Cryppavirales | Mitoviridae | Mitovirus | 1,640 | 10 |
| Miaviricetes | Ourlivirales | Botourmiaviridae | Botoulivirus | 2 | 2 |
| | | | Ourmiavirus | 1,535 | 3 |
| | | | Scleroulivirus | 13 | 3 |

Phylum: Negarnaviricota

| | | | | | |
|---|---|---|---|---|---|
| Ellioviricetes | Bunyavirales | Arenaviridae | Arenavirus | 4 | 1 |
| | | | Hartmanivirus | 17 | 4 |
| | | | Mammarenavirus | 359 | 38 |
| | | | Reptarenavirus | 117 | 6 |
| | | Fimoviridae | Emaravirus | 45 | 11 |
| | | Hantaviridae | Mobatvirus | 19 | 3 |
| | | | Orthohantavirus | 150 | 39 |
| | | | Thottimvirus | 14 | 2 |
| | | Nairoviridae | Orthonairovirus | 133 | 24 |
| | | Peribunyaviridae | Herbevirus | 13 | 3 |
| | | | Orthobunyavirus | 260 | 83 |
| | | | Pacuvirus | 6 | 4 |
| | | Phasmaviridae | Feravirus | 8 | 1 |
| | | | Jonvirus | 5 | 1 |
| | | | Orthophasmavirus | 7 | 11 |
| | | Phenuiviridae | Bandavirus | 64 | 8 |
| | | | Coguvirus | 40 | 1 |
| | | | Entovirus | 2 | 1 |
| | | | Goukovirus | 2 | 3 |
| | | | Ixovirus | 4 | 2 |
| | | | Mobuvirus | 2 | 1 |
| | | | Phasivirus | 38 | 5 |
| | | | Phlebovirus | 175 | 52 |
| | | | Rubodvirus | 12 | 2 |
| | | | Tenuivirus | 26 | 8 |
| | | | Uukuvirus | 15 | 12 |
| | | | Wenrivirus | 6 | 1 |
| | | Tospoviridae | Orthotospovirus | 96 | 25 |
| Insthoviricetes | Articulavirales | Amnoonviridae | Tilapinevirus | 6 | 1 |
| | | Orthomyxoviridae | Alphainfluenzavirus | 5,729 | 7 |
| | | | Betainfluenzavirus | 349 | 1 |
| | | | Deltainfluenzavirus | 20 | 1 |
| | | | Gammainfluenzavirus | 13 | 1 |
| | | | Isavirus | 12 | 2 |
| | | | Quaranjavirus | 27 | 4 |
| | | | Thogotovirus | 16 | 3 |
| Milneviricetes | Serpentovirales | | | 1 | 6 |
| Monjiviricetes | Jingchuvirales | Chuviridae | Mivirus | 25 | 9 |
| | Mononegavirales | Artoviridae | Peropuvirus | 2 | 6 |
| | | Bornaviridae | Carbovirus | 2 | 2 |
| | | | Orthobornavirus | 34 | 15 |
| | | Filoviridae | Ebolavirus | 79 | 6 |
| | | | Marburgvirus | 9 | 1 |
| | | Lispiviridae | | 62 | 6 |
| | | Mymonaviridae | Sclerotimonavirus | 13 | 2 |
| | | Nyamiviridae | Nyavirus | 5 | 3 |
| | | | Orinovirus | 27 | 1 |
| | | Paramyxoviridae | Aquaparamyxovirus | 2 | 1 |
| | | | Ferlavirus | 5 | 1 |
| | | | Henipavirus | 7 | 5 |
| | | | Hoplichthysvirus | 1 | 3 |
| | | | Jeilongvirus | 5 | 6 |
| | | | Metaavulavirus | 25 | 10 |
| | | | Morbillivirus | 100 | 7 |
| | | | Narmovirus | 2 | 4 |
| | | | Orthoavulavirus | 122 | 8 |
| | | | Orthorubulavirus | 85 | 9 |
| | | | Paraavulavirus | 10 | 2 |
| | | | Pararubulavirus | 12 | 10 |
| | | | Respirovirus | 71 | 7 |
| | | Pneumoviridae | Metapneumovirus | 22 | 2 |
| | | | Orthopneumovirus | 87 | 5 |
| | | Rhabdoviridae | Almendravirus | 15 | 6 |
| | | | Alphanemrhavirus | 2 | 2 |
| | | | Alphanucleorhabdovirus | 2 | 9 |
| | | | Barhavirus | 4 | 2 |
| | | | Caligrhavirus | 9 | 3 |
| | | | Curiovirus | 4 | 4 |
| | | | Cytorhabdovirus | 111 | 19 |
| | | | Dichorhavirus | 11 | 5 |
| | | | Ephemerovirus | 15 | 9 |
| | | | Hapavirus | 18 | 16 |
| | | | Ledantevirus | 26 | 16 |
| | | | Lyssavirus | 161 | 18 |
| | | | Novirhabdovirus | 33 | 4 |
| | | | Nucleorhabdovirus | 60 | 3 |
| | | | Ohlsrhavirus | 19 | 4 |
| | | | Perhabdovirus | 9 | 3 |
| | | | Sawgrhavirus | 4 | 3 |
| | | | Sigmavirus | 12 | 15 |
| | | | Sprivivirus | 22 | 4 |
| | | | Sripuvirus | 13 | 6 |
| | | | Sunrhavirus | 5 | 6 |
| | | | Tibrovirus | 8 | 7 |
| | | | Tupavirus | 7 | 3 |
| | | | Varicosavirus | 16 | 2 |
| | | | Vesiculovirus | 65 | 16 |
| | | Xinmoviridae | Anphevirus | 11 | 2 |
| Yunchangviricetes | Goujianvirales | Yueviridae | Yuyuevirus | 1 | 2 |

Phylum: Nucleocytoviricota

| | | | | | |
|---|---|---|---|---|---|
| Megaviricetes | Imitervirales | Mimiviridae | Mimivirus | 1 | 3 |

Phylum: Peploviricota

| | | | | | |
|---|---|---|---|---|---|
| Herviviricetes | Herpesvirales | Herpesviridae | Muromegalovirus | 1 | 3 |
| | | | Varicellovirus | 1 | 19 |

Phylum: Pisuviricota

| | | | | | |
|---|---|---|---|---|---|
| Duplopiviricetes | Durnavirales | Amalgaviridae | Amalgavirus | 36 | 7 |
| | | | Zybavirus | 2 | 1 |
| | | Hypoviridae | Hypovirus | 48 | 4 |
| | | Partitiviridae | Alphapartitivirus | 254 | 16 |
| | | | Betapartitivirus | 310 | 20 |
| | | | Cryspovirus | 19 | 1 |
| | | | Deltapartitivirus | 155 | 5 |
| | | | Gammapartitivirus | 116 | 9 |
| | | Picobirnaviridae | Picobirnavirus | 3,886 | 7 |
| Pisoniviricetes | Nidovirales | Arteriviridae | Alphaarterivirus | 12 | 1 |
| | | | Betaarterivirus | 1 | 3 |
| | | | Deltaarterivirus | 11 | 1 |
| | | | Epsilonarterivirus | 21 | 3 |
| | | | Etaarterivirus | 13 | 1 |
| | | | Gammaarterivirus | 17 | 1 |
| | | | Iotaarterivirus | 2 | 3 |
| | | | Kappaarterivirus | 8 | 1 |
| | | | Thetaarterivirus | 10 | 2 |
| | | Coronaviridae | Alphacoronavirus | 219 | 24 |
| | | | Betacoronavirus | 567 | 19 |
| | | | Deltacoronavirus | 40 | 10 |
| | | | Gammacoronavirus | 128 | 5 |
| | | Euroniviridae | | 4 | 3 |
| | | Mesoniviridae | Alphamesonivirus | 34 | 12 |
| | | Nanhypoviridae | Sajorinivirus | 3 | 1 |
| | | Roniviridae | Okavirus | 10 | 2 |
| | | Tobaniviridae | Oncotshavirus | 3 | 1 |
| | | | Pregotovirus | 3 | 4 |
| | | | Torovirus | 25 | 7 |
| | Picornavirales | Caliciviridae | Bavovirus | 10 | 1 |
| | | | Lagovirus | 48 | 3 |
| | | | Nebovirus | 10 | 3 |
| | | | Norovirus | 626 | 14 |
| | | | Recovirus | 6 | 1 |
| | | | Salovirus | 13 | 1 |
| | | | Sapovirus | 247 | 7 |
| | | | Vesivirus | 76 | 10 |
| | | Dicistroviridae | Aparavirus | 135 | 6 |
| | | | Cripavirus | 412 | 7 |
| | | | Triatovirus | 74 | 5 |
| | | Iflaviridae | Iflavirus | 433 | 25 |
| | | Marnaviridae | Bacillarnavirus | 7 | 3 |
| | | | Marnavirus | 84 | 1 |
| | | Picornaviridae | Aalivirus | 3 | 1 |
| | | | Aphthovirus | 216 | 5 |
| | | | Avihepatovirus | 32 | 1 |
| | | | Avisivirus | 7 | 4 |
| | | | Bopivirus | 6 | 1 |
| | | | Cardiovirus | 113 | 5 |
| | | | Cosavirus | 38 | 7 |
| | | | Dicipivirus | 4 | 2 |
| | | | Enterovirus | 1,906 | 29 |
| | | | Erbovirus | 4 | 1 |
| | | | Gallivirus | 9 | 2 |
| | | | Harkavirus | 2 | 1 |
| | | | Hepatovirus | 49 | 11 |
| | | | Hunnivirus | 41 | 2 |
| | | | Kobuvirus | 242 | 12 |
| | | | Limnipivirus | 3 | 3 |
| | | | Livupivirus | 2 | 1 |
| | | | Megrivirus | 60 | 9 |
| | | | Mischivirus | 4 | 4 |
| | | | Mosavirus | 2 | 2 |
| | | | Oscivirus | 13 | 2 |
| | | | Parechovirus | 125 | 5 |
| | | | Pasivirus | 152 | 1 |
| | | | Passerivirus | 21 | 2 |
| | | | Poecivirus | 2 | 1 |
| | | | Potamipivirus | 1 | 1 |
| | | | Rabovirus | 8 | 2 |
| | | | Rosavirus | 30 | 4 |
| | | | Salivirus | 16 | 3 |
| | | | Sapelovirus | 64 | 4 |
| | | | Senecavirus | 35 | 1 |
| | | | Shanbavirus | 54 | 2 |
| | | | Sicinivirus | 51 | 2 |
| | | | Teschovirus | 46 | 1 |
| | | | Torchivirus | 7 | 1 |
| | | | Tremovirus | 11 | 2 |
| | | Polycipiviridae | Sopolycivirus | 20 | 6 |
| | | Secoviridae | Cheravirus | 35 | 5 |
| | | | Comovirus | 136 | 11 |
| | | | Fabavirus | 328 | 7 |
| | | | Nepovirus | 466 | 31 |
| | | | Sadwavirus | 14 | 5 |
| | | | Sequivirus | 3 | 3 |
| | | | Torradovirus | 40 | 8 |
| | | | Waikavirus | 59 | 4 |
| | | Solinviviridae | Invictavirus | 10 | 1 |
| | | | Nyfulvavirus | 9 | 1 |
| | | | Husavirus | 24 | 2 |
| | | | Posavirus | 40 | 3 |
| | Sobelivirales | Barnaviridae | Barnavirus | 8 | 1 |
| | | Solemoviridae | Polemovirus | 4 | 1 |
| | | Solemoviridae | Sobemovirus | 233 | 20 |
| Stelpaviricetes | Patatavirales | Potyviridae | Arepavirus | 6 | 2 |
| | | | Bymovirus | 49 | 6 |
| | | | Ipomovirus | 58 | 7 |
| | | | Macluravirus | 94 | 10 |
| | | | Poacevirus | 31 | 3 |
| | | | Potyvirus | 2,128 | 180 |
| | | | Roymovirus | 2 | 2 |
| | | | Rymovirus | 24 | 3 |
| | | | Tritimovirus | 39 | 6 |
| | Stellavirales | Astroviridae | Avastrovirus | 235 | 5 |
| | | | Mamastrovirus | 1,006 | 37 |

Phylum: Uroviricota

| | | | | |
|---|---|---|---|---|
| Caudoviricetes | Caudovirales | Myoviridae | 1 | 77 |

Phylum: N/A

| | | | |
|---|---|---|---|
| Birnaviridae | Aquabirnavirus | 1 | 5 |
| | Avibirnavirus | 5 | 1 |
| | Botybirnavirus | 11 | 3 |
| Birnaviridae | Entomobirnavirus | 1 | 1 |
| Fusariviridae | | 390 | 14 |
| | Negevirus | 57 | 8 |
| Permutotetraviridae | | 32 | 2 |
| Polymycoviridae | Polymycovirus | 22 | 7 |
| | Unclassified | 97,106 | 0 |
| | Unclassified sp. | 1,598 | 313 |

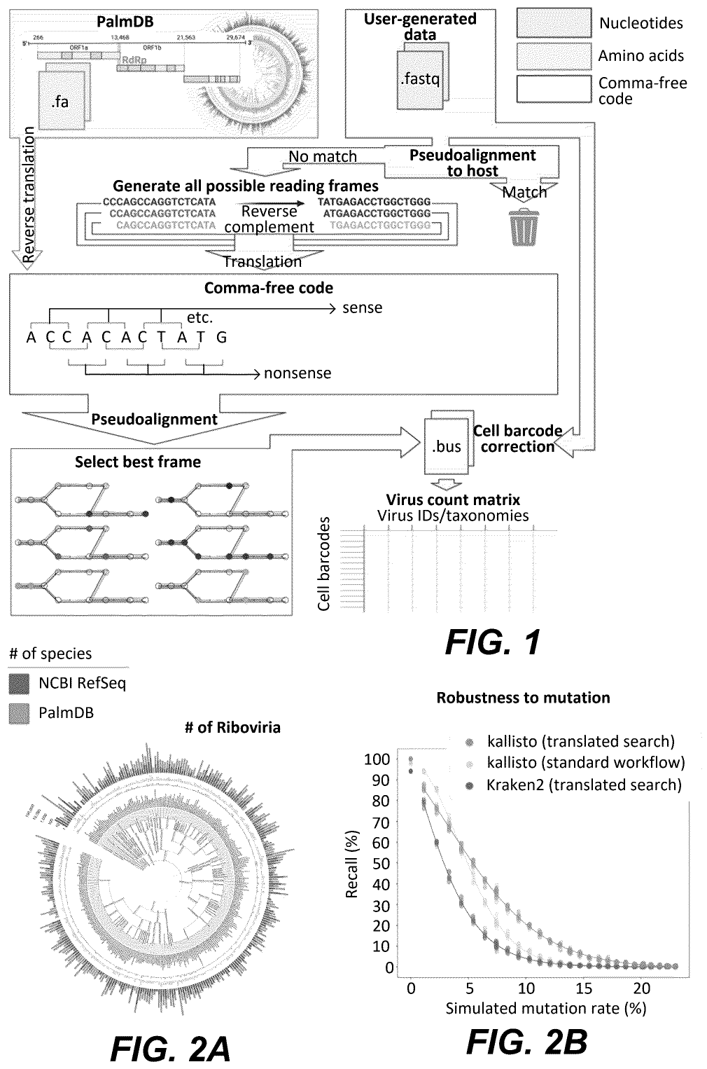

The translated alignment was performed by first reverse translating the amino acid reference sequences (e.g., sequences in PalmDB) and all possible reading frames, including three forward and three reverse, of the nucleotide sequencing reads to comma-free code, following the workflow shown in FIG. 1. A comma-free code is a set of k-letter “words” selected such that any off-frame k-mers formed by adjacent letters do not constitute a “word,” and would thus be interpreted as “nonsense,” as illustrated in FIG. 1. All possible words derived from a k-mer can be calculated using the equation below:

$${W}_{k} ( n ) = \frac{1}{k} {\sum }_{d ∖ k} μ ( d ) {n}^{\frac{k}{d}} \qquad ( 3 )$$

Equation (3) is used for k-mers with k=1, 3, 5, 7, 9, 11, 13 or 15, in which n is the number of letters in alphabet (e.g., 4 nucleotides), k is the number of letters per “word” (e.g., k=3 in triplet code) and μ(d) is Mobius function of divisors d of k. For k=3 (a triplet code) and 4 letters (e.g. ‘A,’ ‘T,’ ‘C’ and ‘G’), this results in exactly 20 possible words, which equals the number of amino acids specified by the universal genetic code, as calculated below using Equation (3):

$${W}_{3} ( 4 ) = \frac{1}{3} ( {4}^{3} - 4 ) = 20 \qquad ( 4 )$$

Due to the serendipity of these numbers, Crick et al. hypothesized the genetic code to be a comma-free code in 1957. The impossibility of off-frame matches makes comma-free codes highly appropriate for translated alignment (FIG. 18A-FIG. 18E). The de Bruijn graph generated from the reverse translated PalmDB sequences grouped viruses of the same taxonomies, indicating that within-taxonomy similarity was conserved in comma-free space as expected (FIG. 12C). Finally, the six reading frames of the sequencing reads translated to comma-free code were pseudoaligned to the reference sequences reverse translated to comma-free code. If several reading frames of the same read produced alignments, the best frame was chosen (FIG. 1).

The workflow could be executed in three lines of code, and computational requirements did not exceed those of a standard laptop (FIG. 1). Below is a set of exemplary code used in the workflow illustrated in FIG. 1:
#1. Install kb-python (optional: install gget to fetch the host genome and/or transcriptome) Pip install kb-python gget
#2. Create reference index
#Optional: masking of host genomic and/or transcriptomic sequences using the D-list
#Single-thread runtime: 1.5 h; Max RAM: 4.4 GB; Size of generated index: 593 MB
#Without D-list: Single-thread runtime: 3.5 min; Max RAM: 3.9 GB; Size of generated index:

```
592 MB
kb ref \
  --aa \
  --d-list $(gget ref -ftp -w dna homo_{—}sapiens) \
  -i index.idx --workflow custom \
  palmdb_{—}rdrp_{—}seqs.fa
# 3. Align sequencing reads and generate count matrix
# Single-thread runtime: 1.5 min per 1 million sequences; Max RAM: 2.1
GB
kb count \
  --aa \
  -i index.idx -g palmdb_{—}clustered_{—}t2g.txt \
  --parity single \
  -x default \
  user_{—}data.fastq.gz
```

The workflow illustrated in FIG. 1 and code above used a novel argument ‘--aa’ to activate the translated search alignment. In this exemplary workflow, the reference file (e.g., “palmdb_rdrp_seqs.fa”) consisted of the database (e.g., PalmDB) comprising amino acid sequences of RdRP. The “palmdb_clustered t2g.txt” file grouped virus IDs with the same taxonomy across all main taxonomic ranks like transcripts of the same gene. Both files are available at tinyurl.com/4wd33rey. During the generation of the reference index with “kb ref,” the D-list option can be used to mask host genomic and/or transcriptomic sequences. For example, in the exemplary code above, human genomic sequences fetched from Ensembl using gget were masked using the D-list. The reference index/data (e.g., PalmDB) only needed to be generated once. The precomputed PalmDB reference indices for human and mouse hosts are available at tinyurl.com/aaxyy8v8. Following the generation of a reference index, the sequencing reads were pseudoaligned to the reference index, and a count matrix was generated using the “kb count” command. The “-x” argument was used to define the sequencing technology. In the example code above, the minimum required user input is marked in bold (amino acid space) and italic (nucleotide space). Building on kallisto's versatility, the workflow disclosed herein was compatible with all state-of-the-art single-cell and bulk RNA sequencing methods, including but not limited to 10× Genomics, Drop-Seq., SMART-Seq, SPLiT-Seq including Parse Biosciences, and spatial methods (e.g., Visium).

Validation testing was performed using different bulk and single-cell RNA sequencing datasets with known infections with severe acute respiratory syndrome coronavirus 2 (SARS-CoV-2) or Zaire ebolavirus (ZEBOV). In these tests, translated search with kallisto and PalmDB was able to detect the viral RNA and correctly assign species-level taxonomy at counts correlating with viral loads measured by RT-qPCR or RNA-ISH, regardless of the technology used to generate the data (FIG. 3A). FIG. 3B provides an overview of the accuracy of the taxonomic assignment across all available taxonomic ranks after reverse translated RdRP sequences were aligned to the PalmDB with kallisto translated search. The number of sequences at each taxonomic rank is summarized in Table 6. At the species level, 96.76%-96.82% of 296,561 sequences were assigned the correct taxonomy (species panel of FIG. 3B), 0.007%20.0082 were assigned an incorrect taxonomy, 0.37%-0.38% could not be unambiguously matched to a single virus (e.g., multimapped), and 2.79%-2.86% were not aligned. This confirms that the sequence transformation introduced by the kallisto translated search pipeline retained taxonomic assignments with up to species-level specificity.

TABLE 6
NUMBER OF SEQUENCES AT EACH TAXONOMIC RANK IN FIG. 3B

| | # | | # | | # |
|---|---|---|---|---|---|
| Phylum | sequences | Class | sequences | Order | sequences |
| Pisuviricota | 56,104 | Pisoniviricetes | 23,505 | Nidovirales | 1,588 |
| Negarnaviricota | 14,297 | Monjiviricetes | 4,116 | Mononegavirales | 3,202 |
| Kitrinoviricota | 35,349 | Flasuviricetes | 11,882 | Amarillovirales | 11,811 |
| Duplornaviricota | 6,712 | Alsuviricetes | 10,766 | Hepelivirales | 543 |
| Unknown | 99,243 | Resentoviricetes | 1,586 | Reovirales | 1,534 |
| Lenarviricota | 84,847 | Insthoviricetes | 6,536 | Picornavirales | 15,410 |
| Nucleocytoviricota | 2 | Tolucaviricetes | 8,503 | Articulavirales | 6,466 |
| Peploviricota | 2 | Stelpaviricetes | 4,853 | Tolivirales | 6,570 |
| Artverviricota | 2 | Magsaviricetes | 2,427 | Sobelivirales | 970 |
| Uroviricota | 3 | Ellioviricetes | 3,186 | Patatavirales | 2,561 |
| | | Unknown | 108,898 | Nodamuvirales | 1,333 |
| | | Howeltoviricetes | 18,957 | Bunyavirales | 2,736 |
| | | Duplopiviricetes | 22,053 | Unknown | 144,946 |
| | | Chrymotiviricetes | 4,725 | Martellivirales | 3226 |
| | | Megaviricetes | 1 | Cryppavirales | 13,362 |
| | | Amabiliviricetes | 5,905 | Tymovirales | 5,676 |
| | | Miaviricetes | 11,879 | Durnavirales | 20,178 |
| | | Allassoviricetes | 46,591 | Ghabrivirales | 3,868 |
| | | Herviviricetes | 2 | Imitervirales | 1 |
| | | Vidaverviricetes | 23 | Wolframvirales | 2,144 |
| | | Revtraviricetes | 1 | Stellavirales | 2,048 |
| | | Caudoviricetes | 2 | Ourlivirales | 10,455 |
| | | Yunchangviricetes | 161 | Levivirales | 35,514 |
| | | Milneviricetes | 3 | Herpesvirales | 2 |
| | | | | Mindivirales | 21 |
| | | | | Ortervirales | 1 |
| | | | | Caudovirales | 2 |
| | | | | Goujianvirales | 158 |
| | | | | Jingchuvirales | 334 |
| | | | | Serpentovirales | 1 |

Moreover, kallisto translated search with PalmDB correctly identified sequences that originate from the RdRP gene. To this end, a subset of 100,000,000 reads obtained using Seq-Well sequencing of macaque peripheral blood mononuclear cell (PBMC) samples obtained at 8 days post-infection with ZEBOV was selected. The reads were aligned to the PalmDB amino acid sequences with kallisto translated search. The reads were also aligned to the complete ZEBOV nucleotide genome using Kraken2, which performed standard nucleotide alignment. Aligned reads from both alignments were extracted and realigned to the ZEBOV genome using bowtie2. A BAM file was created using SAMtools and the alignment was subsequently visualized NCBI Genome Workbench. The visualized alignments are shown in FIG. 10, confirming that kallisto translated search accurately and comprehensively detected ZEBOV RdRP sequences.

The robustness of the translated search method disclosed herein to single nucleotide mutations was also tested. Single nucleotide mutations occur at a relatively high rate in RNA viruses of up to 10-4 substitutions per nucleotide site per cell infection. Random single nucleotide base substitutions were added to 676 ZEBOV nucleotide RdRP sequences identified during the alignment described above. Then, the frequency of correct taxonomic classification (recall percentage) by kallisto translated search was assessed, in comparison to the current state-of-the-art translated search tool, Kraken2 (translated search). Kallisto translated search correctly recalled up to 27.5%-30% more viral RdRP sequences than Kraken2 (translated search) (FIG. 2B). Moreover, kallisto translated search was more robust than aligning to the complete nucleotide genome with the standard kallisto workflow at mutation rates >4% (FIG. 2B), which emphasizes the advantage of operating in the amino acid space. While the Kraken2 translated search and the kallisto standard workflow were given only the correct virus as a reference (e.g., ZEBOV), kallisto translated search had to distinguish between all viruses contained in the PalmDB and identify the correct taxonomy. Kallisto translated search was able to maintain >90% precision in the genus-level taxonomic assignment at mutation rates up to 12% (FIG. 12A).

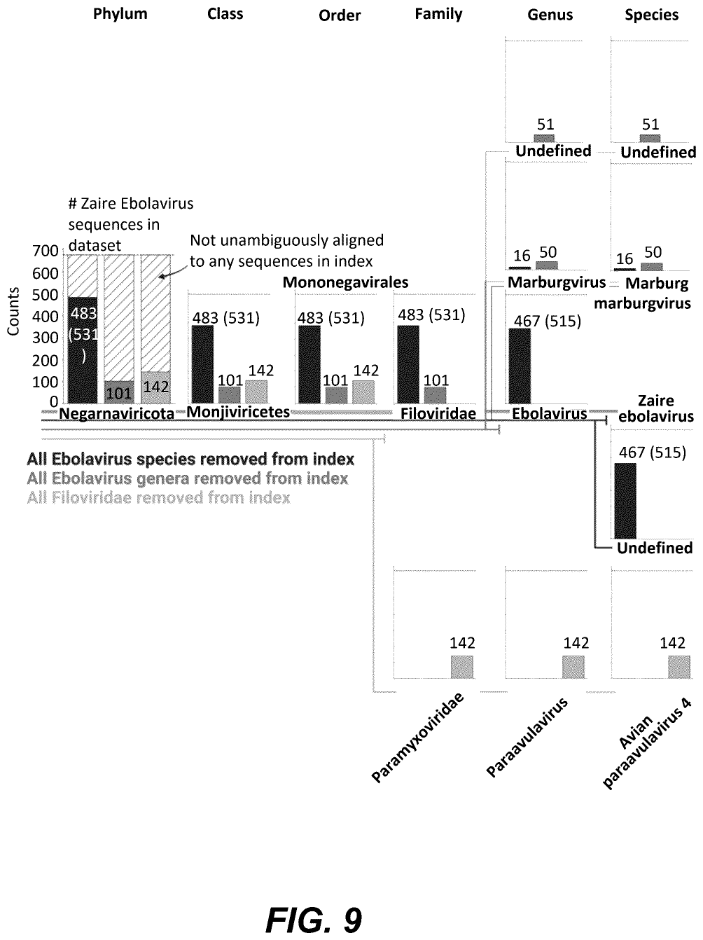

Moreover, viral species not included as sOTUs in the reference PalmDB database could also be detected based on the conservation of the RdRP gene. To confirm this, all Ebola virus species, all Ebolavirus genera and all members of the Filoviridae family were removed from the reference. Subsequently, the 676 ZEBOV RdRP sequences obtained by Seq-Well sequencing were aligned. In each scenario, a subset of sequences aligned to the nearest remaining relative based on the main taxonomic rank (FIG. 9). This confirmed that kallisto translated search detected the highly conserved RdRP of a large number of viral species, beyond the number of sequences in the PalmDB database, while still providing reliable sOTU-based taxonomic assignment of lower-rank taxonomies.

### 2. Read and Virus Filtering

A common problem that arises during the identification of microbial sequences is the cross-species contamination of reference genome databases, such as the ubiquitous contamination of bacterial genomes with human DNA. This can lead to the misclassification of host reads as bacterial or viral, suggesting the presence of microbes that were not truly present. The misclassification of host reads as viral can be prevented by removing host reads prior to the alignment to the viral reference. However, conservatively removing host reads would also remove sequences of endogenous viral elements, which are very abundant in vertebrate genomes and may lead to the removal of viral sequences that were truly present. Therefore, the filtering methods disclosed herein achieved two goals: (i) removing host reads to prevent the misclassification of host reads as viral while (ii) comprehensively identifying the virome within a sample.

In some instances, it is impossible to unambiguously determine whether a read originated from the host or a virus/microbe during the alignment. For example, an analysis of cancer microbiomes identified the presence of several bacterial genera. While the reanalysis of this data using highly conservative host sequence masking led to a significant decrease in the number of reads identified for each bacterial genus, many bacterial genera remained identified. Conservative host masking led to the removal of sequences originating from a confirmed viral infection, as was the case for ZEBOV here (FIG. 4C and FIG. 5A). Hence, it was unclear whether removing all ambiguous reads (e.g., reads that aligned with both host genome/transcriptome and reference sequences) prior to downstream analysis was correct or resulted in biologically relevant data being thrown out. Thus, easy-to-use, flexible and efficient sequence masking methods were developed, which allowed the identification of reads that aligned to both host and virus/microbe while also preserving these reads for downstream analysis. These masking methods were used in combination with PalmDB for the identification of viral sequences. However, they can be applied to mask any nucleotide or amino acid reference and are able to retain single-cell resolution.

The impact of different host masking options on the resulting virome was first evaluated. Kallisto translated search with PalmDB was used to map the virus profiles of peripheral blood mononuclear cell (PBMC) RNA sequencing samples from 19 rhesus macaques and applied different host masking workflows. The approach to masking host versus microbe reads and the handling of overlap between reference sequences can affect the downstream result. For example, sequences with varying sizes of virus-host overlap, sequences that span the junction of two exons, and entirely ambiguous sequences influenced the outcome of the masking and resulted in highly variable results depending on the method used (FIG. 4A and FIG. 13). Depending on the research question and design, any one or a combination of different masking options might be appropriate. The following masking options, listed from least to most conservative, were explored:

### No Mask

The sequencing reads were aligned to the PalmDB with kallisto translated search without masking or previously removing host sequences. For the macaque PBMC dataset, this masking option resulted in 243 distinct sOTUs detected (FIG. 4A).

### D-List Genome+Transcriptome

To incorporate host read masking into the kallisto workflow disclosed herein, the reads were quantified while masking the host genome and transcriptome using an index created with the D-list (distinguishing list) option. This option identifies sequences that are shared between a target transcriptome (e.g., RdRP amino acid sequences) and a secondary genome and/or transcriptome (e.g., host genome and/or transcriptome). k-mers flanking the shared sequence on either end in the secondary genome were added to the index de Bruijn graph. During pseudoalignment, the flanking k-mers were used to identify reads that originated from the secondary genome but would otherwise be erroneously attributed to the target transcriptome due to the spurious alignment to the shared sequences. In the examples disclosed herein, the target transcriptome consisted of the viral RdRP amino acid sequences contained in the PalmDB, and the secondary genome consisted of transcriptomic and genomic macaque and dog nucleotide sequences. When combining D-list with translated search, the secondary genome was translated to comma-free code in all six possible reading frames (FIG. 4B). This masking option can be easily added to the kallisto translated search workflow without any additional commands (FIG. 1). Masking the host transcriptome and genome with D-list resulted in 150 distinct sOTUs detected (FIG. 4A). Note that masking both the transcriptome and the genome, or either one would generate different results because masking only the genome would not mask sequences that span exon-exon junctions (FIG. 4A and FIG. 13).

Host Read Capture with Kallisto

To imitate prior alignment to the host genome, as performed with bwa, within a simple, efficient kallisto workflow, all reads that pseudoaligned to the host transcriptome with kallisto were captured. Masking by capturing these host reads resulted in the same number of distinct sOTUs detected as masking with D-list (FIG. 4A).

Host Read Capture with Kallisto+D-List Genome+Transcriptome

Although masking with D-list and capturing reads that aligned to the host transcriptome resulted in the same number of distinct sOTUs detected, the two methods masked different reads and resulted in different virus profiles (FIG. 5A and FIG. 13). The number of positive cells obtained by each masking workflow is shown in FIG. 5A and in the Table 7 below. Therefore, the D-list and host read capture masking approaches were combine to achieve a conservative result similar to that achieved by prior alignment with bwa. In this approach, the sequencing reads were aligned to the PalmDB index with a D-list containing the host genome and transcriptome, and subsequently reads that pseudoaligned to the host transcriptome were captured. Combining the D-list and host read capture masking options reduced the number of detected sOTUs to 80 (FIG. 4A).

TABLE 7
THE NUMBER OF POSITIVE CELLS OBTAINED
BY EACH MASKING WORKFLOW

| | | | | Host read capture | |
|---|---|---|---|---|---|
| | | | Host read | with kallisto + | Prior |
| | | D-list genome + | capture with | D-list genome + | aligment to |
| Virus ID | No mask | transcriptome | kallisto | transcriptome | host with bwa |
| u10 | 314 | 315 | 313 | 314 | 305 |
| (ZEBOV) | | | | | |
| u202260 | 218,334 | 18,531 | 216,649 | 17,649 | 13,903 |
| u103829 | 12,075 | 12,087 | 9,645 | 9,621 | 5,330 |
| u102324 | 2,517 | 2,352 | 1,677 | 1,546 | 401 |
| u102540 | 323 | 334 | 302 | 313 | 180 |
| u1001 | 465 | 267 | 392 | 211 | 81 |
| u39566 | 37,401 | 224 | 24,518 | 172 | 0 |
| u11150 | 189 | 191 | 180 | 181 | 0 |
| u41991 | 68,447 | 0 | 59,834 | 0 | 203 |
| u164445 | 182 | 185 | 0 | 0 | 0 |
| u162905 | 116 | 0 | 116 | 0 | 0 |
| u149397 | 11,274 | 0 | 0 | 0 | 0 |

Prior Alignment to Host with Bwa

The sequencing reads were aligned to the macaque and dog genomes using the highly sensitive alignment algorithm bwa and removed all reads that aligned anywhere in the host genomes before alignment to PalmDB with kallisto translated search. This achieved very conservative masking of the host genome. However, this workflow was complex, time-consuming and computationally expensive. For example, it took about 4.5 days using 60 cores for the macaque ZEBOV PBMC dataset. This workflow resulted in the detection of 53 distinct sOTUs (FIG. 4A).

There are inherent differences between these masking methods, which are illustrated in FIG. 4A. Although the genome was passed to the software, the standard kallisto workflow built an index based on the host transcriptome, not the entire host genome, since for genomes as large as the macaque genome, building the index on the entire genome would require a large amount of memory. Hence, masking by capturing reads that pseudoaligned to the host with kallisto only captured host reads from mature mRNA molecules. If the D-list was passed both the transcriptome and the genome, mature and nascent RNA molecules as well as RNA molecules originating from intergenic regions were all masked. The D-list index avoided excessive memory requirements by restraining the index to distinguishing sequences between viral and host sequences. As a result, reads that contained non-flanking host and viral sequences was not filtered. Moreover, the D-list favored viral assignment in the case of an entirely ambiguous read. In other words, if the read aligned to both the host genome and RdRP sequences in PalmDB, the D-list assigned it as viral sequence. Neither of these issues applied to masking with bwa, since the alignment with bwa was performed against the host genome. Since bwa used a smaller seed length than kallisto's default k-mer size of 31, bwa provided more sensitive alignment of sequencing reads against the host genome and the most stringent filtering.

To confirm that reads identified as viral were not misaligned macaque reads, randomly selected sequencing reads from 11 virus IDs were extracted and aligned against the nucleotide sequence database with BLAST+(FIG. 5B). The reads associated with virus IDs identified by all masking workflows (e.g., u202260, u103829, u102324, u102540 and u1001) BLAST-aligned with relatively low coverage and identity to several superkingdoms, including viruses. For u202260, approximately two third of the extracted reads yielded no BLAST results. Given that the majority of RdRP sequences in the PalmDB originated from unknown viruses lacking reference genomes in NCBI RefSeq, it was expected that these sequences would not yield confident BLAST results. However, given the comprehensiveness of the macaque genome, misaligned macaque sequences should BLAST to the macaque genome with high coverage and identity. The next two virus IDs, u39566 and u1150, were filtered out by the bwa workflow and did not BLAST to the viruses superkingdom. However, their BLAST results displayed relatively low coverage and identity to macaque genome, which would not be expected from macaque sequences. Below, further evidence was provided that u1150 sequences might have originated from an ongoing viral infection. This was likely an instance where filtering with bwa was too conservative and threw out viral sequences. u41991 was identified as viral by the bwa workflow but filtered out by the D-list+host capture workflow. Based on the BLAST results for u41991, which included high coverage and identity matches for eukaryotes, it was likely that filtering was the appropriate action. u164445 and u162905 were filtered by either capturing the host reads or using the D-list, respectively, and BLAST to eukaryotes with high coverage and identity, suggesting that a combination of the two methods leads to more robust results. Finally, sequences identified as u149397, which were filtered by all masking options and were only retained without masking, BLAST-aligned to eukaryotes with high coverage and identity.

Separately from exploring the results of different read masking options, virus filtering was also investigated. Host read capture with kallisto generated two separate count matrices: One contained counts for reads that were solely viral, and a second contained counts for viral reads that also pseudoaligned to the host transcriptome. The distinction between filtering reads and filtering viruses becomes evident when examining the two count matrices: for the macaque PBMC dataset, most viruses found in ≥0.05% of cells had at least some reads that also mapped to the host transcriptome, including 2 reads for ZEBOV (FIG. 4C). Moreover, aligning without host masking often led to the detection of more positive cells (FIG. 13). Hence, naive masking of reads can reduce the detection sensitivity of viruses that seemed to be truly present. The masking workflows disclosed herein facilitated the identification of viruses with a high likelihood of being truly present based on conservative host read masking, while also obtaining unmasked reads for these viruses to prevent the decrease in sensitivity inherent to masking host reads. This approach was applied when training the logistic regression models described below in Example 2 to minimize the occurrence of false viral absence.

### 3. Advantages of Translated Alignment and Applications

A method for extracting a “virome” modality from any bulk or single-cell RNAseq data is disclosed herein, by leveraging a new method that mapped and quantified species-level viral RdRP sequences against an amino acid reference. The method was built on the existing alignment software kallisto and bustools and expanded them for translated alignment by reverse translating both the amino acid reference and the nucleotide sequencing reads into a common, non-redundant comma-free code. While the kallisto translated search in combination with PalmDB was validated for the identification of viral RNA, the novel workflow can be applied in combination with any amino acid reference. Kallisto translated search permitted the alignment of nucleotide sequencing data to any amino acid reference at single-cell resolution. For example, amino acid sequences of antimicrobial peptides can be used as a reference to identify these peptides in bulk and single-cell RNA sequencing data. Moreover, amino acid transcriptomes of homologous species can be used as a reference for species with missing or incomplete reference genomes. Operating in the amino acid space can increase similarity between amino acid sequences of species due to the robustness to single-nucleotide mutations.

Kallisto translated search in combination with PalmDB was validated for the detection and identification of viral RNA from at least 100,000 (e.g., 146,973) virus species in next-generation sequencing data at single-cell resolution. The number of viruses expected to cause human infectious disease is eclipsed by the comparatively few viruses with complete reference genomes and the even smaller number of viruses that have been detected in humans. It is important to monitor the presence of viruses in the human population, both to prevent pandemic outbreaks and to further understand the role of viruses in various diseases. Such monitoring and novel virus discovery was performed using single-cell RNA-seq data. Moreover, a platform for characterizing omnipresent virus-like sequences associated with different environments, hosts and laboratory reagents is provided herein.

The virus count matrix, which was obtained using kallisto translated search in combination with PalmDB, is an entirely new modality. This matrix was sparse with relatively low molecule counts per cell (FIG. 11E). While using the highly conserved RdRP to identify viruses made the workflow disclosed herein very efficient and was the key to being able to detect up to 10¹² distinct viruses, RdRP RNA only makes up about 1% of the total viral RNA present in the sequencing data analyzed here (FIG. 10), resulting in the sparsity of the virus count matrix. Furthermore, this number varied between virus species and sequencing technology, making it difficult to define a general detection limit. To normalize this sparse and low-count matrix, the virus count matrix was binarized such that each cell was either positive or negative for each virus. Given the low counts, a high occurrence of false negatives in the virus count matrix was expected, while the confidence in positive cells was high. However, relationships between viral presence and host gene signatures can be learned regardless.

A common problem in the identification of microbial sequences is the misidentification of host sequences as microbial. The PalmDB was not a curated database, and it is possible that some virus-like sequences in the PalmDB were not derived from viruses. In addition, differentiating between ongoing infections, reagent or sample contamination, cell-free RNA contamination, endogenous retroviruses and widespread latent infections was a challenge. The kallisto translated search method computed both the virus count matrix and the host gene expression matrix at single-cell resolution, providing unique opportunities for parallel analysis of viral signatures and their effect on host gene expression. Different approaches were described to evaluate the nature of viral sequences identified by kallisto translated search, including taxonomic assignment of viruses based on the sOTU, analysis of viral tropism, extraction and BLAST alignment of raw sequencing reads identified as viral, and using a sample spike-in to categorize viruses into shared and sample-specific viruses. Moreover, different workflows to mask the host genome and/or transcriptome were described and evaluated, allowing different levels of conservativeness and the quantification of sequencing reads that aligned to viral RdRPs as well as the host transcriptome. Notably, the efficacy of masking the host genome and/or transcriptome depended on the quality and comprehensiveness of the genome/transcriptome. In this example, the majority of host sequences originated from rhesus macaque, which has a very comprehensive genome assembly. Finally, logistic regression models, which are described in details in Example 2 below, were trained to predict viral presence at the single-cell level based on host gene expression, achieving high accuracy indicative of an ongoing viral infection or clearance. The results showed that it was beneficial to combine multiple of these approaches, which were validated and described in details, for the interpretation of the presence of virus-like sequences.

Focusing on the RdRP produced biases between virus species with varying life cycles, depending on the sequencing technology used. The genome of many negative-strand RNA (−ssRNA) was replicated as well as transcribed. Transcription produced short, often polyadenylated mRNA products, which were captured and sequenced, including the RdRP. In contrast, the genome of many positive-strand RNA (+ssRNA) viruses undergoes replication, but not transcription. Instead, the genome is translated into polyproteins, which are subsequently cleaved. While +ssRNA virus genomes are often polyadenylated and hence are captured by polyA capture-dependent single-cell RNA sequencing technologies, sequencing ˜100 bases from the polyA-tail using a poly(T) primer may not capture the RdRP if it is located too far from the polyA-tail. A schematic overview of the SARS-CoV genome is in FIG. 1. In this scenario, the RdRP of +ssRNA viruses can be captured by bulk RNA sequencing and random hexamer primers in single-cell RNA sequencing (FIG. 12B). Hence, sequencing using random hexamer primers overcomes the virus life cycle-dependent bias for single-cell technologies. Many novel sequencing technologies, including Parse Biosciences SPLiT-Seq, employ random hexamer primers to produce full-coverage sequencing and overcome biases introduced by poly(T) primers. The use of random priming in sequencing may continue to increase. It is worth noting that, depending on the technology, intra-genomic sequences of +ssRNA viruses can be captured by poly(T) primers nonetheless due to mis-priming. Even with random priming, many biases can remain. For example, any viral RNA that is not polyadenylated may not be captured efficiently by single-cell sequencing technologies that rely on polyA capture.

The wide implementation of kallisto translated search in the analysis of next generation sequencing data can be advantageous in identifying the presence of viral RNA, as well as informing the experimental design of research aiming to identify microbial reads from RNA sequencing data. Several experimental design choices were described that greatly impact the results of microbial read quantification, such as the sequencing primer design and sample spike-ins. The masking workflows described herein and the associated challenges are applicable to any metagenomics analysis beyond the identification of viral reads, and the workflows described herein can be easily applied to nucleotide references, such as a 16S database for the characterization of the human gut microbiome.

### Example 2

### Prediction of Viral Presence Based on Host Gene Expression

### 1. The Presence of Novel Viruses Perturbs Host Gene Expression in Macaque Blood Cells, Allowing Prediction of Viral Presence Based on Host Gene Expression at Single-Cell Resolution

Kallisto translated search and the PalmDB were used to map the viral profiles of PBMC samples from 19 rhesus macaques sequenced at different stages of Ebola virus disease (EVD) (FIG. 6A) at single-cell resolution. The dataset consisted of 30,594,130,037 reads in total. After alignment to both the host genome using the standard kallisto workflow and PalmDB using kallisto translated search with D-list+host capture masking, and quality control using the host count matrix (FIG. 11A), 202,525 PBMCs were retained. The Leiden algorithm was used to partition the PBMC transcriptomes into 18 clusters of similar macaque gene expression, of which 16 could be assigned cell types based on common marker genes (FIG. 11D).

The obtained cell types, their marker gene expression and relative abundance over time were consistent with the results reported by Kotliar et al., including the emergence of a cluster of immature neutrophils and decreased lymphocyte abundance, especially natural killer cells, during EVD (FIG. 7A). While density-based PBMC isolation typically removed neutrophils, immature neutrophils were less dense than mature neutrophils and were co-isolated with PBMCs during infections. Clusters of the same cell type were often separated by time point (FIG. 7A), indicating changes in macaque gene expression within the same cell type over the course of the EVD. This was in agreement with results obtained by mass cytometry in the report by Kotliar et al.

ZEBOV count data from this analytic workflow was also consistent with previously reported results. Since only a small fraction of the RNA molecules in these tissue samples were viral, of which only the RdRP was detected, the measured absolute RNA counts for any one virus per cell were low (FIG. 11E). To facilitate analysis, the virus count matrix was converted into a binary matrix where each virus was recorded as being either present or not present in each cell. This approach has been previously validated for sparse single-cell RNA, specifically viral, sequencing data, and prevented the need for further normalization by individual cellular viral load, which may introduce biases. The presence of virus in each cell was then used to determine the viral abundance among populations of cells composed of clusters, cell types or tissues. First, the binary virus count matrix was used to validate the detection of ZEBOV. Samples obtained during incubation displayed the highest abundance of ZEBOV-positive cells, and ZEBOV-positive cells remained detectable at all following time points (FIG. 7B, top panel). These trends were consistent with the results reported by Kotliar et al.

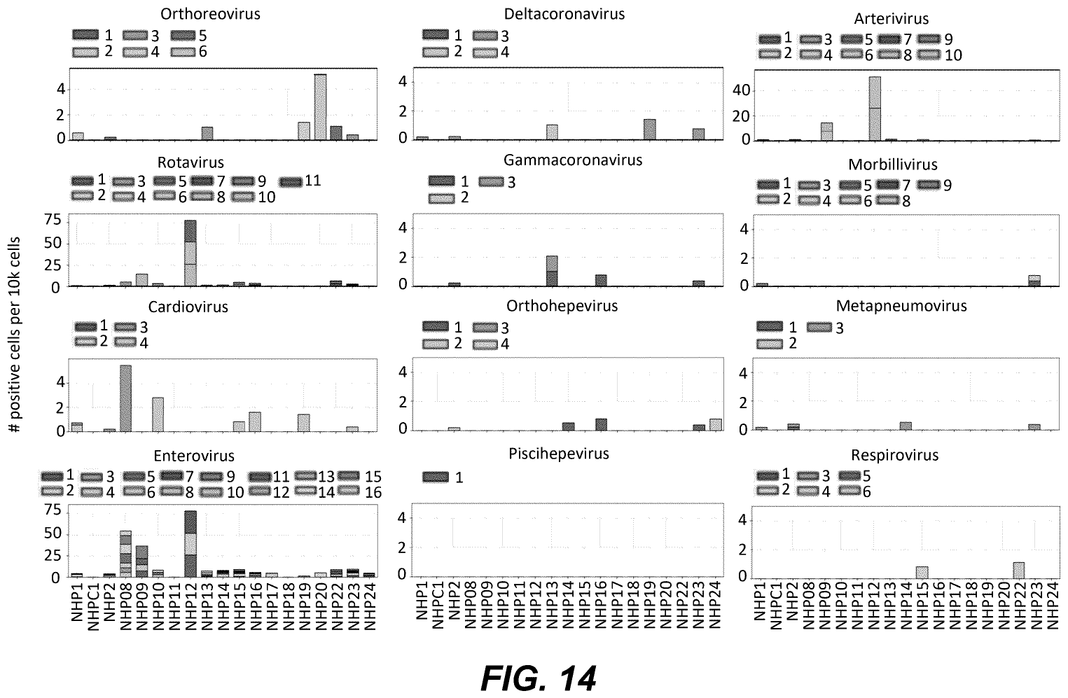

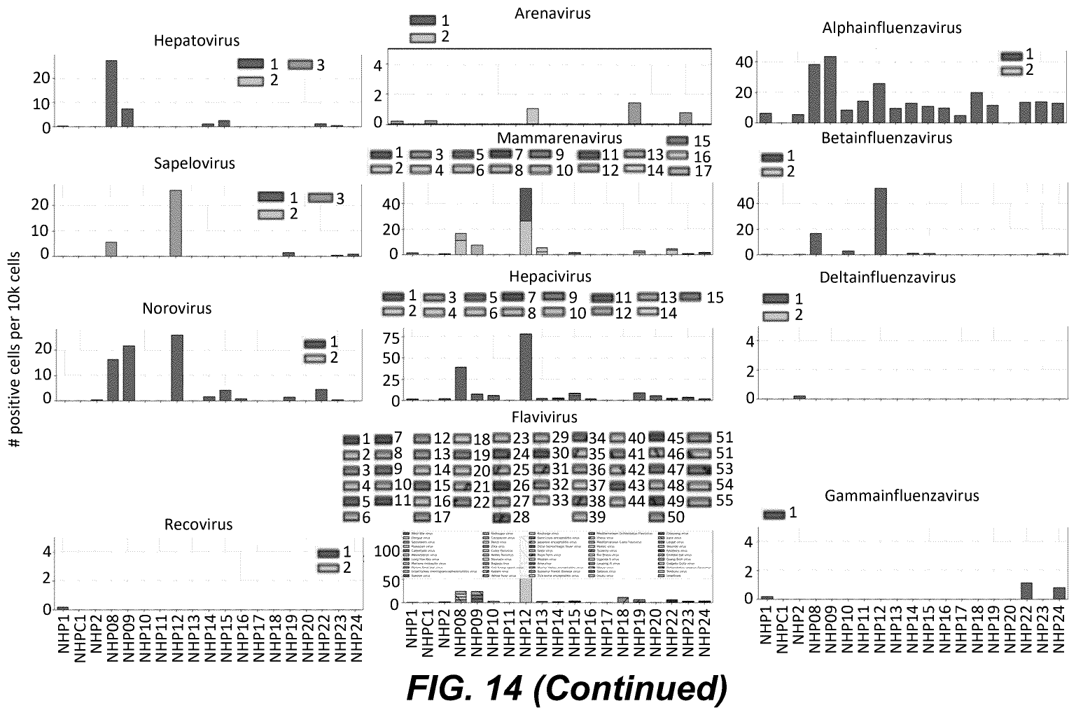

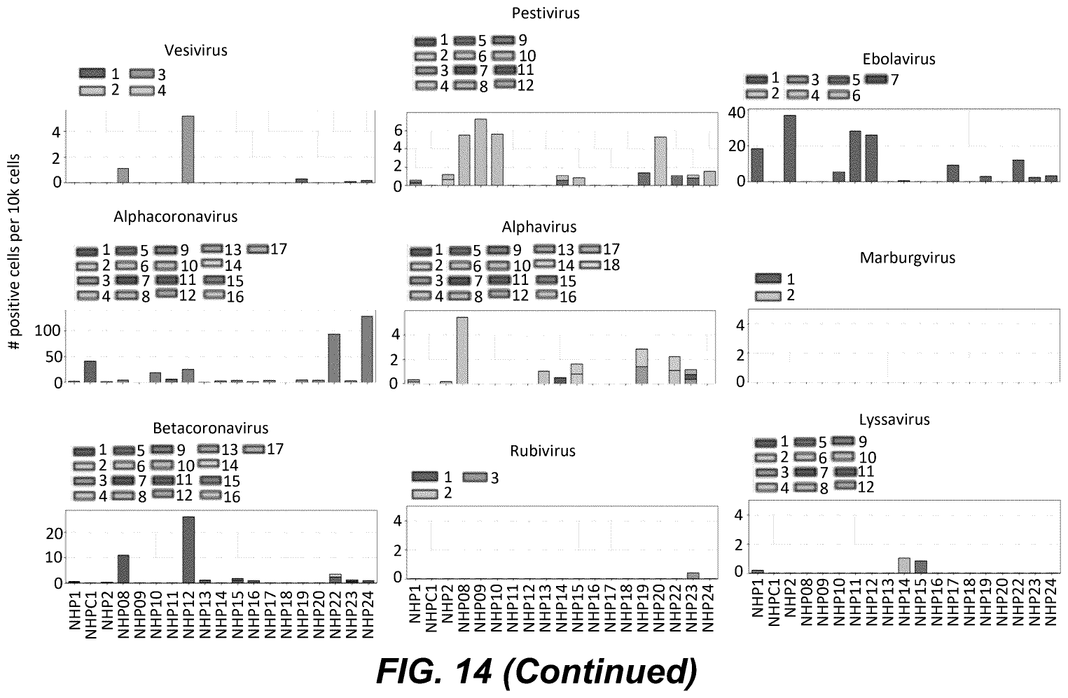

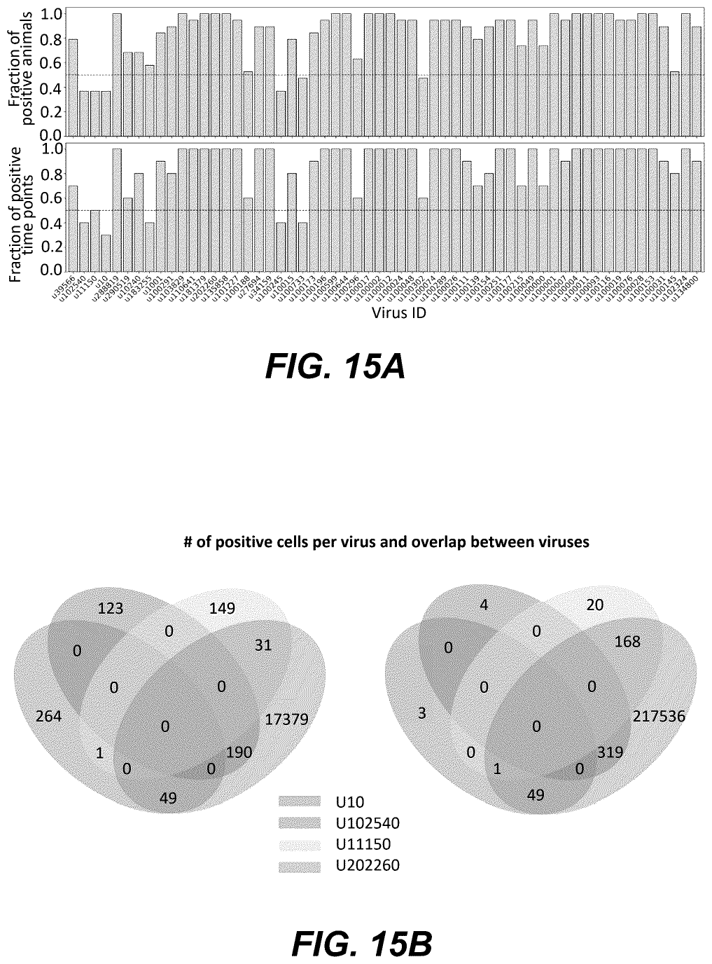

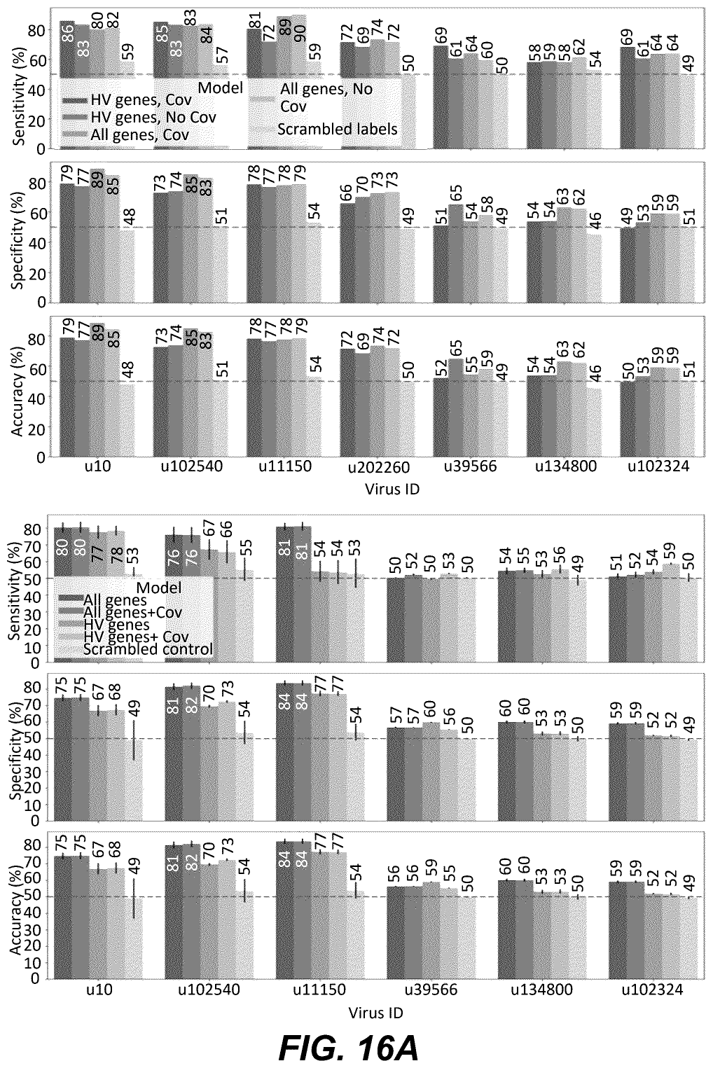

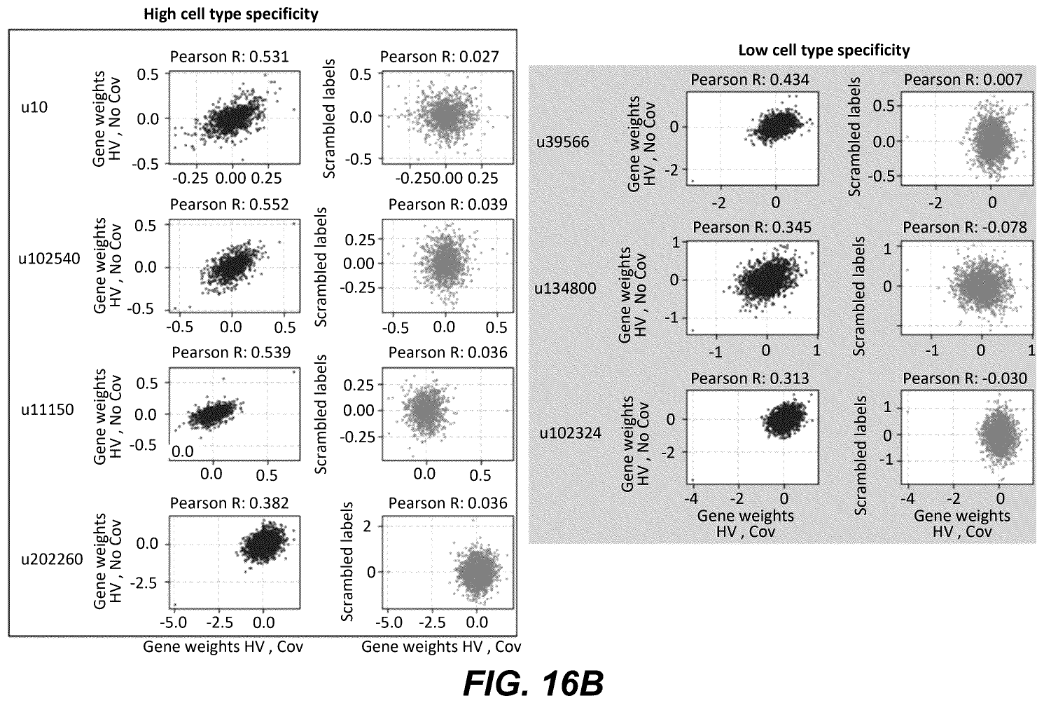

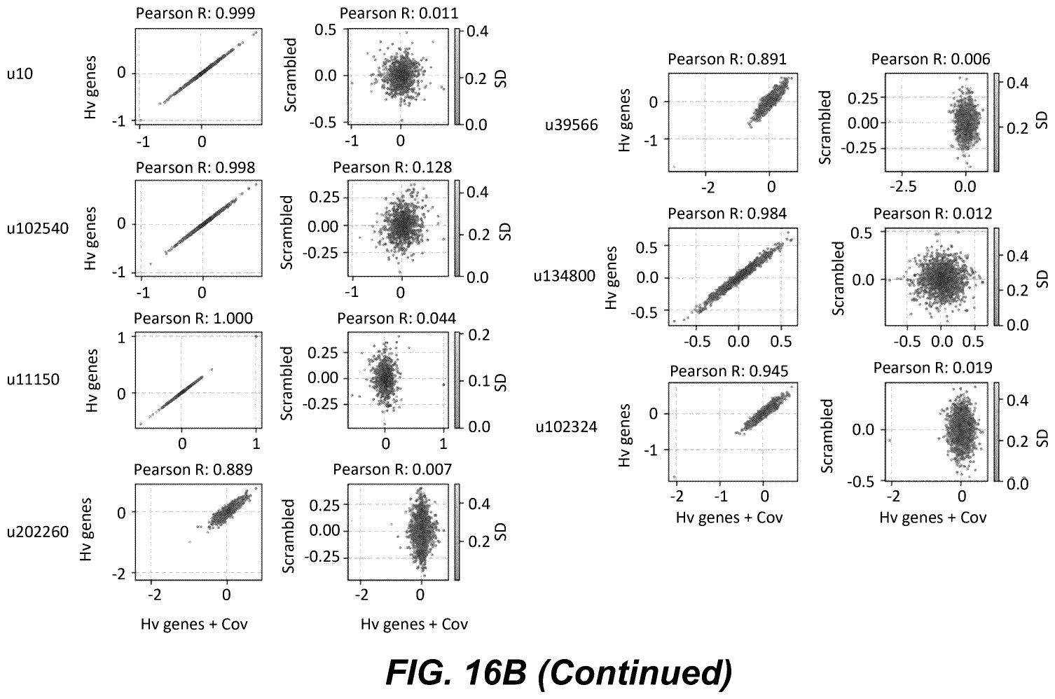

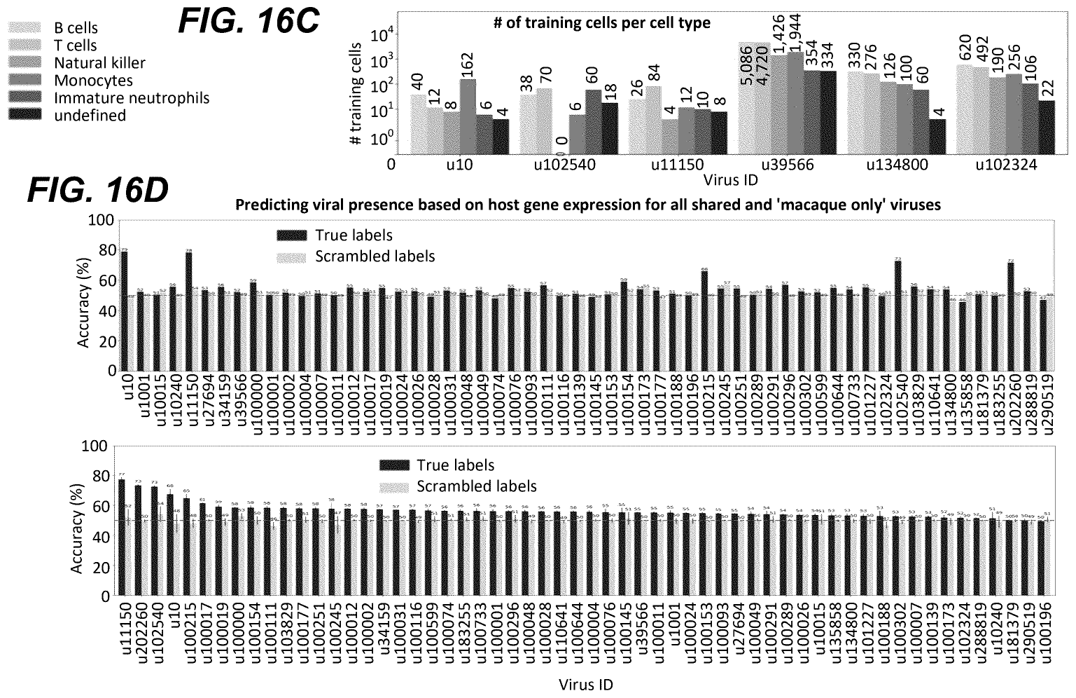

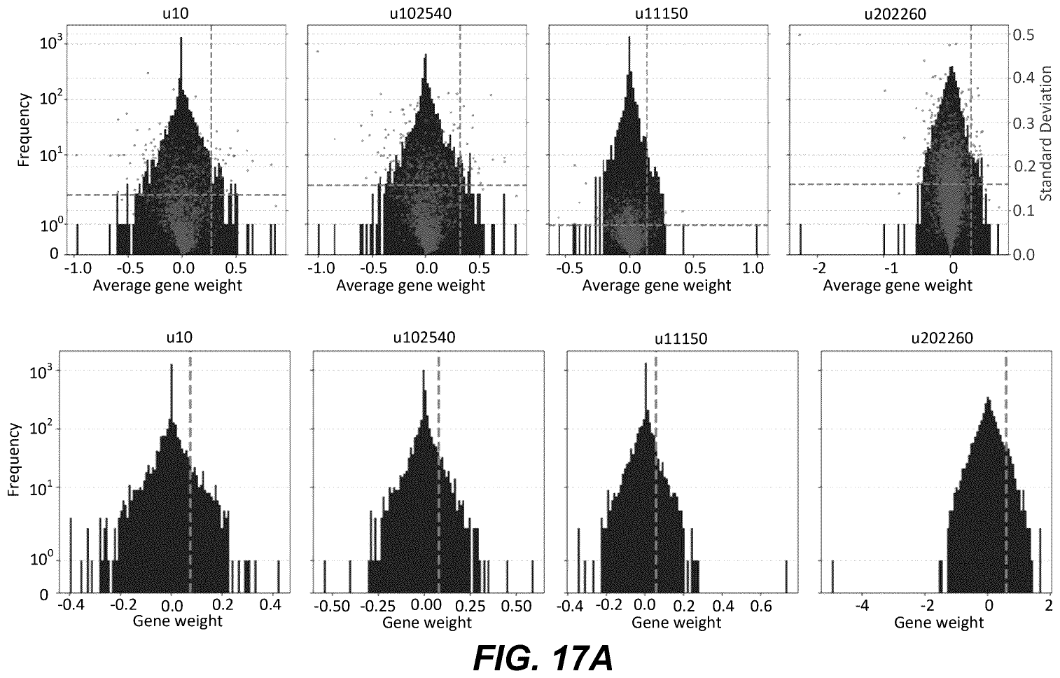

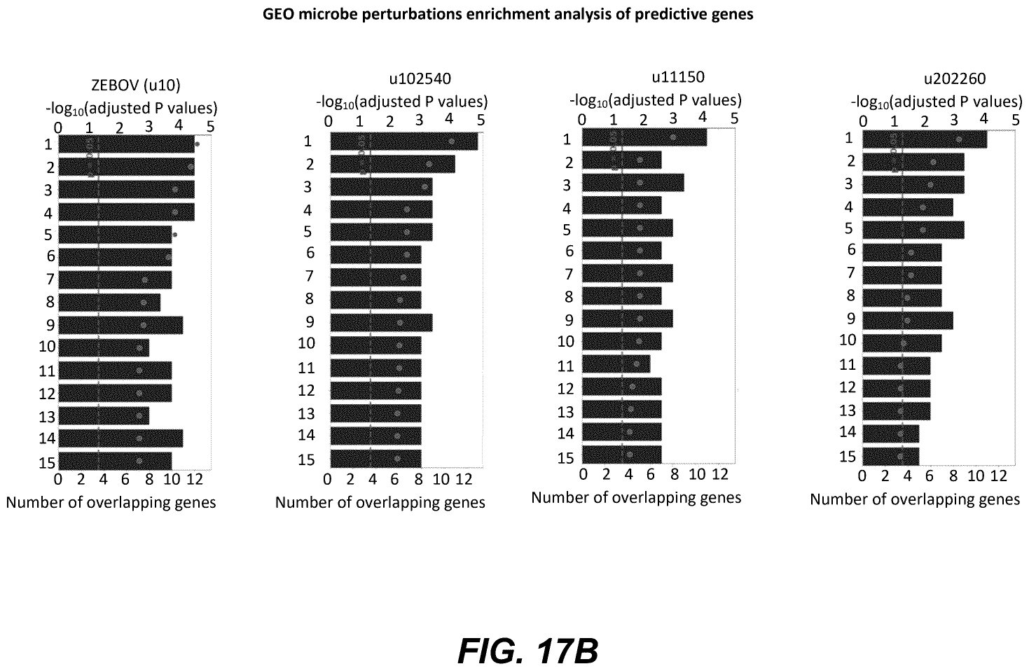

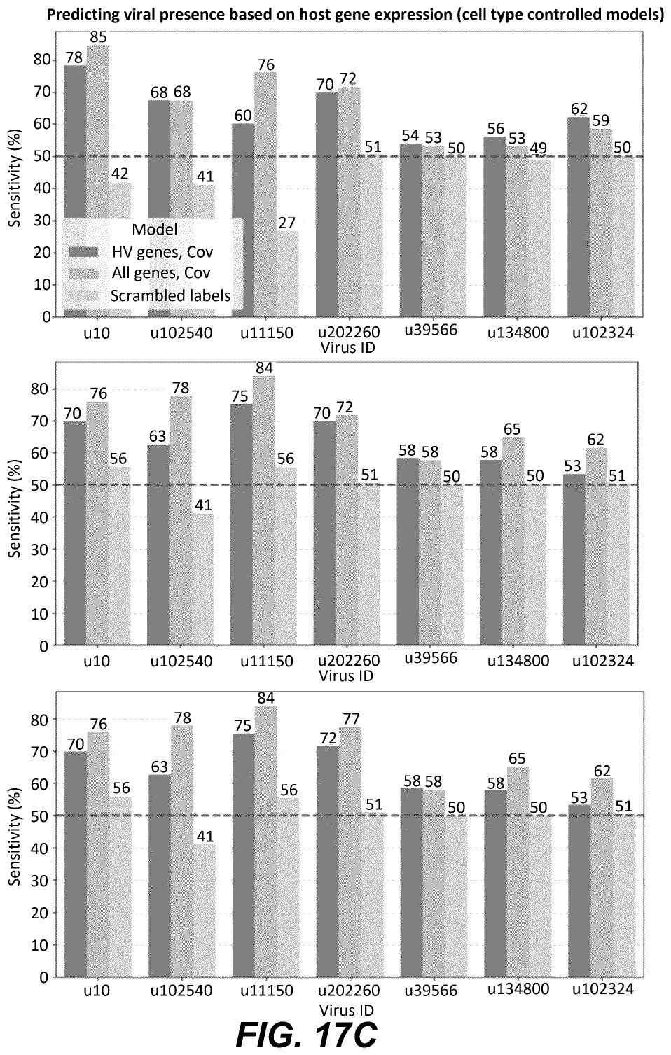

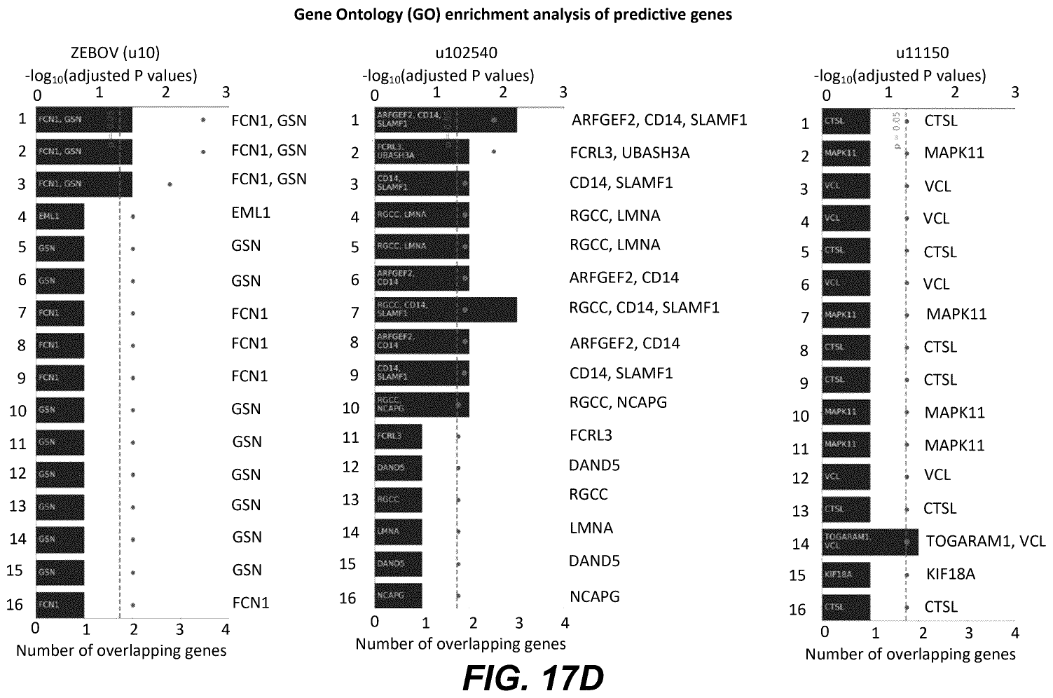

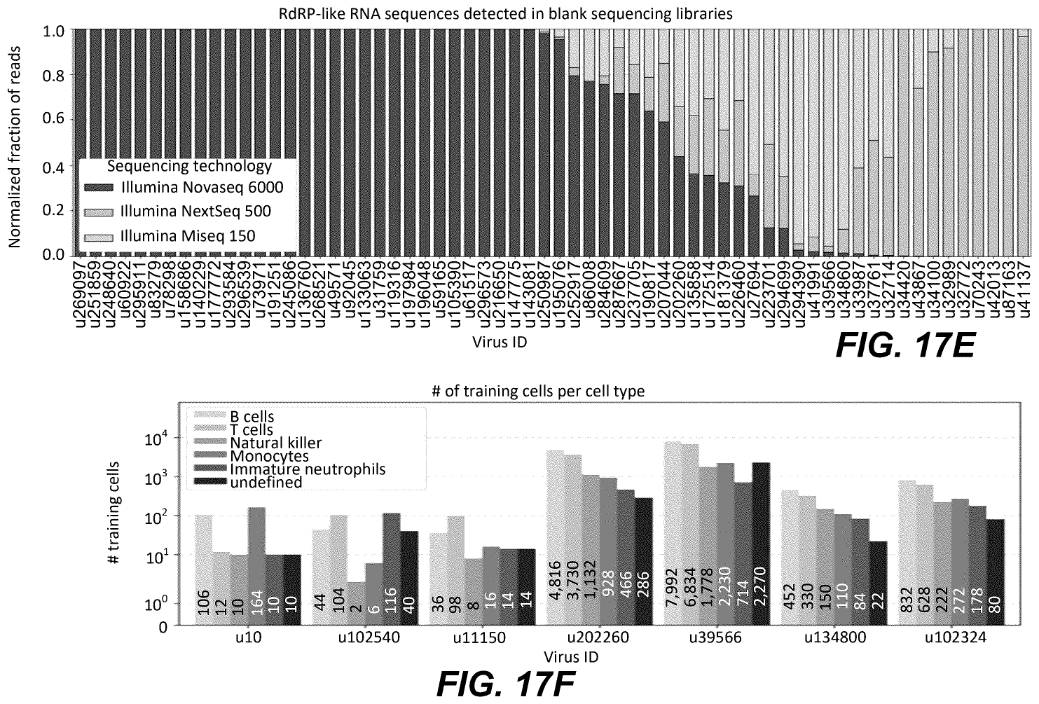

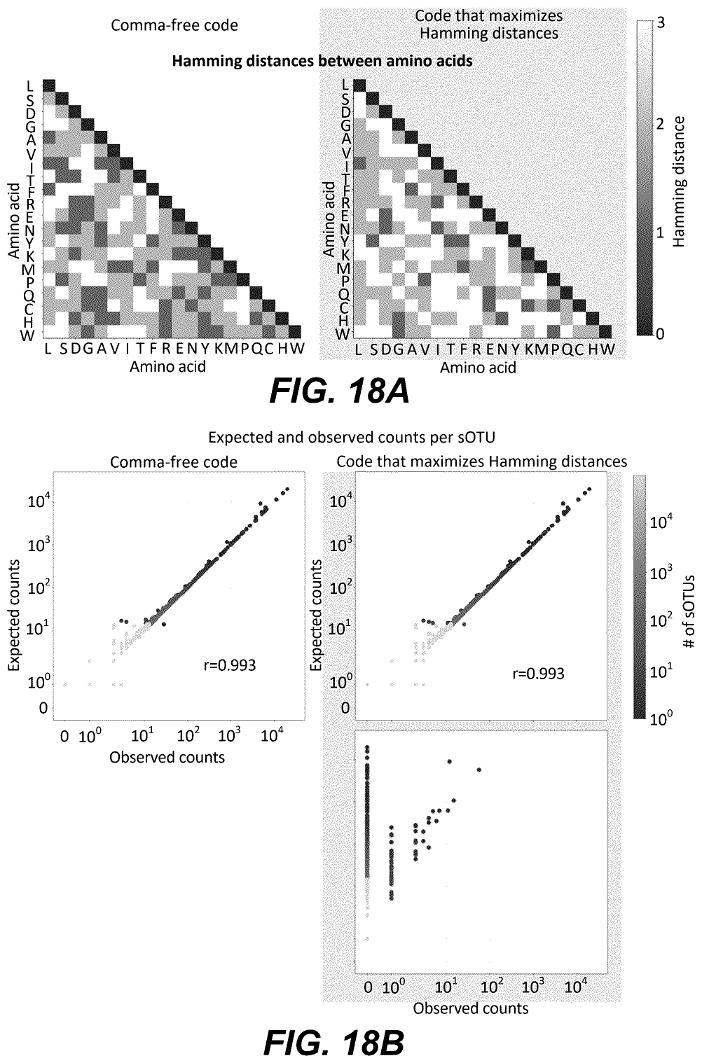

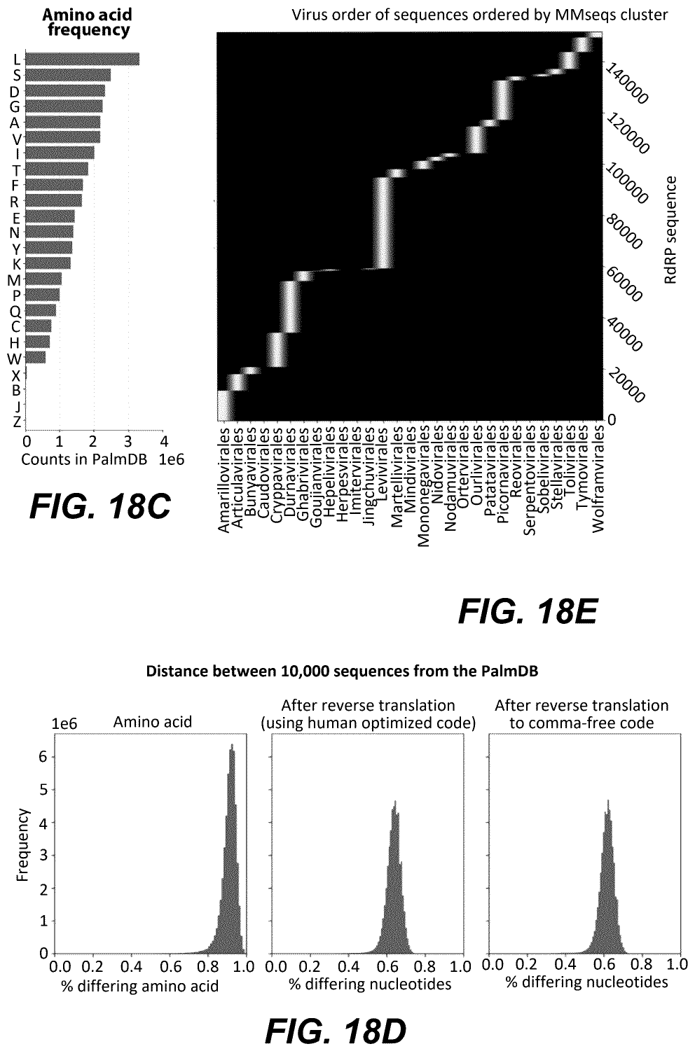

The parallel analysis of viral and host gene counts at single-cell resolution allowed the identification of infected cell types based on host gene expression, revealing that ZEBOV-positive cells consisted predominantly of monocytes (FIG. 7B, third panel from top). These results were consistent with previous literature on ZEBOV tropism and reproduced the ZEBOV abundance trends obtained by alignment to the ZEBOV genome. This indicates that while the total viral counts obtained by kallisto translated search with PalmDB were low due to the detection of only the RdRP, comparative trends were captured accurately. All Ebola virus reads were identified correctly as ZEBOV with no counts detected for other Ebola virus species (FIG. 14).

The analytic workflow disclosed herein identified viruses other than ZEBOV in this dataset. These viruses may be present due to infection of the host, host endogenous viral elements, infection of bacteria residing in the host, infection of food ingested by the host or laboratory contamination. The second from top and bottom panels of FIG. 7B show the total number of distinct sOTUs detected over time and per cell type, which corresponded to distinct virus IDs. A slight increase in the number of distinct sOTUs detected per cell was observed during the later stages of EVD, driven by T cell, B cell, and neutrophil clusters, as evidenced by high fractions of B cells, T cells and neutrophils during later EVD stages (FIG. 7A). Neutrophils showed the highest numbers of distinct viruses per cell (FIG. 7B, bottom panel). Since neutrophils fulfilled their microbicidal function through phagocytosis and pinocytosis, it is possible that the viral RNA was picked up by these cells through ingestion rather than cellular infection. Different approaches to interpret the presence of these virus-like sequences were also explored and described in the following paragraphs.

Among the samples in this dataset, a total of 11,176 viruses were detected with at least one read that aligned to the PalmDB and did not align to the host (FIG. 4C), including many viruses from genera known to infect rhesus macaques (FIG. 14). However, the majority of these viruses were expressed in less than 0.05% of cells, which did not pass the quality control (QC) threshold (FIG. 6B). The QC threshold was defined as being detected in ≥0.05% of cells. All of the viruses with positive cell fractions above the QC threshold in macaque cells that passed quality control can be explored in the interactive Krona plot broken down by animal, time-point, taxonomy, and fraction of cells occupied by each virus at tinyurl.com/23h6k36u.

A subset of samples included a spike-in of Madin-Darby canine kidney (MDCK) cells, resulting in a total of 23,500 MDCK cells after quality control and species separation (FIG. 11B and FIG. 11C). The spike-in was used to further break down the viruses into “macaque only,” “MDCK only,” and “shared” viruses (FIG. 6B). It was expected that shared viruses occurring in both macaque and MDCK cells would include viruses introduced by the contamination of laboratory reagents used during sample preparation and sequencing, cell-free RNA contamination, endogenous retroviruses and widespread latent infections After filtering and categorization of viruses, 4 macaque only, 7 MDCK only, 15 undefined and 54 shared viruses were detected. The 4 macaque only viruses included ZEBOV. This result suggested that the majority of virus-like sequences detected in this dataset were introduced through contamination. Indeed, many virus-like sequences that fell into the shared category could also be detected in ‘blank’ sequencing libraries containing only sterile water and reagent mix, providing evidence for their origin in widespread laboratory reagent contamination (FIG. 6B and FIG. 17E). The sOTUs of the macaque only and shared virus IDs are listed in Table 8. FIG. 6C shows the fraction of reads occupied by each viral order for macaque only, MDCK only, and shared viruses. The number of total cells in each viral order is listed in Table 8 below. In the third column of Table 8, virus IDs also detected in the macaque dataset are indicated with “*.”

### Table 8: Total Cells in Each Viral Order in FIG. 6C and Total Reads for Each Virus ID in FIG. 17E

| Correspond to FIG. 6C | | Correspond to FIG. 17E | |
|---|---|---|---|
| Virus order | Number of cells | Virus ID | Total reads |
| Articulavirales | 9,832 | u269097 | 3,270 |
| Cryppavirales | 2,625 | u251859 | 3,216 |
| Durnavirales | 2,694 | u248640 | 2,821 |
| Ghabrivirales | 433 | u60922 | 2,145 |
| Herpesvirales | 478 | u295911 | 2,144 |
| Levivirales | 11,162 | u83279 | 2,055 |
| Martellivirales | 275 | u78298 | 1,268 |
| Ourlivirales | 2,149 | u158686 | 1,201 |
| Reovirales | 1,855 | u140229 | 986 |
| Sobelivirales | 138 | u177772 | 875 |
| Tolivirales | 605 | u293584 | 869 |
| Picornavirales | 3,330 | u296539 | 761 |
| Wolframvirales | 650 | u73971 | 757 |
| Amarillovirales | 59 | u191251 | 730 |
| Bunyavirales | 553 | u245086 | 713 |
| Undefined | 67,347 | u136760 | 624 |
| Nidovirales | 2,841 | u268521 | 579 |
| Mononegavirales | 621 | u49571 | 573 |
| | | u92045 | 563 |
| | | u133063 | 530 |
| | | u31759 | 524 |
| | | u119316 | 501 |
| | | u197984 | 491 |
| | | u196048 | 479 |
| | | u59165 | 478 |
| | | u105390 | 477 |
| | | u61517 | 445 |
| | | u296573 | 442 |
| | | u216650 | 443 |
| | | u147775 | 415 |
| | | u143081 | 100,518 |
| | | u250987 | 127,623 |
| | | u195076 | 26,107 |
| | | u252917 | 57,752 |
| | | u86008 | 467 |
| | | u284609 | 220,915 |
| | | u287667* | 85,334 |
| | | u237705 | 752,771 |
| | | u190817 | 112,795 |
| | | u207044 | 161,055 |
| | | u202260* | 456,424 |
| | | u135858* | 162,212 |
| | | u172514 | 5,374,210 |
| | | u181379* | 275,183 |
| | | u226460 | 4,301,784 |
| | | u27694* | 3,617 |
| | | u223701 | 359,783 |
| | | u294699 | 2,262 |
| | | u294390 | 9,135 |
| | | u41991* | 844 |
| | | u39566* | 7,283 |
| | | u34860* | 874 |
| | | u33987* | 1,790 |
| | | u37761* | 1,797 |
| | | u32714 | 660 |
| | | u34420 | 1,195 |
| | | u43867 | 948 |
| | | u34100* | 1,960 |
| | | u32989 | 4,609 |
| | | u32772* | 2,828 |
| | | u70243 | 768 |
| | | u42013* | 702 |
| | | u87163 | 558 |
| | | u41137 | 1,526 |

Among viruses shared between macaque and MDCK cells, Levivirales, which was renamed Norzivirales. Articulavirales, which include the family of influenza viruses, and viruses of unknown taxonomy made up the largest fractions. Norzivirales are an order of bacteriophages, the majority of which were discovered in metagenomics studies. They might have been introduced through bacterial contamination during sample preparation and sequencing. The shared viruses also included orders such as Herpesvirales, which are Widespread, sometimes spreading through cross-species infections, and are known to persist in their host as latent infection Virus-like sequences detected in MDCK cells included sOTUs from the order of Bunyavirales, which infect a wide range of hosts, including MDCK cells, as well as virus-like sequences of unknown order. Virus-like sequences found only in macaque cells were of unknown order, in the order Mononegavirales, and in the order Nidovirales. The order Nidovirales is known to infect mammals and includes the family Coronaviridae. ZEBOV is in the order Mononegavirales. Virus-like sequences of known order based on the sOTU for each group were reasonably expected to be present in the respective sample types and the context of the hosts, which supported the biological validity of these viral read classifications.

To visualize the virus profiles of individual animals and over time, the fractions of positive cells for each macaque only and shared virus ID per animal and time point were plotted (FIG. 6D). The relative viral abundances varied, both between individual monkeys and time points. Notably, in some instances where the same animal was measured across several time points, the viral profile of this animal was reproduced in the later time point (FIG. 6D and FIG. 8A). The viral profiles of animal NHP08 4 days before infection and 6 days post-infection with ZEBOV are highlighted in the heatmap (FIG. 6D). Animals NHP1 and NHP2 each had two samples sequenced 20 hours apart, which displayed highly similar viral profiles for each animal over time (FIG. 8A). This suggests that viral profiles sampled and sequenced within a short time window were coherent over time and across samples, which was consistent with expectation. Several viruses, including u102324, were present in all animals and time points with relatively similar abundance (FIG. 6D and FIG. 15A), coherent with their classification as shared sequences likely originating from contamination.

It was also determined which viruses were likely present due to infection of the host based on cell-type specificity and a coinciding host antiviral response. Other than ZEBOV, the viral tropism of three viruses that displayed relatively high sample-specificity, u102540, u11150, and u202260, and three viruses that were abundant across all samples, u39566, u134800, and u102324, similar to the shared viruses u134800 and u102324 (FIG. 15A), were visualized. Three of them (e.g., u102540, u11150 and u39566) were macaque only viruses. The sOTU of u102540 indicated that it was an Alphacoronavirus sp., which are known to infect rhesus macaques. u102324 was predicted to belong to the family Iflaviridae (Table 3), which is a family of viruses that infect insects, and the viral reads from this virus ID were likely not the result of an ongoing viral infection. The remaining 4 virus IDs (e.g., u11150, u202260, u39566 and u134800) were of unknown taxonomy across all taxonomic ranks. There was little cellular overlap between these viruses, as well as with the known infection with ZEBOV (u10), in the virus count matrix (FIG. 15B).

Several viruses exhibited cell-type specificity suggestive of infection. Of the macaque only virus IDs excluding ZEBOV, u102540, u11150, and u202260 showed high cell type specificity, while u39566, u134800, and u102324 were expressed more evenly across all cell types (FIG. 7C). While u39566 was categorized as “macaque only” above, it is likely a contaminating sequence given its presence in the blank sequencing libraries (FIG. 17E). The total reads of virus ID shown in FIG. 17E is listed in Table 8 above. The lack of cell-type specificity coincided with u39566 sequences originating from reagent contamination and illustrated the importance of combining several different approaches, as described here, when interpreting the presence of virus-like sequences. u102540 (Alphacoronavirus sp.) exhibited high fractions of positive cells in neutrophils, while u11150 and u202260 also displayed lower expression in monocytes, B cells and T cells. Neutrophils play an important role in the innate immune response and promote virus clearance through phagocytosis. During phagocytosis, neutrophils engulf virions and apoptotic bodies. It is possible that the cell type specificity towards neutrophils observed here was due to neutrophils engulfing viral RNA during phagocytosis rather than viral tropism. As expected, the shared viruses u134800 and u102324 did not display cell type specificity (FIG. 7C).

The simultaneous analysis of the host and virus count matrices supported that several viruses identified were likely infecting the host and revealed virus-induced host gene expression. Thus, viral presence in individual cells was predicted based on the host gene expression. Since the workflow disclosed herein maintained single-cell resolution, viral presence and host gene expression can be analyzed at single-cell resolution in parallel, and whether the presence of a virus affected host gene expression was also investigate. Logistic regression models were trained for u102540, u11150, u20226, u39566, u134800 and u102324 to predict viral presence or absence in individual cells based on the cell's host gene expression. The models were either trained on all or only highly variable macaque genes and with or without the covariates donor animal and time point. After being trained using a random selection of virus-positive and an equal number of virus-negative cells, the model predictions on held-out test cells were tested (FIG. 8B and FIG. 16A). Given the cell type specificity of several of the virus-like sequences, virus-negative training cells were selected to be of the same cell types as virus-positive cells to ensure prediction of viral presence rather than cell type. While this slightly decreased prediction accuracy, viruses displaying cell type specificity could still be predicted with greater accuracy than those without (FIG. 17E).

The presence or absence of viruses that displayed cell type- and sample-specificity (e.g., u10 (ZEBOV), u02540. u11150 and u202260) could be predicted at >70% accuracy across models (FIG. 8B and FIG. 8C). The sensitivity and specificity are shown in FIG. 16A. By contrast, the presence of viruses that did not display cell type-specificity (u39566, u134800 and u102324) could not be predicted better than random chance (50%) (FIG. 8B and FIG. 8C). As a negative control, the binary virus count matrix was scrambled for model training, effectively randomizing the presence or absence of a virus in each cell. As expected, the prediction accuracies dropped to those expected at random (50%) (FIG. 8B). This learnable relationship between viral presence and host gene expression provided further evidence that reads from u10, the known infection with ZEBOV, as well as novel virus-like sequences (e.g., u102540, u11150 and u202260) originated from an ongoing viral infection and/or viral clearance, which perturbed host gene expression at the single-cell level.

One virus-like sequence, which displayed prediction accuracies >70%, was u202260 (FIG. 16D and Table 9 below). This was surprising, as u202260 was categorized as shared between macaque and MDCK cells and was also present in the blank sequencing libraries (FIG. 6B and FIG. 17E), indicating that it likely originated from laboratory reagent contamination. However, although its prediction accuracies were relatively high, the gene weight correlation between different models was low for u202260 (FIG. 16B) and the standard deviation of gene weights within the same model generated with different random seeds was comparatively high (FIG. 17A), indicating that genes were weighted differently between models and seeds for u202260. This suggests that a shared feature across genes, such as cell health or sequencing depth, was learned rather than the expression of specific genes.

TABLE 9
ACCURACY % OF VIRAL PRESENCE
PREDICTION IN FIG. 16D

| Top panel of FIG. 16D | | | Bottom panel of FIG. 16D | | |
|---|---|---|---|---|---|
| | True | Scrambled | | True | Scrambled |
| Virus ID | labels | labels | Virus ID | labels | labels |
| u10 | 79 | 48 | u11150 | 77 | 52 |
| u1001 | 52 | 49 | u202260 | 73 | 50 |
| u10015 | 51 | 52 | u102540 | 73 | 54 |
| u10240 | 56 | 49 | u10 | 68 | 48 |
| u11150 | 78 | 54 | u100215 | 65 | 48 |
| u27694 | 53 | 50 | u100017 | 61 | 50 |
| u34159 | 56 | 51 | u100019 | 59 | 49 |
| u39566 | 52 | 49 | u100000 | 58 | 53 |
| u100000 | 59 | 51 | u100154 | 58 | 50 |
| u100001 | 50 | 50 | u100111 | 58 | 46 |
| u100002 | 52 | 49 | u103829 | 58 | 50 |
| u100004 | 50 | 51 | u100177 | 58 | 51 |
| u100007 | 51 | 49 | u100251 | 58 | 50 |
| u100011 | 50 | 49 | u100245 | 58 | 47 |
| u100012 | 55 | 50 | u100012 | 58 | 50 |
| u100017 | 52 | 51 | u100002 | 58 | 50 |
| u100019 | 55 | 47 | u34159 | 57 | 50 |
| u100024 | 53 | 51 | u100031 | 57 | 50 |
| u100026 | 53 | 50 | u100116 | 57 | 49 |
| u100028 | 49 | 51 | u100599 | 57 | 51 |
| u100031 | 53 | 50 | u100074 | 56 | 51 |
| u100048 | 52 | 48 | u183255 | 56 | 51 |
| u100049 | 53 | 50 | u100733 | 56 | 51 |
| u100074 | 48 | 49 | u100001 | 56 | 50 |
| u100076 | 55 | 52 | u100296 | 56 | 51 |
| u100093 | 52 | 50 | u100048 | 56 | 49 |
| u100111 | 57 | 52 | u100028 | 56 | 50 |
| u100116 | 50 | 49 | u110641 | 56 | 49 |
| u100139 | 51 | 48 | u100644 | 56 | 50 |
| u100145 | 49 | 48 | u100004 | 56 | 50 |
| u100153 | 51 | 50 | u100076 | 55 | 50 |
| u100154 | 59 | 52 | u100145 | 55 | 51 |
| u100173 | 54 | 55 | u39566 | 55 | 50 |
| u100177 | 53 | 47 | u100011 | 55 | 50 |
| u100188 | 51 | 49 | u1001 | 55 | 50 |
| u100196 | 50 | 49 | u100024 | 55 | 50 |
| u100215 | 66 | 49 | u100153 | 55 | 50 |
| u100245 | 55 | 57 | u100093 | 55 | 50 |
| u100251 | 55 | 48 | u27694 | 55 | 50 |
| u100289 | 55 | 51 | u100049 | 54 | 50 |
| u100291 | 54 | 50 | u100291 | 54 | 51 |
| u100296 | 57 | 48 | u100289 | 54 | 50 |
| u100302 | 53 | 49 | u100026 | 54 | 50 |
| u100599 | 52 | 49 | u10015 | 54 | 51 |
| u100644 | 55 | 49 | u135858 | 53 | 50 |
| u100733 | 54 | 49 | u134800 | 53 | 50 |
| u101227 | 55 | 52 | u101227 | 53 | 50 |
| u102324 | 50 | 51 | u100188 | 53 | 47 |
| u102540 | 73 | 51 | u100302 | 53 | 49 |
| u103829 | 56 | 52 | u100007 | 53 | 50 |
| u110641 | 54 | 52 | u100139 | 53 | 50 |
| u134800 | 54 | 46 | u100173 | 52 | 49 |
| u135858 | 46 | 50 | u102324 | 52 | 50 |
| u181379 | 51 | 51 | u288819 | 52 | 50 |
| u183255 | 50 | 49 | u10240 | 51 | 49 |
| u202260 | 72 | 50 | u181379 | 50 | 50 |
| u288819 | 53 | 50 | u290519 | 50 | 49 |
| u290519 | 47 | 49 | u100196 | 50 | 51 |

To explore virus-induced host gene expression, macaque genes with the largest predictive power and smallest variation (across models initialized with different random seeds) were identified for the regression models trained on highly variable genes with the donor animal and time point as covariates (FIG. 17A). Approximately one third of the macaque Ensembl IDs did not have annotated gene names, which is a common problem for genomes from non-model organisms. Gget was used to translate annotated Ensembl IDs to gene symbols and to perform an enrichment analysis on the returned gene symbols using Enrichr against the 2023 Gene Ontology (GO) Biological Processes database. The highly weighted genes for u10 (ZEBOV) returned significant enrichment results for several virus-associated GO terms including “Negative Regulation of Viral Entry into Host Cell (GO: 0046597),” “Negative Regulation of Viral Life Cycle (GO: 1903901),” and “Regulation of Viral Entry into Host Cell (GO: 0046596),” validating the approach disclosed herein for the identification of genes associated with a virus-related host gene response. Similarly, the enrichment analysis of highly weighted genes for the novel virus ID u11150 mapped to “Receptor-Mediated Endocytosis of Virus by Host Cell (GO: 0019065).” For virus ID u102540, several highly ranked GO terms were indicative of an inflammatory response, such as “Positive Regulation of Type II Interferon Production (GO: 0032729)” and “Positive Regulation of Cytokine Production (GO: 0001819).” Several predictive genes were associated with the positive regulation of cytokine production and modulation of inflammation (e.g., FCN1 for u 10, MAPK11 for u11150 and CD14 for u102540). Overall, these results provide further evidence that the novel virus-like sequences (e.g., u102540 and u11150) originated from an ongoing viral infection or clearance resulting in a host gene response.

In another experiment, the top 200 macaque genes with the largest predictive power in the regression model trained on highly variable genes with covariates donor animal and time point were analyzed. Approximately half of the macaque Ensembl IDs did not have annotated gene names, which is a common problem for genomes from non-model organisms. Gget was used to translate annotated Ensembl IDs to gene symbols and to perform enrichment analysis on the returned gene symbols using Enrichr against the GEO microbe perturbations database. The highly weighted genes for all viruses that were predicted with high accuracy returned significant enrichment results for microbe perturbations, including many viral infections (FIG. 17B). Moreover, the human KEGG database was used to identify predictive genes involved in cellular processes associated with viral infections. Some of the predictive genes were associated with pathways involved in the identification of viral invasion by the innate immune response, such as cytokine-cytokine receptor interactions (e.g., CCL24, IL1RL1, IFNG, TGFB3, TNFSF10, INHBB and CCL17 for u10; TNFSF10 and PF4 for u102540; ACVRL1, CXCL9, CCL4L1, TNFRSF10A and CCL18 for u11150; and IL21, IL10, CXCR1 and IL1R2 for u202260), extracellular matrix (ECM) receptor interactions (e.g., RELN, VWF, ITGA2 and COL6A5 for u10; ITGA2 and HMMR for u102540; RELN for u11150; and ITGA3, ITGA2B, TNR, ITGA7, CD36 and THBS1 for u202260), and the expression of NOD-like receptors (e.g., MAPK11 and OAS2 for u102540; MAPK11, DHX33 and TP53BP1 for u11150; and TRPM2, JUN and OAS3 for u202260). Several predictive genes were involved in signaling pathways for the activation and modulation of inflammation, such as IL-17, a key cytokine in neutrophil mobilization (e.g., IFNG, CCL17 and S100A8 for u10; MAPK11, LCN2 and S100A9 for u102540; MAPK11 for u11150; and JUN for u202260), nuclear factor kappa-light-chain-enhancer of activated B cells (NF-κB) (e.g., GADD45G for u10; BLNK for u102540; and CCL4L1, ERC1 and GADD45G for u11150), Peroxisome Proliferator-Activated Receptor gamma (PPAR) (e.g., FABP4 and CD36 for u202260), and forkhead box O (FoxO) (e.g., TGFB3, TNFSF10, IRS2 and GADD45G for u10; MAPK11, TNFSF10 and IGF1R for u102540; MAPK11, IRS1, IRS2, GADD45G and IGF1R for u11150; and IL10 for u202260). Other predictive genes were involved in cellular antiviral responses, such as the induction of inflammatory cell death (apoptosis and necroptosis) (e.g., TNFSF10, PRF1, GADD45G, H2AC6 and IFNG for u10; CTSL, LMNA, TNFSF10, PRF1 and H2AC6 for u102540; CTSL, LMNA, TNFRSF10A, GADD45G and H2AC6 for u11150; and JUN, CAMK2B and FTL for u202260), or membrane-associated responses, such as neutrophil extracellular trap formation (e.g., SELP, H2AC6 and VWF for u10; MAPK11 and H2AC6 for u102540; MAPK11, H2AC6 and H2BC5 for u11150; and ITGA2B for u202260), and Fc gamma R-mediated phagocytosis (e.g., PLPP1 for u10; MYO10 and PLPP1 for u102540; and PLPP1 for u11150).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for detecting microbes in a sample, comprising:
converting a plurality of reference sequences to a plurality of comma-free reference codes;
converting a plurality of sample sequences to a plurality of comma-free sample codes; and
aligning the plurality of comma-free reference codes to the plurality of comma-free sample codes to generate a microbe profile of the sample, thereby detecting the presence of one or more microbes in the sample.

2. The method of claim 1, further comprising removing sample sequences of the plurality of sample sequences originated from host.

3.-4. (canceled)

5. The method of claim 1, further comprising:
converting host sequences to a plurality of comma-free host codes;
aligning the plurality of comma-free sample codes to the comma-free host codes, and
removing comma-free sample codes of the plurality of comma-free sample codes that comprise a portion aligned to a host specific sequence.

6.-9. (canceled)

10. The method of claim 1, further comprising removing comma-free sample codes of the plurality of comma-free sample codes that lack a reference specific sequence, wherein the reference specific sequence aligns to the plurality of comma-free reference codes but not the comma-free reference codes comma-free host codes.

11.-12. (canceled)

13. The method of claim 1, further comprising:
comparing the alignment of the plurality of comma-free sample codes associated with a sample sequence of the plurality of sample sequences to the plurality of comma-free reference codes; and
selecting the comma-free sample codes of the plurality of comma-free sample codes having the highest similarity to the comma-free reference codes compared to other comma-free sample codes associated with the same sample sequence for subsequence analysis.

14.-17. (canceled)

18. The method of claim 1, wherein the plurality of reference sequences comprise RNA-dependent RNA polymerase (RdRp)-containing amino acid sequences and/or antimicrobial amino acid sequences.

19. The method of claim 1, wherein the length of each of the plurality of comma-free reference codes is 10-3000 nucleotides.

20.-24. (canceled)

25. The method of claim 1, wherein each of the plurality of comma-free reference sequences comprises taxonomy source information of its corresponding reference sequence.

26.-30. (canceled)

31. The method of claim 1, wherein the plurality of sample sequences comprise at least one mutation and wherein the mutation rate of the plurality of sample sequences is no greater than 20%.

32.-35. (canceled)

36. The method of claim 1, wherein the microbe profile comprises taxonomy, number and tropism of microbes of the microbes.

37.-39. (canceled)

40. The method of claim 1, further comprising determining:
(1) profile of cells in the sample, wherein the profile of the cells comprises expression level of genes known to be associated with microbe infection, type of cells infected with the microbe and abundance of each type of cells infected with the microbe;
(2) the percentage of cells infected with the microbe; or
(3) the stage of microbe infection.

41.-42. (canceled)

43. The method of claim 40, wherein the genes known to be associated with microbe infection are selected from the group consisting of MS4A1, CD19, CD79B, MZB1, IRF8, CD1C, IL7R, CD8A, CD3D, CD3G, CD3E, CD4, GZMB, KLRB1, NCR1, FCGR3, HLA-DRB5, HLA-DRA, CD68, ITGAX, CD14, ITGAM, CFD, CD163, SOD2, LCN2, CD4177, CD45, IL-1β, CCL2, CCL3, CCL4 and Ki67.

44.-46. (canceled)

47. The method of claim 1, wherein the method detects more microbes compared to a method aligning the plurality of sample sequences to NCBI reference sequences and microbes without a sequence included in the NCBI database or in the plurality of reference sequences.

48.-49. (canceled)

50. The method of claim 1, wherein the method generates microbe profile with at least 90% accuracy.

51. A method for predicting or detecting microbes in a sample, comprising:
providing a model with a training dataset to determine a weight of each gene in the training data, wherein the model is a logistic regression modal, and wherein the training dataset comprises sequencing data of one or more cells;
determining one or more signature genes, wherein the signature genes have weights no less than a threshold;
providing a trained model with a testing dataset, wherein the trained model is parameterized with the weight of the signature genes and wherein the testing dataset comprises sequencing data of one or more cells in the sample; and
determining a probability of presence of the microbes using the trained model, thereby determining the presence or absence of the microbes in the sample.

52. (canceled)

53. The method of claim 51, wherein the microbe is a virus from the realm of Riboviria.

54.-57. (canceled)

58. The method of claim 51, wherein the training dataset comprises cell type and infection status of each cell of one or more cells infected or suspected to be infected with the microbes and highly variable genes in the one or more cells.

59.-64. (canceled)

65. The method of claim 51, wherein the threshold is 0.01.

66. The method of claim 51, wherein the signature genes are genes encoding:
proteins regulating cytokine production,
proteins regulating viral entry into host cell,
proteins regulating viral life cycle, and/or
receptors mediating endocytosis.

67. The method of claim 51, wherein the signature genes are genes encoding proteins selected from the group consisting of FCN1, GSN, EML1, ARFGEF2, CD14, SLAMFI, FCRL3, UBASH3A, RGCC, LMNA, NCAPG, FCRL3, DAND5, CTSL, MAPK11, VCL, TOGARAM1 and KIF18A.

68.-71. (canceled)
